(19) United States
(12) Patent Application Publication
(10) Pub. No.: US 2019/0117534 A1
(43) Pub. Date: Apr. 25, 2019

(54) Grip Enhancing Compositions, and Methods and Uses Thereof

(71) Applicant: Collidion, Inc., Petaluma, CA (US)

(72) Inventor: Nicholas Kamron Alimi, Petaluma, CA (US)

(73) Assignee: Collidion, Inc., Petaluma, CA (US)

(21) Appl. No.: 16/169,952

(22) Filed: Oct. 24, 2018

Related U.S. Application Data
(60) Provisional application No. 62/576,326, filed on Oct. 24, 2017.

(51) Int. Cl. A61K 8/25; A61K 8/73; A61Q 19/00

(52) U.S. Cl. CPC A61K 8/25; A61K 8/731; A61K 2800/87; A61K 2800/74; A61Q 19/00

(57) ABSTRACT

The present specification discloses a composition comprising, consisting essentially of, or consisting of one or more grip enhancers, one or more hydrophilic clays, and/or one or more polysaccharides, kits comprising, consisting essentially of, or consisting of a disclosed composition, methods and uses to improve the grip capacity of an individual by applying a disclosed composition, and methods and uses to improve the grip capacity of an object by applying a disclosed composition.

This application claims the benefit of priority and the filing date pursuant to 35 U.S.C. § 119(e) to U.S. Provisional Patent Application 62/576,326, filed Oct. 24, 2017, the entire content of which is hereby incorporated by reference in its entirety.

Many types of exercise, athletic endeavors, or other physical exertion require that a participant achieve and/or maintain a secure grip of a piece of equipment, apparatus, implement, or other object to competently and safely execute an activity being performed. However, grasping a gripping portion of such objects is generally made difficult when it becomes wet, such as, e.g., due to perspiration or environmental moisture. Such wet conditions can result in slippage of the participant's grip, which can negatively impact performance of the participant, can be particularly dangerous to the participant, and can pose a severe threat to harm to others near the activity. Therefore, improving the grip of a participant under such wet conditions is critical to any exercise, athletic endeavors, or other physical exertion where a secure grip of a piece of equipment, apparatus, implement, or other object is required for the participant to perform an activity in a proficient and safe manner.

To improve grip of a participant, various materials have been developed as grip enhancers which can be applied to the skin of the participant and/or the griping portion of an object, including chalk, talc, resin, or rosin. Chalk is a common grip enhancer used by many participants where a secure grip is desired. When rubbed on the skin, this compound forms a thin layer that acts as an antiperspirant by absorbing sweat, and adding texture to skin and surfaces, leading to the maintenance, restoration, and/or improvement in frictional forces, to improve grip. Traditionally, chalk was formulated as a compressed block or a powder. However, besides creating a mess caused by spills, overuse or the like, use of such formulations are undesired as it introduces chalk dust into the air which can pose health concerns for both the participant and one or more bystanders. For example, chalk dust can land on the skin of an individual to evoke an allergic reaction; get into the eyes of an individual to cause irritation, redness and tearing; or be inhaled by an individual where it can provoke respiratory distress including asthma and infections. There are also environmental concerns with the use of such chalk formulations in outdoor activities like rock climbing since the chalk can corrode the rock formations. The use of talc as a grip enhancer results in the same problems associated with chalk since this is also formulated as a block or powder. The concern has risen to such a state that many indoor environments and outdoor recreation sites have either restricted or banned the use of chalk/talc blocks and powdered chalk/talc.

To address these harmful effects or chalk and talc, liquid grip enhancer compositions have been developed. These compositions typically include powdered chalk or talc suspended in an alcohol such as ethanol or isopropanol. When applied, the liquid composition is spread over the skin surface, where after the alcohol evaporates, the chalk or talc remains on the surface to which the composition was applied. Although such liquid compositions appear to minimize the generation of dust upon initial application, chalk or talc dust is still introduced into the air since once the alcohol evaporates, the chalk or talc applied to the skin surface essentially reverts into a powdered form that can be dispersed into the air by movement or friction.

Another problem associated with alcohol-based liquid chalk compositions is that the alcohol servers as a desiccant that dries out the skin causing cracking and bleeding. Such adverse side effects not only diminish the participant's grip due to the pain associated with these open wounds, but can also increase the participant's susceptibility to subsequent infection.

Thus, there is still a need to develop a grip enhancing composition that addresses the health concerns raised using current compositions containing a grip enhancer.

### SUMMARY

Aspects of the present specification disclose a grip enhancing composition comprising one or more grip enhancers and one or more hydrophilic clays. The disclosed composition may further comprise and one or more carriers and optionally one or more silicones, one or more polysaccharides, one or more fragrances, one or more antimicrobials, one or more carriers, one or more vitamins, free available chlorine and/or hypochlorous acid. The disclosed composition may also not include an alcohol.

Other aspects of the present specification disclose a grip enhancing composition comprising one or more grip enhancers and one or more polysaccharides. The disclosed composition may further comprise and one or more carriers and optionally one or more silicones, one or more hydrophilic clays, one or more fragrances, one or more antimicrobials, one or more carriers, one or more vitamins, free available chlorine and/or hypochlorous acid. The disclosed composition may also not include an alcohol.

Other aspects of the present specification disclose a method to improve the grip capacity of an individual. The disclosed method comprising topically applying a composition disclosed herein to a skin region of the individual, wherein application of the composition to the skin region of the individual restores, maintains and/or improves the grip capacity of the individual allowing the individual to more securely grip an object. Other aspects of the present specification disclose a grip enhancing composition as disclosed herein for use to improve the grip capacity of an individual, use of a grip enhancing composition as disclosed herein to improve the grip capacity of an individual, and use of a grip enhancing composition as disclosed herein in the manufacture of a medicament to improve the grip capacity of an individual.

Other aspects of the present specification disclose a method to protecting a skin region of an individual. The disclosed method comprising topically applying a composition disclosed herein to a skin region of the individual, wherein application of the composition to the skin region of the individual reduces, ameliorates, eliminates or prevents an irritation, a pain or other symptom that causes discomfort or displeasure, thereby providing protection to the skin region. Other aspects of the present specification disclose a grip enhancing composition as disclosed herein for use to protect a skin region of an individual, use of a grip enhancing composition as disclosed herein to protect a skin region of an individual, and use of a grip enhancing composition as disclosed herein in the manufacture of a medicament to protect a skin region of an individual.

Other aspects of the present specification disclose a method to improve the grip capacity of a surface of an object. The disclosed method comprising applying a composition disclosed herein to the surface of the object, wherein application of the composition to the surface of the object restores, maintains and/or improves the grip capacity of an individual gripping the object. Other aspects of the present specification disclose a grip enhancing composition as disclosed herein for use to improve the grip capacity of a surface of an object, use of a grip enhancing composition as disclosed herein to improve the grip capacity of a surface of an object, and use of a grip enhancing composition as disclosed herein in the manufacture of a medicament to improve the grip capacity of a surface of an object.

### DETAILED DESCRIPTION

A grip enhancing composition disclosed herein may comprise, in part, a grip enhancer. A grip enhancer is a compound that improves the grip capacity properties of an individual or a surface, or the connection between an individual and a surface by improving, restoring, or maintaining dryness, texture, and/or friction. Non-limiting examples of a grip enhancer include, without limitation, a chalk, a talc, a resin and a rosin. A chalk is any soft sedimentary rock or soft carbonate mineral, such as, e.g., limestone and includes without limitation magnesium carbonate (MgCO₃) and calcium carbonate (CaCO₃), see, e.g., U.S. Pat. Nos. 1,163,475 and 2,765,212, each of which is hereby incorporated by reference in its entirety. A talc is any soft metamorphic rock or soft silicate mineral and includes without limitation magnesium silicate and calcium silicate, see, e.g., U.S. Pat. No. 2,987,447, which is hereby incorporated by reference in its entirety.

In one embodiment, a single grip enhancer is present in a grip enhancing composition disclosed herein. In another embodiment, a plurality of grip enhancers is present in a grip enhancing composition disclosed herein. In aspects of this embodiment, a grip enhancing composition disclosed herein comprises, e.g., one or more grip enhancers, two or more grip enhancers, three or more grip enhancers, four or more grip enhancers or five or more grip enhancers. In other aspects of this embodiment, a grip enhancing composition disclosed herein comprises, e.g., only one grip enhancer, at most two grip enhancers, at most three grip enhancers, at most four grip enhancers, or at most five grip enhancers. In yet other aspects of this embodiment, a grip enhancing composition disclosed herein comprises from, e.g., 1 to 2 grip enhancers, 1 to 3 grip enhancers, 1 to 4 grip enhancers, 1 to 5 grip enhancers, 2 to 3 grip enhancers, 2 to 4 grip enhancers, 2 to 5 grip enhancers, 3 to 4 grip enhancers, 3 to 5 grip enhancers or 4 to 5 grip enhancers.

In another embodiment, a grip enhancing composition disclosed herein comprises an amount of grip enhancer that provides a desired beneficial effect to a grip enhancing composition disclosed herein. In aspects of this embodiment, a grip enhancing composition disclosed herein comprises a grip enhancer in an amount of, e.g., 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%. 9%, 10%, 15%, 20%, 25%, 30%, 35%, 40%, 45%, 50%, 55%, 60%, 65%, 70%, 75%, 80%, 85%, 90%, or 95% by weight of the composition. In other aspects of this embodiment, a grip enhancing composition disclosed herein comprises a grip enhancer in an amount of, e.g., at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% by weight of the composition. In yet other aspects of this embodiment, a grip enhancing composition disclosed herein comprises a grip enhancer in an amount of, e.g., at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, at most 10%, at most 15%, at most 20%, at most 25%, at most 30%, at most 35%, at most 40%, 40%, at most 45%, at most 50%, at most 55%, at most 60%, at most 65%, at most 70%, at most 75%, at most 80%, at most 85%, at most 90%, or at most 95% by weight of the composition.

In still other aspects of this embodiment, a grip enhancing composition disclosed herein comprises a grip enhancer in an amount of from, e.g., about 0.01% to about 0.05%, about 0.01% to about 0.075%, about 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 15%, about 0.1% to about 20%, about 0.1% to about 25%, about 0.1% to about 30%, about 0.1% to about 35%, about 0.1% to about 40%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.25% to about 15%, about 0.25% to about 20%, about 0.25% to about 25%, about 0.25% to about 30%, about 0.25% to about 35%, about 0.25% to about 40%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.5% to about 15%, about 0.5% to about 20%, about 0.5% to about 25%, about 0.5% to about 30%, about 0.5% to about 35%, about 0.5% to about 40%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 0.75% to about 15%, about 0.75% to about 20%, about 0.75% to about 25%, about 0.75% to about 30%, about 0.75% to about 35%, about 0.75% to about 40%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 15%, about 1% to about 20%, about 1% to about 25%, about 1% to about 30%, about 1% to about 35%, about 1% to about 40%, about 2% to about 3%, about 2% to about 4%, about 2% to about 5%, about 2% to about 6%, about 2% to about 7%, about 2% to about 8%, about 2% to about 9%, about 2% to about 10%, about 2% to about 15%, about 2% to about 20%, about 2% to about 25%, about 2% to about 30%, about 2% to about 35%, about 2% to about 40%, about 3% to about 4%, about 3% to about 5%, about 3% to about 6%, about 3% to about 7%, about 3% to about 8%, about 3% to about 9%, about 3% to about 10%, about 3% to about 15%, about 3% to about 20%, about 3% to about 25%, about 3% to about 30%, about 3% to about 35%, about 3% to about 40%, about 4% to about 5%, about 4% to about 6%, about 4% to about 7%, about 4% to about 8%, about 4% to about 9%, about 4% to about 10%, about 4% to about 15%, about 4% to about 20%, about 4% to about 25%, about 4% to about 30%, about 4% to about 35%, about 4% to about 40%, about 5% to about 6%, about 5% to about 7%, about 5% to about 8%, about 5% to about 9%, about 5% to about 10%, about 5% to about 15%, about 5% to about 20%, about 5% to about 25%, about 5% to about 30%, about 5% to about 35%, about 5% to about 40%, about 6% to about 7%, about 6% to about 8%, about 6% to about 9%, about 6% to about 10%, about 6% to about 15%, about 6% to about 20%, about 6% to about 25%, about 6% to about 30%, about 6% to about 35%, about 6% to about 40%, about 7% to about 8%, about 7% to about 9%, about 7% to about 10%, about 7% to about 15%, about 7% to about 20%, about 7% to about 25%, about 7% to about 30%, about 7% to about 35%, about 7% to about 40%, about 8% to about 9%, about 8% to about 10%, about 8% to about 15%, about 8% to about 20%, about 8% to about 25%, about 8% to about 30%, about 8% to about 35%, about 8% to about 40%, about 9% to about 10%, about 9% to about 15%, about 9% to about 20%, about 9% to about 25%, about 9% to about 30%, about 9% to about 35%, about 9% to about 40%, about 10% to about 15%, about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, or about 35% to about 40% by weight of the composition.

In other aspects of this embodiment, a grip enhancing composition disclosed herein comprises a grip enhancer in an amount of from, e.g., about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 20% to about 30%, about 20% to about 40%, about 20% to about 50%, about 20% to about 60%, about 20% to about 70%, about 20% to about 80%, about 20% to about 90%, about 20% to about 95%, about 30% to about 40%, about 30% to about 50%, about 30% to about 60%, about 30% to about 70%, about 30% to about 80%, about 30% to about 90%, about 30% to about 95%, about 40% to about 50%, about 40% to about 60%, about 40% to about 70%, about 40% to about 80%, about 40% to about 90%, about 40% to about 95%, about 50% to about 60%, about 50% to about 70%, about 50% to about 80%, about 50% to about 90%, about 50% to about 95%, about 60% to about 70%, about 60% to about 80%, about 60% to about 90%, about 60% to about 95%, about 70% to about 80%, about 70% to about 90%, about 70% to about 95%, about 80% to about 90%, about 80% to about 95%, or about 90% to about 95% by weight of the composition.

In another embodiment, a grip enhancing composition disclosed herein comprises an amount of chalk that provides a desired beneficial effect to a grip enhancing composition disclosed herein. In aspects of this embodiment, a grip enhancing composition disclosed herein comprises a chalk in an amount of, e.g., 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%. 9%, 10%, 15%, 20%, 25%, 30%, 35%, or 40% by weight of the composition. In other aspects of this embodiment, a grip enhancing composition disclosed herein comprises a chalk in an amount of, e.g., at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, or at least 40% by weight of the composition. In yet other aspects of this embodiment, a grip enhancing composition disclosed herein comprises a chalk in an amount of, e.g., at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, at most 10%, at most 15%, at most 20%, at most 25%, at most 30%, at most 35%, or at most 40% by weight of the composition.

In still other aspects of this embodiment, a grip enhancing composition disclosed herein comprises a chalk in an amount of from, e.g., 0.01% to about 0.05%, 0.01% to about 0.075%, 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 15%, about 0.1% to about 20%, about 0.1% to about 25%, about 0.1% to about 30%, about 0.1% to about 35%, about 0.1% to about 40%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.25% to about 15%, about 0.25% to about 20%, about 0.25% to about 25%, about 0.25% to about 30%, about 0.25% to about 35%, about 0.25% to about 40%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.5% to about 15%, about 0.5% to about 20%, about 0.5% to about 25%, about 0.5% to about 30%, about 0.5% to about 35%, about 0.5% to about 40%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 0.75% to about 15%, about 0.75% to about 20%, about 0.75% to about 25%, about 0.75% to about 30%, about 0.75% to about 35%, about 0.75% to about 40%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 15%, about 1% to about 20%, about 1% to about 25%, about 1% to about 30%, about 1% to about 35%, about 1% to about 40%, about 2% to about 3%, about 2% to about 4%, about 2% to about 5%, about 2% to about 6%, about 2% to about 7%, about 2% to about 8%, about 2% to about 9%, about 2% to about 10%, about 2% to about 15%, about 2% to about 20%, about 2% to about 25%, about 2% to about 30%, about 2% to about 35%, about 2% to about 40%, about 3% to about 4%, about 3% to about 5%, about 3% to about 6%, about 3% to about 7%, about 3% to about 8%, about 3% to about 9%, about 3% to about 10%, about 3% to about 15%, about 3% to about 20%, about 3% to about 25%, about 3% to about 30%, about 3% to about 35%, about 3% to about 40%, about 4% to about 5%, about 4% to about 6%, about 4% to about 7%, about 4% to about 8%, about 4% to about 9%, about 4% to about 10%, about 4% to about 15%, about 4% to about 20%, about 4% to about 25%, about 4% to about 30%, about 4% to about 35%, about 4% to about 40%, about 5% to about 6%, about 5% to about 7%, about 5% to about 8%, about 5% to about 9%, about 5% to about 10%, about 5% to about 15%, about 5% to about 20%, about 5% to about 25%, about 5% to about 30%, about 5% to about 35%, about 5% to about 40%, about 6% to about 7%, about 6% to about 8%, about 6% to about 9%, about 6% to about 10%, about 6% to about 15%, about 6% to about 20%, about 6% to about 25%, about 6% to about 30%, about 6% to about 35%, about 6% to about 40%, about 7% to about 8%, about 7% to about 9%, about 7% to about 10%, about 7% to about 15%, about 7% to about 20%, about 7% to about 25%, about 7% to about 30%, about 7% to about 35%, about 7% to about 40%, about 8% to about 9%, about 8% to about 10%, about 8% to about 15%, about 8% to about 20%, about 8% to about 25%, about 8% to about 30%, about 8% to about 35%, about 8% to about 40%, about 9% to about 10%, about 9% to about 15%, about 9% to about 20%, about 9% to about 25%, about 9% to about 30%, about 9% to about 35%, about 9% to about 40%, about 10% to about 15%, about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, or about 35% to about 40% by weight of the composition.

A grip enhancing composition disclosed herein may comprise, in part, a hydrophilic clay. A hydrophilic clay is a synthetic or naturally-occurring smectic clay that forms three-dimensional colloidal structures when hydrated leading to increased viscosity and improved suspension control components in a composition. A hydrophilic clay includes hydrous phyllosilicate clays. A hydrophilic clay can have thixotropic properties. Non-limiting examples of a hydrophilic clay include a bentonite clay, a hectorite clay, a kaolinite clay, and a silicate clay. A bentonite clay is a phyllosilicate clay and includes, without limitation, a potassium bentonite clay, a sodium bentonite clay, a calcium bentonite clay, and an aluminum bentonite clay. A hectorite clay is a phyllosilicate clay and includes those commercially sold as the BENTONE® product line, including hectorite clay (BENTONE® EW, hectorite clay (BENTONE® MA), and hectorite clay and hydroxy ethyl cellulose (BENTONE® LT). A kaolinite clay is a phyllosilicate clay. A silicate clay is a synthetic layered silicate clay and includes those commercially sold as the LAPONITE® product line, including lithium magnesium sodium silicate (LAPONITE® XLG), lithium magnesium sodium silicate and tetrasodium pyrophosphate (LAPONITE® XLS), sodium magnesium fluorosilicate (LAPONITE® XL), and sodium magnesium fluorosilicate (LAPONITE® XL21).

In one embodiment, a single hydrophilic clay is present in a grip enhancing composition disclosed herein. In another embodiment, a plurality of hydrophilic clays is present in a grip enhancing composition disclosed herein. In aspects of this embodiment, a grip enhancing composition disclosed herein comprises, e.g., one or more hydrophilic clays, two or more hydrophilic clays, three or more hydrophilic clays, four or more hydrophilic clays or five or more hydrophilic clays. In other aspects of this embodiment, a grip enhancing composition disclosed herein comprises, e.g., only one hydrophilic clay, at most two hydrophilic clays, at most three hydrophilic clays, at most four hydrophilic clays, or at most five hydrophilic clays. In yet other aspects of this embodiment, a grip enhancing composition disclosed herein comprises from, e.g., 1 to 2 hydrophilic clays, 1 to 3 hydrophilic clays, 1 to 4 hydrophilic clays, 1 to 5 hydrophilic clays, 2 to 3 hydrophilic clays, 2 to 4 hydrophilic clays, 2 to 5 hydrophilic clays, 3 to 4 hydrophilic clays, 3 to 5 hydrophilic clays or 4 to 5 hydrophilic clays.

In another embodiment, a grip enhancing composition disclosed herein comprises an amount of a hydrophilic clay that provides a desired beneficial effect to a grip enhancing composition disclosed herein. In aspects of this embodiment, a composition disclosed herein comprises a hydrophilic clay in an amount of, e.g., 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%. 9%, or 10% by weight of the composition. In other aspects of this embodiment, a grip enhancing composition disclosed herein comprises a hydrophilic clay in an amount of, e.g., at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, or at least 10% by weight of the composition. In yet other aspects of this embodiment, a grip enhancing composition disclosed herein comprises a hydrophilic clay in an amount of, e.g., at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, or at most 10% by weight of the composition.

In still other aspects of this embodiment, a grip enhancing composition disclosed herein comprises a hydrophilic clay in an amount of from, e.g., about 0.01% to about 0.05%, 0.01% to about 0.075%, about 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10% or about 9% to about 10% by weight of the composition.

In another embodiment, a grip enhancing composition disclosed herein comprises an amount of a silicate clay that provides a desired beneficial effect to a grip enhancing composition disclosed herein. In aspects of this embodiment, a grip enhancing composition disclosed herein comprises a silicate clay in an amount of, e.g., 0.001%, 0.0025%, 0.005%, 0.0075%, 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%. 9%, or 10% by weight of the composition. In other aspects of this embodiment, a grip enhancing composition disclosed herein comprises a silicate clay in an amount of, e.g., at least 0.001%, at least 0.0025%, at least 0.005%, at least 0.0075%, at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, or at least 10% by weight of the composition. In yet other aspects of this embodiment, a grip enhancing composition disclosed herein comprises a silicate clay in an amount of, e.g., at most 0.001%, at most 0.0025%, at most 0.005%, at most 0.0075%, at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, or at most 10% by weight of the composition.

In still other aspects of this embodiment, a grip enhancing composition disclosed herein comprises a silicate clay in an amount of from, e.g., about 0.001% to about 0.005%, 0.001% to about 0.0075%, about 0.001% to about 0.01%, about 0.001% to about 0.05%, about 0.001% to about 0.075%, about 0.001% to about 0.1%, about 0.001% to about 0.25%, about 0.001% to about 0.5%, about 0.001% to about 0.75%, about 0.005% to about 0.0075%, about 0.005% to about 0.01%, about 0.005% to about 0.05%, about 0.005% to about 0.075%, about 0.005% to about 0.1%, about 0.005% to about 0.25%, about 0.005% to about 0.5%, about 0.005% to about 0.75%, about 0.01% to about 0.05%, 0.01% to about 0.075%, about 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10% or about 9% to about 10% by weight of the composition.

In one embodiment, a grip enhancing composition disclosed herein does not comprise a hydrophilic clay. In one embodiment, a grip enhancing composition disclosed herein does not comprise a silicate clay.

A grip enhancing composition disclosed herein may comprise, in part, a polysaccharide. Polysaccharides are polymeric carbohydrate molecules composed of long chains of monosaccharide units bound together by glycosidic linkages. As a rule of thumb, polysaccharides contain more than ten monosaccharide units, whereas oligosaccharides contain three to ten monosaccharide units; but the precise cutoff varies somewhat according to convention. Polysaccharides useful herein form three-dimensional colloidal structures when hydrated leading to increased viscosity and improved suspension control components in a composition. A polysaccharide includes cellulose, a cellulose ether derivative, a cellulose ester derivative, any salt thereof, or any combination thereof. Non-limiting examples of a cellulose ether derivative include ethyl cellulose, methyl cellulose, ethylmethyl cellulose, hydroxyethyl cellulose, hydroxypropyl cellulose, hydroxyethylmethyl cellulose, hydroxypropylmethyl cellulose, ethyl hydroxyethyl cellulose, carboxymethyl cellulose, or any salt thereof. Non-limiting examples of a cellulose ester derivative include cellulose acetate, cellulose acetate phthalate, cellulose acetate butyrate, cellulose acetate trimelitate, hydroxupropylmethyl cellulose phthalate, or any salt thereof. Additional cellulose derivatives are described in, e.g., CELLULOSE: STRUCTURE AND PROPERTIES, DERIVATIVES AND INDUSTRIAL USES, eds. Lejeune and Deprez, pp. 528 (2010); CELLULOSE AND CELLULOSE DERIVATIVES, Kenji Kamide, (2005); Doelker, Cellulose Derivatives, pp. 199-265, ADVANCES IN POLYMER SCIENCE, vol. 107 (2005), each of which is hereby incorporated by reference in its entirety.

In one embodiment, a single polysaccharide is present in a grip enhancing composition disclosed herein. In another embodiment, a plurality of polysaccharides is present in a grip enhancing composition disclosed herein. In aspects of this embodiment, a grip enhancing composition disclosed herein comprises, e.g., one or more polysaccharides, two or more polysaccharides, three or more polysaccharides, four or more polysaccharides or five or more polysaccharides. In other aspects of this embodiment, a grip enhancing composition disclosed herein comprises, e.g., only one polysaccharide, at most two polysaccharides, at most three polysaccharides, at most four polysaccharides, or at most five polysaccharides. In yet other aspects of this embodiment, a grip enhancing composition disclosed herein comprises from, e.g., 1 to 2 polysaccharides, 1 to 3 polysaccharides, 1 to 4 polysaccharides, 1 to 5 polysaccharides, 2 to 3 polysaccharides, 2 to 4 polysaccharides, 2 to 5 polysaccharides, 3 to 4 polysaccharides, 3 to 5 polysaccharides or 4 to 5 polysaccharides.

In another embodiment, a grip enhancing composition disclosed herein comprises an amount of a polysaccharide that provides a desired beneficial effect to a grip enhancing composition disclosed herein. In aspects of this embodiment, a composition disclosed herein comprises a polysaccharide in an amount of, e.g., 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%. 9%, or 10% by weight of the composition. In other aspects of this embodiment, a grip enhancing composition disclosed herein comprises a polysaccharide in an amount of, e.g., at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, or at least 10% by weight of the composition. In yet other aspects of this embodiment, a grip enhancing composition disclosed herein comprises a polysaccharide in an amount of, e.g., at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, or at most 10% by weight of the composition.

In still other aspects of this embodiment, a grip enhancing composition disclosed herein comprises a polysaccharide in an amount of from, e.g., about 0.01% to about 0.05%, 0.01% to about 0.075%, about 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10% or about 9% to about 10% by weight of the composition.

In another embodiment, a grip enhancing composition disclosed herein comprises an amount of a cellulose ether that provides a desired beneficial effect to a grip enhancing composition disclosed herein. In aspects of this embodiment, a grip enhancing composition disclosed herein comprises a cellulose ether in an amount of, e.g., 0.001%, 0.0025%, 0.005%, 0.0075%, 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%. 9%, or 10% by weight of the composition. In other aspects of this embodiment, a grip enhancing composition disclosed herein comprises a cellulose ether in an amount of, e.g., at least 0.001%, at least 0.0025%, at least 0.005%, at least 0.0075%, at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, or at least 10% by weight of the composition. In yet other aspects of this embodiment, a grip enhancing composition disclosed herein comprises a cellulose ether in an amount of, e.g., at most 0.001%, at most 0.0025%, at most 0.005%, at most 0.0075%, at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, or at most 10% by weight of the composition.

In still other aspects of this embodiment, a grip enhancing composition disclosed herein comprises a cellulose ether in an amount of from, e.g., about 0.001% to about 0.005%, 0.001% to about 0.0075%, about 0.001% to about 0.01%, about 0.001% to about 0.05%, about 0.001% to about 0.075%, about 0.001% to about 0.1%, about 0.001% to about 0.25%, about 0.001% to about 0.5%, about 0.001% to about 0.75%, about 0.005% to about 0.0075%, about 0.005% to about 0.01%, about 0.005% to about 0.05%, about 0.005% to about 0.075%, about 0.005% to about 0.1%, about 0.005% to about 0.25%, about 0.005% to about 0.5%, about 0.005% to about 0.75%, about 0.01% to about 0.05%, 0.01% to about 0.075%, about 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10% or about 9% to about 10% by weight of the composition.

In one embodiment, a grip enhancing composition disclosed herein does not comprise a polysaccharide. In one embodiment, a grip enhancing composition disclosed herein does not comprise a cellulose ether.

A grip enhancing composition disclosed herein may optionally comprise, in part, a silica. Silica, also known as silicon dioxide, is an oxide of silicon and has the chemical formula SiO₂. Silica is useful for its light-diffusing properties and natural absorbency.

The amount of silica used in a grip enhancing composition disclosed herein is an amount that provides a desired beneficial effect to a grip enhancing composition disclosed herein. In aspects of this embodiment, a grip enhancing composition disclosed herein comprises a silica in an amount of, e.g., 0.001%, 0.0025%, 0.005%, 0.0075%, 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%. 9%, 10%, 15%, 20%, 25%, 30%, 35%, or 40% by weight of the composition. In other aspects of this embodiment, a grip enhancing composition disclosed herein comprises a silica in an amount of, e.g., at least 0.001%, at least 0.0025%, at least 0.005%, at least 0.0075%, at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, or at least 40% by weight of the composition. In yet other aspects of this embodiment, a grip enhancing composition disclosed herein comprises a silica in an amount of, e.g., at most 0.001%, at most 0.0025%, at most 0.005%, at most 0.0075%, at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, at most 10%, at most 15%, at most 20%, at most 25%, at most 30%, at most 35%, or at most 40% by weight of the composition.

In still other aspects of this embodiment, a grip enhancing composition disclosed herein comprises a silica in an amount of from, e.g., about 0.001% to about 0.005%, 0.001% to about 0.0075%, about 0.001% to about 0.01%, about 0.001% to about 0.05%, about 0.001% to about 0.075%, about 0.001% to about 0.1%, about 0.001% to about 0.25%, about 0.001% to about 0.5%, about 0.001% to about 0.75%, about 0.005% to about 0.0075%, about 0.005% to about 0.01%, about 0.005% to about 0.05%, about 0.005% to about 0.075%, about 0.005% to about 0.1%, about 0.005% to about 0.25%, about 0.005% to about 0.5%, about 0.005% to about 0.75%, about 0.01% to about 0.05%, 0.01% to about 0.075%, about 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 15%, about 0.1% to about 20%, about 0.1% to about 25%, about 0.1% to about 30%, about 0.1% to about 35%, about 0.1% to about 40%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.25% to about 15%, about 0.25% to about 20%, about 0.25% to about 25%, about 0.25% to about 30%, about 0.25% to about 35%, about 0.25% to about 40%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.5% to about 15%, about 0.5% to about 20%, about 0.5% to about 25%, about 0.5% to about 30%, about 0.5% to about 35%, about 0.5% to about 40%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 0.75% to about 15%, about 0.75% to about 20%, about 0.75% to about 25%, about 0.75% to about 30%, about 0.75% to about 35%, about 0.75% to about 40%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 15%, about 1% to about 20%, about 1% to about 25%, about 1% to about 30%, about 1% to about 35%, about 1% to about 40%, about 2% to about 3%, about 2% to about 4%, about 2% to about 5%, about 2% to about 6%, about 2% to about 7%, about 2% to about 8%, about 2% to about 9%, about 2% to about 10%, about 2% to about 15%, about 2% to about 20%, about 2% to about 25%, about 2% to about 30%, about 2% to about 35%, about 2% to about 40%, about 3% to about 4%, about 3% to about 5%, about 3% to about 6%, about 3% to about 7%, about 3% to about 8%, about 3% to about 9%, about 3% to about 10%, about 3% to about 15%, about 3% to about 20%, about 3% to about 25%, about 3% to about 30%, about 3% to about 35%, about 3% to about 40%, about 4% to about 5%, about 4% to about 6%, about 4% to about 7%, about 4% to about 8%, about 4% to about 9%, about 4% to about 10%, about 4% to about 15%, about 4% to about 20%, about 4% to about 25%, about 4% to about 30%, about 4% to about 35%, about 4% to about 40%, about 5% to about 6%, about 5% to about 7%, about 5% to about 8%, about 5% to about 9%, about 5% to about 10%, about 5% to about 15%, about 5% to about 20%, about 5% to about 25%, about 5% to about 30%, about 5% to about 35%, about 5% to about 40%, about 6% to about 7%, about 6% to about 8%, about 6% to about 9%, about 6% to about 10%, about 6% to about 15%, about 6% to about 20%, about 6% to about 25%, about 6% to about 30%, about 6% to about 35%, about 6% to about 40%, about 7% to about 8%, about 7% to about 9%, about 7% to about 10%, about 7% to about 15%, about 7% to about 20%, about 7% to about 25%, about 7% to about 30%, about 7% to about 35%, about 7% to about 40%, about 8% to about 9%, about 8% to about 10%, about 8% to about 15%, about 8% to about 20%, about 8% to about 25%, about 8% to about 30%, about 8% to about 35%, about 8% to about 40%, about 9% to about 10%, about 9% to about 15%, about 9% to about 20%, about 9% to about 25%, about 9% to about 30%, about 9% to about 35%, about 9% to about 40%, about 10% to about 15%, about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, or about 35% to about 40% by weight of the composition.

A grip enhancing composition disclosed herein may optionally comprise, in part, a hypochlorous acid. A weak acid, the chemical formula of hypochlorous acid is HOCI, while its molecular formula is written as HCIO. As shown in formula I, hypochlorous acid is a simple molecule with the central oxygen connected to chlorine and hydrogen atoms through single bonds and has molar mass is 52.46 g/mol.

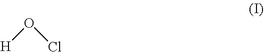

Hypochlorous acid is a colorless solution, and its exact physical properties are variable, depending on the concentration of hypochlorous in solution. Hypochlorous acid reacts with bases to form salts called hypochlorites. For example, sodium hypochlorite (NaOCl), the active ingredient in bleach, is formed by reacting hypochlorous acid with sodium hydroxide. Hypochlorous acid also readily reacts with a variety of organic molecules and biomolecules.

The hypochlorous acid solution can be produced, e.g., by dissolving chlorine in water, hydrolysis of chlorine gas, electrolysis of a salt solution or acidification of hypochloride. For example, stable hypochlorous salts, such as, e.g., alkali metal hypochlorites like sodium hypochlorite, calcium hypochlorite, potassium hypochlorite, lithium hypochlorite and magnesium hypochlorite, can be obtained by dissolving chlorine gas into an aqueous alkali metal hydroxide solution, like a sodium hydroxide solution, a calcium hydroxide solution, a potassium hydroxide solution, a lithium hydroxide solution, or a magnesium hydroxide solution. Hypochlorous acid can also be prepared by dissolving monoxide in water. As another example, hypochlorous acid can also be produced by electrolytically treating a saline solution. In one method, an electrical current is applied to a three-compartment cell comprising a cathode chamber, an anode chamber, and a central saline solution chamber interposed between the other two chambers where each chamber is separated by a semi-permeable membrane. During electrolysis, sodium chloride (NaCl) dissociates into negatively charged chloride (Cl⁻) and positively charged sodium (Na⁺). At the same time, water dissociates into hydroxide (OH−) and hydrogen (H⁺) ions are formed. The negatively charged chloride (Cl⁻) and hydroxide (OH⁻) ions move to the anode to lose electrons and form hypochlorous acid (HOCl) as well as hypochlorite ions (OH⁻) and oxygen (O₂) and chlorine (Cl₂) gases. The reductive water comprising the hypochlorous acid is then dispensed into a collection chamber for subsequent use. Methods to produce hypochlorous acid are described in, e.g., U.S. Pat. Nos. 3,914,397, 4,190,638, 4,908,215, 5,322,677, 6,426,066, 7,323,118, 8,062,500, 8,945,630, 9,168,318, and 9,486,479, each of which is hereby incorporated by reference in its entirety.

In an embodiment, a grip enhancing composition disclosed herein comprises an amount of hypochlorous acid that provides a desired beneficial effect to a composition disclosed herein. In aspects of this embodiment, a grip enhancing composition disclosed herein comprises hypochlorous acid in an amount of, e.g., 0.00005%, 0.00006%, 0.00007%, 0.000075%, 0.00008%, 0.00009%, 0.0001%, 0.0005%, 0.001%, 0.0015%, 0.002%, 0.0025%, 0.003%, 0.0035%, 0.004%, 0.0045%, 0.005%, 0.0055%, 0.006%, 0.007%, 0.0075%, 0.008%, 0.009%, 0.01%, 0.02%, 0.03%, 0.04% or 0.05% by weight of the composition. In other aspects of this embodiment, a grip enhancing composition disclosed herein comprises hypochlorous acid in an amount of, e.g., at least 0.00005%, at least 0.00006%, at least 0.00007%, at least 0.000075%, at least 0.00008%, at least 0.00009%, at least 0.0001%, at least 0.0005%, at least 0.001%, at least 0.0015%, at least 0.002%, at least 0.0025%, at least 0.003%, at least 0.0035%, at least 0.004%, at least 0.0045%, at least 0.005%, at least 0.0055%, at least 0.006%, at least 0.007%, at least 0.0075%, at least 0.008%, at least 0.009%, at least 0.01%, at least 0.02%, at least 0.03%, at least 0.04% or at least 0.05% by weight of the composition. In yet other aspects of this embodiment, a grip enhancing composition disclosed herein comprises hypochlorous acid in an amount of, e.g., at most 0.00005%, at most 0.00006%, at most 0.00007%, at most 0.000075%, at most 0.00008%, at most 0.00009%, at most 0.0001%, at most 0.0005%, at most 0.001%, at most 0.0015%, at most 0.002%, at most 0.0025%, at most 0.003%, at most 0.0035%, at most 0.004%, at most 0.0045%, at most 0.005%, at most 0.0055%, at most 0.006%, at most 0.007%, at most 0.0075%, at most 0.008%, at most 0.009%, at most 0.01%, at most 0.02%, at most 0.03%, at most 0.04% or at most 0.05% by weight of the composition. In still other aspects of this embodiment, a grip enhancing composition disclosed herein comprises hypochlorous acid in an amount of from, e.g., about 0.00075% to about 0.002%, about 0.00075% to about 0.003%, about 0.00075% to about 0.004%, about 0.00075% to about 0.005%, about 0.00075% to about 0.006%, about 0.00075% to about 0.007%, about 0.00075% to about 0.008%, about 0.00075% to about 0.009%, about 0.00075% to about 0.01%, about 0.001% to about 0.002%, about 0.001% to about 0.003%, about 0.001% to about 0.004%, about 0.001% to about 0.005%, about 0.001% to about 0.006%, about 0.001% to about 0.007%, about 0.001% to about 0.008%, about 0.001% to about 0.009%, about 0.001% to about 0.01%, about 0.002% to about 0.003%, about 0.002% to about 0.004%, about 0.002% to about 0.005%, about 0.002% to about 0.006%, about 0.002% to about 0.007%, about 0.002% to about 0.008%, about 0.002% to about 0.009%, or about 0.002% to about 0.01% by weight of the composition.

In aspects of this embodiment, a grip enhancing composition disclosed herein comprises hypochlorous acid in an amount of, e.g., 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm or 400 ppm. In other aspects of this embodiment, a grip enhancing composition disclosed herein comprises hypochlorous acid in an amount of, e.g., at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm or at least 400 ppm. In yet other aspects of this embodiment, a grip enhancing composition disclosed herein comprises hypochlorous acid in an amount of, e.g., at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm or at most 400 ppm.

In still other aspects of this embodiment, a grip enhancing composition disclosed herein comprises hypochlorous acid in an amount of from, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a grip enhancing composition disclosed herein comprises hypochlorous acid in an amount of from, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.

In one embodiment, a grip enhancing composition disclosed herein does not comprise hypochlorous acid.

The concentration of hypochlorous acid in solution may be described as free available chlorine in parts per million. Hypochlorous acid is in equilibrium with hypochlorite ions (OCl⁻) and dissolved chlorine gas (Cl₂). The extent of the equilibrium is determined predominately by the pH of the solution. Temperature also impacts the ratio of the free chlorine component. Therefore, both FAC and pH need to be known to understand the amount of chlorine present as hypochlorous acid. In general, when the pH range is about 4.0 to about 5.6, approximately 100% of the available chlorine is present as HOCI. As the pH is lowered below about 4, there is an increase in dissolved chlorine gas (Cl₂). Thus, at a pH of about 3, about 90% of the available chlorine is present as hypochlorous acid, at a pH of about 2, about 75% of the available chlorine is present as hypochlorous acid, at a pH of about 1.5, about 50% of the available chlorine is present as hypochlorous acid, while at a pH of about 1, about 25% of the available chlorine is present as hypochlorous acid. As the pH is increase above about 5.6, there is an increase in hypochlorite ions (OCl⁻). Thus, at a pH of about 6.5, about 90% of the available chlorine is present as hypochlorous acid, at a pH of about 7, about 75% of the available chlorine is present as hypochlorous acid, at a pH of about 7.5, about 50% of the available chlorine is present as hypochlorous acid, while at a pH of about 8, about 25% of the available chlorine is present as hypochlorous acid.

The chlorine amount may be measured by methods known in the art, such as the DPD colorimeter method (Lamotte Company, Chestertown, Md.) or other known methods established by the Environmental Protection Agency. In the DPD colorimeter method, a yellow color is formed by the reaction of free chlorine With N,N-diethyl-p-phenylenediamine (DPD) and the intensity is measured With a calibrated calorimeter that provides the output in parts per million. Further addition of potassium iodide turns the solution a pink color to provide the total chlorine value.

In aspects of this embodiment, a grip enhancing composition disclosed herein comprises free available chlorine in an amount of, e.g., 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm or 400 ppm. In other aspects of this embodiment, a grip enhancing composition disclosed herein comprises free available chlorine in an amount of, e.g., at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm or at least 400 ppm. In yet other aspects of this embodiment, a grip enhancing composition disclosed herein comprises free available chlorine in an amount of, e.g., at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm or at most 400 ppm.

In still other aspects of this embodiment, a grip enhancing composition disclosed herein comprises free available chlorine in an amount of from, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a grip enhancing composition disclosed herein comprises free available chlorine in an amount of from, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.

In one embodiment, a grip enhancing composition disclosed herein does not comprise free available chlorine.

A grip enhancing composition disclosed herein may optionally comprise, in part, electrolytic alkaline water. Electrolytic alkaline water is produces using a process called electrolysis. This process is characterized by utilizing electric current to electrolyze a salt solution. This process will yield solution of potassium hydroxide (KOH) or sodium hydroxide (NaOH) as alkaline product depending on the type of salt used. This process allows transporting the hydroxide ions (OH−) from one chamber to the other. In one embodiment, electrolytic alkaline water is used as a replacement to water and in lieu of hypochlorous acid and/or free available chlorine. A grip enhancing composition disclosed herein produced using electrolytic alkaline water typically has an unadjusted pH of about 10.0 to 12.0.

In one embodiment, a grip enhancing composition disclosed herein does not comprise electrolytic alkaline water.

A grip enhancing composition disclosed herein may optionally comprise an ascorbic acid. An antioxidant, an ascorbic acid is also known as Vitamin C. An ascorbic acid disclosed herein can be in a powdered or other solid form or a liquid form, e.g., an oil-based solution. Ascorbic acid is incompatible with hypochlorous acid and/or free available chorine. As such, if a grip enhancing composition disclosed herein contains hypochlorous acid and/or free available chlorine, ascorbic acid is excluded from the composition. Similarly, if a grip enhancing composition disclosed herein contains ascorbic acid, hypochlorous acid, free available chlorine, or both hypochlorous acid and free available chlorine are excluded from the composition.

In an embodiment, a grip enhancing composition disclosed herein comprises an amount of ascorbic acid that provides a desired beneficial effect to a composition disclosed herein. In aspects of this embodiment, a grip enhancing composition disclosed herein comprises ascorbic acid in an amount of, e.g., 0.00005%, 0.00006%, 0.00007%, 0.000075%, 0.00008%, 0.00009%, 0.0001%, 0.0005%, 0.001%, 0.0015%, 0.002%, 0.0025%, 0.003%, 0.0035%, 0.004%, 0.0045%, 0.005%, 0.0055%, 0.006%, 0.007%, 0.0075%, 0.008%, 0.009%, 0.01%, 0.02%, 0.03%, 0.04% or 0.05% by weight of the composition. In other aspects of this embodiment, a grip enhancing composition disclosed herein comprises ascorbic acid in an amount of, e.g., at least 0.00005%, at least 0.00006%, at least 0.00007%, at least 0.000075%, at least 0.00008%, at least 0.00009%, at least 0.0001%, at least 0.0005%, at least 0.001%, at least 0.0015%, at least 0.002%, at least 0.0025%, at least 0.003%, at least 0.0035%, at least 0.004%, at least 0.0045%, at least 0.005%, at least 0.0055%, at least 0.006%, at least 0.007%, at least 0.0075%, at least 0.008%, at least 0.009%, at least 0.01%, at least 0.02%, at least 0.03%, at least 0.04% or at least 0.05% by weight of the composition. In yet other aspects of this embodiment, a grip enhancing composition disclosed herein comprises ascorbic acid in an amount of, e.g., at most 0.00005%, at most 0.00006%, at most 0.00007%, at most 0.000075%, at most 0.00008%, at most 0.00009%, at most 0.0001%, at most 0.0005%, at most 0.001%, at most 0.0015%, at most 0.002%, at most 0.0025%, at most 0.003%, at most 0.0035%, at most 0.004%, at most 0.0045%, at most 0.005%, at most 0.0055%, at most 0.006%, at most 0.007%, at most 0.0075%, at most 0.008%, at most 0.009%, at most 0.01%, at most 0.02%, at most 0.03%, at most 0.04% or at most 0.05% by weight of the composition. In still other aspects of this embodiment, a grip enhancing composition disclosed herein comprises ascorbic acid in an amount of from, e.g., about 0.00075% to about 0.002%, about 0.00075% to about 0.003%, about 0.00075% to about 0.004%, about 0.00075% to about 0.005%, about 0.00075% to about 0.006%, about 0.00075% to about 0.007%, about 0.00075% to about 0.008%, about 0.00075% to about 0.009%, about 0.00075% to about 0.01%, about 0.001% to about 0.002%, about 0.001% to about 0.003%, about 0.001% to about 0.004%, about 0.001% to about 0.005%, about 0.001% to about 0.006%, about 0.001% to about 0.007%, about 0.001% to about 0.008%, about 0.001% to about 0.009%, about 0.001% to about 0.01%, about 0.002% to about 0.003%, about 0.002% to about 0.004%, about 0.002% to about 0.005%, about 0.002% to about 0.006%, about 0.002% to about 0.007%, about 0.002% to about 0.008%, about 0.002% to about 0.009%, or about 0.002% to about 0.01% by weight of the composition.

In aspects of this embodiment, a grip enhancing composition disclosed herein comprises ascorbic acid in an amount of, e.g., 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm or 400 ppm. In other aspects of this embodiment, a grip enhancing composition disclosed herein comprises ascorbic acid in an amount of, e.g., at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm or at least 400 ppm. In yet other aspects of this embodiment, a grip enhancing composition disclosed herein comprises ascorbic add in an amount of, e.g., at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm or at most 400 ppm.

In still other aspects of this embodiment, a grip enhancing composition disclosed herein comprises ascorbic acid in an amount of from, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a grip enhancing composition disclosed herein comprises ascorbic, acid in an amount of from, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.

In one embodiment, a grip enhancing composition disclosed herein does not comprise ascorbic acid.

A grip enhancing composition disclosed herein may optionally comprise, in part, an antimicrobial agent. An antimicrobial agent is one that kill microorganisms or inhibits their growth. An antimicrobial agent can be classified according to the microorganism it acts primarily against. For example, an antibacterial agent is an antimicrobial agent used against bacteria while an antifungal agent is an antimicrobial agent used against a fungus. An antimicrobial can be classified according it function. For example, an antimicrobial agent that kill microbes is called a microbicidal agent, while one that inhibits microbial growth are called biostatic agent. An antimicrobial agent can be classified according to its mode of action. For example, a disinfectant is an antimicrobial agent that kill a wide range of microorganisms on non-living surfaces to prevent the spread of illness, an antiseptic agent is an antimicrobial agent that kill a wide range of microorganisms on living tissue and help reduce infection, while an antibiotic agent is an antimicrobial agent that kills microorganisms within a body.

Specific antifungals or microbial agents with antifungal actives include, without limitation, β-lactam drugs, quinolone drugs, 2,4,4′-trichloro-2′-hydroxy diphenyl ether, 3,4,4′-trichlorocarbanilide, amanfadine, amanfadine hydrochloride, amanfadine sulfate, amikacin, amikacin sulfate, benzoic acid, butenafine, butocouazole nitrate, capreomycin, capreomycin sulfate, chlorhexidine, chlorhexidine gluconate, chlorhexidine hydrochloride, chlortetracycline, chlortetracycline hydrochloride, ciclopirox, ciprofloxacin, clindamycin hydrochloride, clotrimazole, doxycycline, doxycycline hydrochloride, econazole, efinaconazole, erythromycin, erythromycin estolate, erythromycin stearate, ethambutol, ethambutol hydrochloride, gentamicin, gentamicin sulfate, haloprogin, hexamidine isethionate, kanamycin, kanamycin sulfate, ketoconazole, lineomycin, lineomycin hydrochloride, luliconazole, methacycline, methacycline hydrochloride, methenamine, methenamine hippurate, methenamine mandelate, metronidazole, metronidazole hydrochloride, miconazole, miconazole hydrochloride, minocycline, minocycline hydrochloride, naftifine, neomycin, neomycin sulfate, netilmicin, netilmicin sulfate, norfloxacin, nystatin, octopirox, oxiconazole, oxytetracycline, parachlorometa xylenol, paromomycin, paromomycin sulfate, phenoxyethanol, phenoxy propanol, phenoxyisopropanol, pentamidine, pentamidine hydrochloride, paromomycin, salicylic acid, sertaconazole, streptomycin, streptomycin sulfate, sulconazole, tavaborole, terbinafine, tetracycline, tetracycline hydrochloride, tobramycin, tobramycin sulfate, tolnaftate, undecylenic acid, zinc oxytetracycline hydrochloride and zinc pyrithione

Antibacterials include, without limitation, an aminoglycoside, a tetracycline, a glycylcycline, a fluorocycline, an oxazolidinone, a peptidyl transferase (like an amphenicol and a pleuromutilin), a macrolide, a lincosamide, a streptogramin, a steroid antibacterial, a β-lactam (like a penicillin, a penem, a carbapenem, a cephem, a monobactam and a 3-lactamase inhibitor), an antifolates (like a dihydrofolate reductase (DHFR) inhibitor, a sulphonamide, a topoisomerase inhibitor and a quinolone), an anaerobic DNA inhibitor (like a nitroimidazole derivative, a nitrofuran derivative and a rifamycin).

Antiseptics include, without limitation, an acridine compound (like ethacridine lactate, 9-Aminoacridine and euflavine), a biguanide compound, an amidine compound (like 1,8-Diazabicyco[5.4.0]Undec-7-ene (DBU), diminazene, and benzamidine, chlorhexidine, dibrompropamidine, propamidine and hexamidine), a phenol compound (like hexachlorophene, policresulen, phenol, triclosan, triclocarban, chloroxylenol, biphenylol and fenticlor), a nitrofuran compound (like nitrofurazone), an iodine compound (iodine/octylphenoxypolyglycolether, povidone-iodine, and diiodohydroxypropane), a quinoline compound (like dequalinium, chlorquinaldol, qxyquinoline and clioquinol), a quaternary ammonium compound (like benzalkonium, benzethonium chloride, betrimonium, cetylpyridinium, cetrimide, benzoxonium chloride and didecyldimethylammonium chloride), a mercurial compound (like mercuric amidochloride, phenylmercuric borate, mercuric chloride, merbromin, thiomersal, mercuric iodide), a silver compound (like silver nitrate), an alcohol (like propanol, isopropanol, ethanol and other antiseptics like potassium permanganate, sodium hypochlorite, hydrogen peroxide, eosin, tosylchloramide and octenidine dihydrochloride.

Other antibacterial and antiseptic compounds include, without limitation, butocouazole phenol-TEA complex, mupirocin, triclosan, chlorocresol, chlorbutol, iodine, clindamycin, CAE (Anjinomoto Co., Inc., containing DL-pyrrolidone carboxylic acid salt of L-Cocoyl Arginine Ethyl Ester), povidone-iodine, polyhexanide, polyhexamethylene biguanide, polymyxin b sulfate-bacitracin, zinc-neomycin sulfate-hydrocortisone, chloramphenicol, methylbenzethonium chloride, manuka honey and erythromycin and antiseptics (e.g., benzalkonium chloride, benzethonium chloride, chlorhexidine gluconate, mafenide acetate, nitrofurazone, nitromersol), antimicrobial deodorant compounds, antiparasitics, including, without limitation, lindane and the like may be included in a grip enhancing composition disclosed herein.

Additional antimicrobial agents are described in, e.g. U.S. Pat. Nos. 6,827,795; 6,517,854; 6,010,817; 5,173,216; 5,719,113; 5,259,984; 5,562,912; 5,629,006; 5,728,662; 5,767,163; 5,750,579; 5,591,442; 5,650,143; 5,772,640; and 4,478,821, each of which is hereby incorporated by reference in its entirety.

In one embodiment, a grip enhancing composition disclosed herein does not comprise ozone. In another embodiment, a grip enhancing composition disclosed herein can comprise ozone.

In one embodiment, a grip enhancing composition disclosed herein does not comprise hydrogen peroxide. In another embodiment, a grip enhancing composition disclosed herein can comprise hydrogen peroxide.

In one embodiment, a grip enhancing composition disclosed herein does not comprise dissolved inorganic carbon, which can form a bicarbonate or carbonate of an alkali or alkaline earth metal. In another embodiment, a grip enhancing composition disclosed herein can comprise dissolved inorganic carbon, which can form a bicarbonate or carbonate of an alkali or alkaline earth metal.

In one embodiment, a grip enhancing composition disclosed herein does not comprise sodium dihydrogen phosphate. In another embodiment, a grip enhancing composition disclosed herein can comprise sodium dihydrogen phosphate.

A grip enhancing composition disclosed herein may optionally comprise, in part, a fragrance. A fragrance is a compound sufficiently volatile to be transported to the olfactory system in the upper part of a nose where it can trigger a smell or odor. A fragrance is also known as an odorant, an aroma, or a scent and can produced synthetically or be part of, or extracted from, e.g., a food, a wine, a spice, a herbal distillate, or a fragrance oil. A fragrance can be classified by structure, such as, e.g., an aldehyde-based fragrance, an amine-based fragrance, an aromatic-based fragrance, an ester-based fragrance, a ketone-based fragrance, a lactone-based fragrance, a thiol-based fragrance, or a terpene-based fragrance. Non-limiting examples of an aldehyde-based fragrance include acetaldehyde, hexanal, cis-3-hexenal, furfural, hexyl cinnamaldehyde, isovaleraldehyde, anisic aldehyde and cuminaldehyde. Non-limiting examples of an amine-based fragrance include triethylamine, putrescine, cadaverine, pyridine, indole and skatole. Non-limiting examples of an aromatic-based fragrance include benzaldehyde, eugenol, cinnamaldehyde, ethyl maltol, vanillin, anisole, anethole, estragole, and thymol. Non-limiting examples of an ester-based fragrance include fructone, geranyl acetate, methyl formate, methyl acetate, methyl propionate, methyl butyrate, ethyl acetate, ethyl butyrate, ethyl methylphenylglycidate, isoamyl acetate, pentyl butyrate, pentyl pentanoate, hexyl acetate, octyl acetate, benzyl acetate, and methyl anthranilate. Non-limiting examples of a ketone-based fragrance include cyclopentadecanone, dihydrojasmone, oct-1-en-3-one, 2-acetyl-1-pyrroline, and 6-acetyl-2,3,4,5-tetrahydropyrine. Non-limiting examples of a lactone-based fragrance include γ-decalactone, γ-nonalactone, δ-octalactone, jasmine lactone, massoia lactone, wine lactone and sotolon. Non-limiting examples of a thiol-based fragrance include thioacetone, allyl thiol, ethanethiol, 2-methyl-2-propanethiol, butane-1-thiol, grapefruit mercaptan, methanethiol, furan-2-ylmethanethiol, and benzyl mercaptan. Non-limiting examples of a terpene-based fragrance include a linear terpene and a cyclic terpene. A linear terpene includes, without limitation, myrcene, geraniol, nerol, citral, citronellal, citronellol, linalool, and nerolidol. A cyclic terpene includes, without limitation, limonene, camphor, menthol, carvone, terpineol, α-ionone, thujone, and eucalyptol.

Other non-limiting examples of a fragrance disclosed herein include, without limitation, acetoin, ambergris, ambrein, ambroxide, artemisia pallens, balm of Gilead, balsam of Peru, bdellium, benzoin, benzyl acetate, benzyl alcohol, benzyl benzoate, benzyl cinnamate, benzylacetone, bisabolol, bourgeonal, cajuput oil, calone, camphene, camphor, caraway, cashmeran, cedar wood, chavicol, cinnamyl alcohol, citral, citronellol, civet, civetone, copaiba, cyclododecanone, cyclohexadecanone, cyclopentadecanolide, cyclopentanone, cyperus articulates, cypress oil, damascenone, damascene, deer musk, diethyl malonate, dill, estragole, ethyl benzoate, ethyl maltol, ethyl methylphenylglycidate, ethyl salicylate, eugenol, evernia prunastri, farnesene, farnesol, fennel, filbertone, galaxolide, geraniol, geranyl acetate, gourmand, grapefruit mercaptan, helichyrsum, heptyl acetate, hexyl cinnamaldehyde, hydroxycitronellal, hydroxymethylpentylcyclohexenecarboxaldehyde, hyraceum, ionone, irone, iso E Super, isobornyl cyclohexanol, isovanillin, labdanum, lavandin, lemon verena, filial, p-menthane, menthone, methyl benzoate, methyl butyrate, methyl isobutyrate, methyl pivalate, muscone, musk xylene, myrcene, nag champa, nardostachys jatamansi, nerol, nerolidol, niaouli, ocimene, opopanax, palmarosa, patchouli, patchoulol, pelargonium graveolens, perillaldehyde, petitgrain, phellandrene, phenethyl alcohol, β-pinene, polianthes tuberosa, pomarose, pulegone, raspberry ketone, rhodinol, safrole, sandalore, sandalwood, α-santalol, β-santalol, skatole, spikenard, storax balsam, synthetic musk, tagete, tetramethyl acetyloctahydronaphthalene, thujone, tincture of benzoin, tolu balsam, valeriana celtica, vanillin, ortho-vanillin and α-vetivone.

Further examples of a fragrance include, without limitation, geraniol, geranyl acetate, linalool, linalyl acetate, tetrahydrolinalool, citronellol, citronellyl acetate, dihydromyrcenol, dihydromyrcenyl acetate, tetrahydromyrcenol, terpineol, terpinyl acetate, nopol, nopyl acetate, 2-phenylethanol, 2-phenylethyl acetate, benzyl alcohol, benzyl acetate, benzyl salicylate, benzyl benzoate, styrallyl acetate, amyl salicylate, dimethylbenzyl carbinol, trichloromethylphenylcarbinyl acetate, p-tert-butyl-cyclohexyl acetate, isononyl acetate, vetiver, vetiveryl acetate, vetiverol, alpha-n-amylcinammic aldehyde, alpha-hexylcinammic aldehyde, 2-methyl-3-(p-tert-butylphenyl)-propanol, 2-methyl-3-(p-isopropylphenyl)-propanal, 3-(p-tert-butylphenyl)-propanal, tricyclodecenyl acetate, tricyclodecenyl propionate, 4-(4-hydroxy-4-methylpentyl)-3-cyclohexene carbaldehyde, 4-(4-methyl-3-pentenyl)-3-cyclohexene carbaldehyde, 4-acetoxy-3-pentyletetrahydropyran, methyl-dihydrojasmonate, 2-n-heptylcyclopentanone, 3-methyl-2-pentylcyclopentanone, n-decanal, 9-decenol-I, phenoxyethyl isobutyrate, phenylacetaldehyde dimethyl acetal, phenylacetaldehyde diethyl acetal, geranonitrile, citronellonitrile, cedryl acetate, 3-isocamphycyclohexanol, cedryl methyl ether, isolongifolanone, aubepine nitrile, aubepine, heliotropine, coumarin, eugenol, vanillin, diphenyl oxide, hydroxycitronellal, ionones, methylionones, isomethylioniones, irones, cis-3-hexenol and esters thereof, indane musk, tetralin musk, isochroman musk, macrocyclic ketones, macrolactone musk, ethylene brassylate, aromatic nitro-musk. Exemplary fragrant molecules include bergamot oil, coriander oil, dimethyl heptanol, dimethyl benzyl carbinyl acetate, geranyl acetate, citronellyl acetate, rose synthetic, geranium bourbon, hedione, iso eugenol, methyl eugenol styrallyl acetate, stemone, rose oxide laevo, aldehyde C-II undecyclic, derivatives of 2,6-dimethyl-2-alkoxy octan-7-ol, vertivert oil, vetiverol, vetiveryl, acetate, quaiac wood oil, esters of anthranilic acid, benzyl salicylate, benzyl benzoate, oak moss, eugenol, p-tert-butyl cyclohexyl acetate and coumarin.

A grip enhancing composition disclosed herein does not comprise an alcohol. An alcohol is an organic molecule comprising a hydroxyl functional group (—OH) bond to a carbon atom, where the carbon atom is saturated. In aspects of this embodiment, the alcohol not included in a grip enhancing composition disclosed herein may be, e.g., a C₂₋₄ alcohol, a C₁₋₄ alcohol, a C₁₋₅ alcohol, a C₁₋₇ alcohol, a C₁₋₁₀ alcohol, a C₁₋₁₅ alcohol, or a C₁₋₂₀ alcohol. In other aspects of this embodiment, an alcohol may be, e.g., a primary alcohol, a secondary alcohol, or a tertiary alcohol. In other aspects of this embodiment, an alcohol may be a monohydric alcohol, including, without limitation, methanol, ethanol, 1-propanol, isopropanol (2-propanol), 1-butanol, isobutanol, methyl-1-butanol, 2-butanol, tert-butanol, 1-pentanol, 2-pentanol, isoamyl alcohol, tert-amyl alcohol, 1-hexanol, 2-hexanol, 2-methyl-2-pentanol, 1-heptanol, 2-heptanol, 1-octoanol, 2-octoanol, phenethyl alcohol, 1-nonanol, 1-decanol, tryptophol, undecanol, and dodecanol, or any combination thereof.

A grip enhancing composition disclosed herein may comprise a carrier. A carrier, also known as a vehicle, can be any material typically known in the skin care, cosmetic and medical arts that is used as a base to formulate a grip enhancing composition disclosed herein. A carrier may be an aqueous carrier, a semi-solid carrier or a solid carrier. A carrier can also provide a skin care benefit as disclosed herein. A carrier includes, without limitation, water, a vegetable oil, a mineral oil, an ester oil, an ether, a fatty alcohol, an isoparaffin, a hydrocarbon oil, a polyol, and a wax. Non-limiting examples of an ester oil include octal palmitate, isopropyl myristate and isopropyl palmitate. Non-limiting examples of an ether includes dicapryl ether and dimethyl isosorbide. Non-limiting examples of a fatty alcohol include cetyl alcohol, cetearyl alcohol, stearyl alcohol and behenyl alcohol. Non-limiting examples of an isoparaffin include isooctane, isododecane (IDD) and isohexadecane. Non-limiting examples of a hydrocarbon oil include mineral oil, petrolatum, isoeicosane and a polyolefin, including (hydrogenated) polyisobutene. Non-limiting examples of a polyol include propylene glycol, glycerin, butylene glycol, pentylene glycol, hexylene glycol, caprylyl glycol. Non-limiting examples of a wax include beeswax, carnauba, ozokerite, microcrystalline wax, polyethylene wax, and a botanical wax.

In one embodiment, a single carrier is present in a grip enhancing composition disclosed herein. In another embodiment, a plurality of carriers is present in a grip enhancing composition disclosed herein. In aspects of this embodiment, a grip enhancing composition disclosed herein comprises, e.g., one or more carriers, two or more carriers, three or more carriers, four or more carriers or five or more carriers. In other aspects of this embodiment, a grip enhancing composition disclosed herein comprises, e.g., only one carrier, at most two carriers, at most three carriers, at most four carriers, or at most five carriers. In yet other aspects of this embodiment, a grip enhancing composition disclosed herein comprises from, e.g., 1 to 2 carriers, 1 to 3 carriers, 1 to 4 carriers, 1 to 5 carriers, 2 to 3 carriers, 2 to 4 carriers, 2 to 5 carriers, 3 to 4 carriers, 3 to 5 carriers or 4 to 5 carriers.

In another embodiment, a grip enhancing composition disclosed herein comprises an amount of carrier that provides a desired formulative or beneficial effect to a grip enhancing composition disclosed herein. In aspects of this embodiment, a grip enhancing composition disclosed herein comprises a carrier in an amount of, e.g., at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% or at least 99% by weight of the composition. In other aspects of this embodiment, a grip enhancing composition disclosed herein comprises a carrier in an amount of, e.g., at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 96%, at most 97%, at most 98% or at most 99% by weight of the composition. In yet other aspects of this embodiment, a grip enhancing composition disclosed herein comprises a carrier in an amount of from, e.g., about 25% to about 50%, about 25% to about 75%, about 25% to about 90%, about 25% to about 95%, about 25% to about 96%, about 25% to about 97%, about 25% to about 98%, about 25% to about 99%, about 50% to about 75%, about 50% to about 90%, about 50% to about 95%, about 50% to about 96%, about 50% to about 97%, about 50% to about 98%, about 50% to about 99%, about 75% to about 80%, about 75% to about 85%, about 75% to about 90%, about 75% to about 95%, about 75% to about 96%, about 75% to about 97%, about 75% to about 98%, about 75% to about 99%, about 80% to about 85%, about 80% to about 90%, about 80% to about 95%, about 80% to about 96%, about 80% to about 97%, about 80% to about 98%, about 80% to about 99%, about 85% to about 90%, about 85% to about 95%, about 85% to about 96%, about 85% to about 97%, about 85% to about 98%, about 85% to about 99%, about 90% to about 95%, about 90% to about 96%, about 90% to about 97%, about 90% to about 98%, about 90% to about 99%, or about 95% to about 99%, by weight of the composition.

In another embodiment, a grip enhancing composition disclosed herein comprises a buffer or buffered solution. A buffer or buffered solution can be varied as appropriate by one skilled in the art and generally depends, in part, on the pH value desired for the mobile phase, the protein being eluted, and the conductivity values being employed. Therefore, aspects of this embodiment may optionally include, e.g., 2-(N-morpholino) ethanesulfonic acid (MES), N-(2-acetamido)iminodiacetic acid (ADA), dimethylarsinic acid (Cacodylate), piperazine-N,N′-bis(2-ethanesulfonic acid) (PIPES), N-(2-Acetamido)-2-aminoethanesulfonic acid (ACES), cholamine chloride, N,N′-bis(2-hydroxyethyl)-2-aminoethanesulfonic acid (BES), 3-(N-morpholino) propanesulfonic acid (MOPS), 2-{[tris(hydroxymethyl)methyl]amino} ethanesulfonic acid (TES), N-(2-hydroxyethyl) piperazine-N′-(2-ethanesulfonic acid) (HEPES), piperazine-N,N′-bis(2-hydroxypropanesulfonic acid) (POPSO), N-tris(hydroxymethyl) methylglycine (Tricine), tris(hydroxymethyl)methylamine (Tris), acetamidoglycine, N,N-bis(2-hydroxyethyl)glycine (Bicine), N-tris(hydroxymethyl)methyl-3-aminopropanesulfonic acid (TAPS), 3-[(1,1-dimethyl-2-hydroxyethyl)amino]-2-hydroxypropanesulfonic acid (AMPSO), 3-(cyclohexylamino)-2-hydroxy-1-propanesulfonic acid (CAPSO), and 3-(cyclohexylamino)-1-propanesulfonic acid (CAPS); acetate buffers, such as, e.g., magnesium acetate, potassium acetate, and Tris acetate; borate buffers; citrate buffers; phosphate buffers, such as, e.g., potassium phosphate buffers and sodium phosphate buffers; saline buffers, such as, e.g., phosphate-buffered saline (PBS), HEPES-buffered saline (HBS), and Tris-buffered saline (TBS), saline sodium citrate (SSC); universal buffers, such as, e.g., buffers comprising citric acid and potassium phosphate, Britton-Robinson buffer, Carmody buffer and the like, or any combination thereof. Non-limiting examples of how to make and use specific buffers are described in, e.g., MOLECULAR CLONING, A LABORATORY MANUAL (Joseph Sambrook & David W. Russell eds., Cold Spring Harbor Laboratory Press, 3^{rd} ed. 2001) and CURRENT PROTOCOLS IN MOLECULAR BIOLOGY (Frederick M. Ausubel et al., eds. John Wiley & Sons, 2004).

It is understood that acids or bases can be used to adjust the pH of a buffered solution disclosed herein as needed. In an aspect of this embodiment, an effective pH level of a buffered solution disclosed herein is, e.g., at least pH 5.0, at least pH 5.5, at least pH 6.0, at least pH 6.5, at least pH 7.0, at least pH 7.5, at least pH 8.0, at least pH 8.5, at least pH 9.0, at least pH 9.5, at least pH 10.0, at least pH 10.5, at least pH 11.0, at least pH 11.5, or at least pH 12.0. In another aspect of this embodiment, an effective pH level of a buffered solution disclosed herein is, e.g., at most pH 5.0, at most pH 5.5, at most pH 6.0, at most pH 6.5, at most pH 7.0, at most pH 7.5, at most pH 8.0, at most pH 8.5, at most pH 9.0, at most pH 9.5, at most pH 10.0, at most pH 10.5, at most pH 11.0, at most pH 11.5, or at most pH 12.0. In yet another aspect of this embodiment, an effective pH level of a buffered solution disclosed herein is, e.g., about pH 5.0 to about pH 8.0, about pH 5.0 to about pH 8.5, about pH 5.0 to about pH 9.0, about pH 5.0 to about pH 9.5, about pH 5.0 to about pH 10.0, about pH 5.0 to about pH 10.5, about pH 6.0 to about pH 8.0, about pH 6.0 to about pH 8.5, about pH 6.0 to about pH 9.0, about pH 6.0 to about pH 9.5, about pH 6.0 to about pH 10.0, about pH 6.0 to about pH 10.5, about pH 7.0 to about pH 8.0, about pH 7.0 to about pH 8.5, about pH 7.0 to about pH 9.0, about pH 7.0 to about pH 9.5, about pH 7.0 to about pH 10.0, about pH 7.0 to about pH 10.5, about pH 8.0 to about pH 9.0, about pH 8.0 to about pH 9.5, about pH 8.0 to about pH 10.0, about pH 8.0 to about pH 10.5, about pH 9.0 to about pH 9.5, about pH 9.0 to about pH 10.0, about pH 9.0 to about pH 10.5, about pH 9.0 to about pH 11, about pH 9.0 to about pH 11.5, about pH 9.0 to about pH 12, about pH 9.5 to about pH 10.0, about pH 9.5 to about pH 10.5, about pH 9.5 to about pH 11, about pH 9.5 to about pH 11.5, about pH 9.5 to about pH 12, about pH 10.0 to about pH 10.5, about pH 10.0 to about pH 11, about pH 10.0 to about pH 11.5, about pH 10.0 to about pH 12, about pH 10.5 to about pH 11, about pH 10.5 to about pH 11.5, about pH 10.5 to about pH 12, about pH 11.0 to about pH 11.5, about pH 11.0 to about pH 12, or pH 11.5 to about pH 12.

A buffered solution disclosed herein may be of any concentration, with the proviso that the concentration is useful to practice the methods disclosed herein. The buffer concentration used can be varied as appropriate by one skilled in the art and generally depends, in part, on the buffering capacity of a particular buffer being used and the other components of a grip enhancing composition disclosed herein. In aspects of this embodiment, the concentration of a buffered solution is, e.g., at least 0.1 mM, at least 0.2 mM, at least 0.3 mM, at least 0.4 mM, at least 0.5 mM, at least 0.6 mM, at least 0.7 mM, at least 0.8 mM, or at least 0.9 mM. In other aspects of this embodiment, the buffer concentration of a buffered solution is, e.g., at least 1.0 mM, at least 2.0 mM, at least 3.0 mM, at least 4.0 mM, at least 5.0 mM, at least 6.0 mM, at least 7.0 mM, at least 8.0 mM, or at least 9.0 mM. In yet other aspects of this embodiment, the concentration of a buffered solution is, e.g., at least 10 mM, at least 20 mM, at least 30 mM, at least 40 mM, at least 50 mM, at least 60 mM, at least 70 mM, at least 80 mM, or at least 90 mM. In still other aspects of this embodiment, the concentration of a buffered solution is, e.g., at least 100 mM, at least 200 mM, at least 300 mM, at least 400 mM, at least 500 mM, at least 600 mM, at least 700 mM, at least 800 mM, or at least 900 mM.

In other aspects of this embodiment, the concentration of a buffered solution is, e.g., at most 0.1 mM, at most 0.2 mM, at most 0.3 mM, at most 0.4 mM, at most 0.5 mM, at most 0.6 mM, at most 0.7 mM, at most 0.8 mM, or at most 0.9 mM. In yet other aspects of this embodiment, the concentration of a buffered solution is, e.g., at most 1.0 mM, at most 2.0 mM, at most 3.0 mM, at most 4.0 mM, at most 5.0 mM, at most 6.0 mM, at most 7.0 mM, at most 8.0 mM, or at most 9.0 mM. In still other aspects of this embodiment, the concentration of a buffered solution is, e.g., at most 10 mM, at most 20 mM, at most 30 mM, at most 40 mM, at most 50 mM, at most 60 mM, at most 70 mM, at most 80 mM, or at most 90 mM. In further aspects of this embodiment, the concentration of a buffered solution is, e.g., at most 100 mM, at most 200 mM, at most 300 mM, at most 400 mM, at most 500 mM, at most 600 mM, at most 700 mM, at most 800 mM, or at most 900 mM.

In other aspects of this embodiment, the concentration of a buffered solution is between, e.g., about 0.1 mM to about 900 mM, 0.1 mM to about 500 mM, 0.1 mM to about 100 mM, 0.1 mM to about 90 mM, 0.1 mM to about 50 mM, 1.0 mM to about 900 mM, 1.0 mM to about 500 mM, 1.0 mM to about 100 mM, 1.0 mM to about 90 mM, or 1.0 mM to about 50 mM.

A grip enhancing composition disclosed herein is stable. In one embodiment, a grip enhancing composition disclosed herein is stable when it shows, e.g., at most 1% degradation, at most 2% degradation, at most 3% degradation, at most 4% degradation, at most 5% degradation, at most 6% degradation, at most 7% degradation, at most 8% degradation, at most 9% degradation, or at most 10% degradation of hypochlorous acid. In another embodiment, a grip enhancing composition disclosed herein is stable when it shows, e.g., at most 10% degradation, at most 15% degradation, at most 20% degradation, at most 25% degradation, at most 30% degradation, at most 35% degradation, at most 40% degradation, at most 45% degradation, at most 50% degradation, at most 55% degradation, at most 60% degradation, at most 65% degradation, at most 70% degradation, at most 75% degradation, at most 80% degradation, at most 85% degradation, at most 90% degradation, or at most 95% degradation of hypochlorous acid. In another embodiment, a grip enhancing composition disclosed herein is stable when it shows, e.g., about 1% to about 10% degradation, about 1% to about 20% degradation, about 1% to about 30% degradation, about 1% to about 40% degradation, about 1% to about 50% degradation, about 1% to about 60% degradation, about 1% to about 70% degradation, about 1% to about 80% degradation, about 1% to about 90% degradation, about 1% to about 95% degradation, about 5% to about 10% degradation, about 5% to about 20% degradation, about 5% to about 30% degradation, about 5% to about 40% degradation, about 5% to about 50% degradation, about 5% to about 60% degradation, about 5% to about 70% degradation, about 5% to about 80% degradation, about 5% to about 90% degradation, about 5% to about 95% degradation, about 10% to about 20% degradation, about 10% to about 30% degradation, about 10% to about 40% degradation, about 10% to about 50% degradation, about 10% to about 60% degradation, about 10% to about 70% degradation, about 10% to about 80% degradation, about 10% to about 90% degradation, about 10% to about 95% degradation, 20% to about 30% degradation, about 20% to about 40% degradation, about 20% to about 50% degradation, about 20% to about 60% degradation, about 20% to about 70% degradation, about 20% to about 80% degradation, about 20% to about 90% degradation, about 20% to about 95% degradation, about 30% to about 40% degradation, about 30% to about 50% degradation, about 30% to about 60% degradation, about 30% to about 70% degradation, about 30% to about 80% degradation, about 30% to about 90% degradation, about 30% to about 95% degradation, about 40% to about 50% degradation, about 40% to about 60% degradation, about 40% to about 70% degradation, about 40% to about 80% degradation, about 40% to about 90% degradation, about 40% to about 95% degradation, about 50% to about 60% degradation, about 50% to about 70% degradation, about 50% to about 80% degradation, about 50% to about 90% degradation, about 50% to about 95% degradation, about 60% to about 70% degradation, about 60% to about 80% degradation, about 60% to about 90% degradation, about 60% to about 95% degradation, about 70% to about 80% degradation, about 70% to about 90% degradation, about 70% to about 95% degradation, about 80% to about 90% degradation, about 80% to about 95% degradation, or about 90% to about 95% degradation of hypochlorous acid.

In one embodiment, a grip enhancing composition disclosed herein is stable when it shows, e.g., at most 1% degradation, at most 2% degradation, at most 3% degradation, at most 4% degradation, at most 5% degradation, at most 6% degradation, at most 7% degradation, at most 8% degradation, at most 9% degradation, or at most 10% degradation of free available chlorine. In another embodiment, a grip enhancing composition disclosed herein is stable when it shows, e.g., at most 10% degradation, at most 15% degradation, at most 20% degradation, at most 25% degradation, at most 30% degradation, at most 35% degradation, at most 40% degradation, at most 45% degradation, at most 50% degradation, at most 55% degradation, at most 60% degradation, at most 65% degradation, at most 70% degradation, at most 75% degradation, at most 80% degradation, at most 85% degradation, at most 90% degradation, or at most 95% degradation of free available chlorine. In another embodiment, a grip enhancing composition disclosed herein is stable when it shows, e.g., about 1% to about 10% degradation, about 1% to about 20% degradation, about 1% to about 30% degradation, about 1% to about 40% degradation, about 1% to about 50% degradation, about 1% to about 60% degradation, about 1% to about 70% degradation, about 1% to about 80% degradation, about 1% to about 90% degradation, about 1% to about 95% degradation, about 5% to about 10% degradation, about 5% to about 20% degradation, about 5% to about 30% degradation, about 5% to about 40% degradation, about 5% to about 50% degradation, about 5% to about 60% degradation, about 5% to about 70% degradation, about 5% to about 80% degradation, about 5% to about 90% degradation, about 5% to about 95% degradation, about 10% to about 20% degradation, about 10% to about 30% degradation, about 10% to about 40% degradation, about 10% to about 50% degradation, about 10% to about 60% degradation, about 10% to about 70% degradation, about 10% to about 80% degradation, about 10% to about 90% degradation, about 10% to about 95% degradation, 20% to about 30% degradation, about 20% to about 40% degradation, about 20% to about 50% degradation, about 20% to about 60% degradation, about 20% to about 70% degradation, about 20% to about 80% degradation, about 20% to about 90% degradation, about 20% to about 95% degradation, about 30% to about 40% degradation, about 30% to about 50% degradation, about 30% to about 60% degradation, about 30% to about 70% degradation, about 30% to about 80% degradation, about 30% to about 90% degradation, about 30% to about 95% degradation, about 40% to about 50% degradation, about 40% to about 60% degradation, about 40% to about 70% degradation, about 40% to about 80% degradation, about 40% to about 90% degradation, about 40% to about 95% degradation, about 50% to about 60% degradation, about 50% to about 70% degradation, about 50% to about 80% degradation, about 50% to about 90% degradation, about 50% to about 95% degradation, about 60% to about 70% degradation, about 60% to about 80% degradation, about 60% to about 90% degradation, about 60% to about 95% degradation, about 70% to about 80% degradation, about 70% to about 90% degradation, about 70% to about 95% degradation, about 80% to about 90% degradation, about 80% to about 95% degradation, or about 90% to about 95% degradation of free available chlorine.

In aspects of this embodiment, a grip enhancing composition disclosed herein is stable for, e.g., about 1 day, about 2 days, 3 days, 4 days, 5 days, 6, days, 7 days, 8 days, 9 days, 10 days, 11 days, 12 days, 13 days, 14 days or 15 days. In other aspects of this embodiment, a grip enhancing composition disclosed herein is stable for, e.g., at least 1 day, at least 2 days, at least 3 days, at least 4 days, at least 5 days, at least 6, days, at least 7 days, at least 8 days, at least 9 days, at least 10 days, at least 11 days, at least 12 days, at least 13 days, at least 14 days or at least 15 days. In yet other aspects of this embodiment, a grip enhancing composition disclosed herein is stable for, e.g., at most 1 day, at most 2 days, at most 3 days, at most 4 days, at most 5 days, at most 6, days, at most 7 days, at most 8 days, at most 9 days, at most 10 days, at most 11 days, at most 12 days, at most 13 days, at most 14 days or at most 15 days.

In still aspects of this embodiment, a grip enhancing composition disclosed herein is stable for, e.g., about 1 day to about 2 days, about 1 day to about 3 days, about 1 day to about 4 days, about 1 day to about 5 days, about 1 day to about 6 days, about 1 day to about 7 days, about 1 day to about 8 days, about 1 day to about 9 days, about 1 day to about 10 days, about 1 day to about 11 days, about 1 day to about 12 days, about 1 day to about 13 days, about 1 day to about 14 days, about 1 day to about 15 days, about 2 days to about 3 days, about 2 days to about 4 days, about 2 days to about 5 days, about 2 days to about 6 days, about 2 days to about 7 days, about 2 days to about 8 days, about 2 days to about 9 days, about 2 days to about 10 days, about 2 days to about 11 days, about 2 days to about 12 days, about 2 days to about 13 days, about 2 days to about 14 days, about 2 days to about 15 days, about 3 days to about 4 days, about 3 days to about 5 days, about 3 days to about 6 days, about 3 days to about 7 days, about 3 days to about 8 days, about 3 days to about 9 days, about 3 days to about 10 days, about 3 days to about 11 days, about 3 days to about 12 days, about 3 days to about 13 days, about 3 days to about 14 days, about 3 days to about 15 days, about 4 days to about 5 days, about 4 days to about 6 days, about 4 days to about 7 days, about 4 days to about 8 days, about 4 days to about 9 days, about 4 days to about 10 days, about 4 days to about 11 days, about 4 days to about 12 days, about 4 days to about 13 days, about 4 days to about 14 days, about 4 days to about 15 days, about 5 days to about 6 days, about 5 days to about 7 days, about 5 days to about 8 days, about 5 days to about 9 days, about 5 days to about 10 days, about 5 days to about 11 days, about 5 days to about 12 days, about 5 days to about 13 days, about 5 days to about 14 days, about 5 days to about 15 days, about 6 days to about 7 days, about 6 days to about 8 days, about 6 days to about 9 days, about 6 days to about 10 days, about 6 days to about 11 days, about 6 days to about 12 days, about 6 days to about 13 days, about 6 days to about 14 days, about 6 days to about 15 days, about 7 days to about 8 days, about 7 days to about 9 days, about 7 days to about 10 days, about 7 days to about 11 days, about 7 days to about 12 days, about 7 days to about 13 days, about 7 days to about 14 days, about 7 days to about 15 days, about 8 days to about 9 days, about 8 days to about 10 days, about 8 days to about 11 days, about 8 days to about 12 days, about 8 days to about 13 days, about 8 days to about 14 days, about 8 days to about 15 days, about 9 days to about 10 days, about 9 days to about 11 days, about 9 days to about 12 days, about 9 days to about 13 days, about 9 days to about 14 days, about 9 days to about 15 days, about 10 days to about 11 days, about 10 days to about 12 days, about 10 days to about 13 days, about 10 days to about 14 days, about 10 days to about 15 days, about 11 days to about 12 days, about 11 days to about 13 days, about 11 days to about 14 days, about 11 days to about 15 days, about 12 days to about 13 days, about 12 days to about 14 days, about 12 days to about 15 days, about 13 days to about 14 days, about 13 days to about 15 days, or about 14 days to about 15 days.

In other aspects of this embodiment, a grip enhancing composition disclosed herein is stable for, e.g., about 1 week, about 2 weeks, 3 weeks, 4 weeks, 5 weeks, 6, weeks, 7 weeks, 8 weeks, 9 weeks, 10 weeks, 11 weeks, 12 weeks, 13 weeks, 14 weeks or 15 weeks. In yet other aspects of this embodiment, a grip enhancing composition disclosed herein is stable for, e.g., at least 1 week, at least 2 weeks, at least 3 weeks, at least 4 weeks, at least 5 weeks, at least 6, weeks, at least 7 weeks, at least 8 weeks, at least 9 weeks, at least 10 weeks, at least 11 weeks, at least 12 weeks, at least 13 weeks, at least 14 weeks or at least 15 weeks. In still other aspects of this embodiment, a grip enhancing composition disclosed herein is stable for, e.g., at most 1 week, at most 2 weeks, at most 3 weeks, at most 4 weeks, at most 5 weeks, at most 6, weeks, at most 7 weeks, at most 8 weeks, at most 9 weeks, at most 10 weeks, at most 11 weeks, at most 12 weeks, at most 13 weeks, at most 14 weeks or at most 15 weeks.

In aspects of this embodiment, a grip enhancing composition disclosed herein is stable for, e.g., about 1 week to about 2 weeks, about 1 week to about 3 weeks, about 1 week to about 4 weeks, about 1 week to about 5 weeks, about 1 week to about 6 weeks, about 1 week to about 7 weeks, about 1 week to about 8 weeks, about 1 week to about 9 weeks, about 1 week to about 10 weeks, about 1 week to about 11 weeks, about 1 week to about 12 weeks, about 1 week to about 13 weeks, about 1 week to about 14 weeks, about 1 week to about 15 weeks, about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 2 weeks to about 11 weeks, about 2 weeks to about 12 weeks, about 2 weeks to about 13 weeks, about 2 weeks to about 14 weeks, about 2 weeks to about 15 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 3 weeks to about 11 weeks, about 3 weeks to about 12 weeks, about 3 weeks to about 13 weeks, about 3 weeks to about 14 weeks, about 3 weeks to about 15 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 4 weeks to about 11 weeks, about 4 weeks to about 12 weeks, about 4 weeks to about 13 weeks, about 4 weeks to about 14 weeks, about 4 weeks to about 15 weeks, about 5 weeks to about 6 weeks, about 5 weeks to about 7 weeks, about 5 weeks to about 8 weeks, about 5 weeks to about 9 weeks, about 5 weeks to about 10 weeks, about 5 weeks to about 11 weeks, about 5 weeks to about 12 weeks, about 5 weeks to about 13 weeks, about 5 weeks to about 14 weeks, about 5 weeks to about 15 weeks, about 6 weeks to about 7 weeks, about 6 weeks to about 8 weeks, about 6 weeks to about 9 weeks, about 6 weeks to about 10 weeks, about 6 weeks to about 11 weeks, about 6 weeks to about 12 weeks, about 6 weeks to about 13 weeks, about 6 weeks to about 14 weeks, about 6 weeks to about 15 weeks, about 7 weeks to about 8 weeks, about 7 weeks to about 9 weeks, about 7 weeks to about 10 weeks, about 7 weeks to about 11 weeks, about 7 weeks to about 12 weeks, about 7 weeks to about 13 weeks, about 7 weeks to about 14 weeks, about 7 weeks to about 15 weeks, about 8 weeks to about 9 weeks, about 8 weeks to about 10 weeks, about 8 weeks to about 11 weeks, about 8 weeks to about 12 weeks, about 8 weeks to about 13 weeks, about 8 weeks to about 14 weeks, about 8 weeks to about 15 weeks, about 9 weeks to about 10 weeks, about 9 weeks to about 11 weeks, about 9 weeks to about 12 weeks, about 9 weeks to about 13 weeks, about 9 weeks to about 14 weeks, about 9 weeks to about 15 weeks, about 10 weeks to about 11 weeks, about 10 weeks to about 12 weeks, about 10 weeks to about 13 weeks, about 10 weeks to about 14 weeks, about 10 weeks to about 15 weeks, about 11 weeks to about 12 weeks, about 11 weeks to about 13 weeks, about 11 weeks to about 14 weeks, about 11 weeks to about 15 weeks, about 12 weeks to about 13 weeks, about 12 weeks to about 14 weeks, about 12 weeks to about 15 weeks, about 13 weeks to about 14 weeks, about 13 weeks to about 15 weeks, or about 14 weeks to about 15 weeks.

In other aspects of this embodiment, a grip enhancing composition disclosed herein is stable for, e.g., about 1 month, about 2 months, 3 months, 4 months, 5 months, 6, months, 7 months, 8 months, 9 months, 10 months, 11 months, 12 months, 13 months, 14 months, 15 months, 16 months, 17 months, or 18 months. In yet other aspects of this embodiment, a grip enhancing composition disclosed herein is stable for, e.g., at least 1 month, at least 2 months, at least 3 months, at least 4 months, at least 5 months, at least 6, months, at least 7 months, at least 8 months, at least 9 months, at least 10 months, at least 11 months, at least 12 months, at least 13 months, at least 14 months, at least 15 months, at least 16 months, at least 17 months, or at least 18 months. In still other aspects of this embodiment, a grip enhancing composition disclosed herein is stable for, e.g., at most 1 month, at most 2 months, at most 3 months, at most 4 months, at most 5 months, at most 6, months, at most 7 months, at most 8 months, at most 9 months, at most 10 months, at most 11 months, at most 12 months, at most 13 months, at most 14 months, at most 15 months, at le most ast 16 months, at most 17 months, or at most 18 months.

In aspects of this embodiment, a grip enhancing composition disclosed herein is stable for, e.g., about 1 month to about 2 months, about 1 month to about 3 months, about 1 month to about 4 months, about 1 month to about 5 months, about 1 month to about 6 months, about 1 month to about 7 months, about 1 month to about 8 months, about 1 month to about 9 months, about 1 month to about 10 months, about 1 month to about 11 months, about 1 month to about 12 months, about 1 month to about 13 months, about 1 month to about 14 months, about 1 month to about 15 months, about 1 month to about 16 months, about 1 month to about 17 months, about 1 month to about 18 months, about 2 months to about 3 months, about 2 months to about 4 months, about 2 months to about 5 months, about 2 months to about 6 months, about 2 months to about 7 months, about 2 months to about 8 months, about 2 months to about 9 months, about 2 months to about 10 months, about 2 months to about 11 months, about 2 months to about 12 months, about 2 months to about 13 months, about 2 months to about 14 months, about 2 months to about 15 months, about 2 months to about 16 months, about 2 months to about 17 months, about 2 months to about 18 months, about 3 months to about 4 months, about 3 months to about 5 months, about 3 months to about 6 months, about 3 months to about 7 months, about 3 months to about 8 months, about 3 months to about 9 months, about 3 months to about 10 months, about 3 months to about 11 months, about 3 months to about 12 months, about 3 months to about 13 months, about 3 months to about 14 months, about 3 months to about 15 months, about 3 months to about 16 months, about 3 months to about 17 months, about 3 months to about 18 months, about 4 months to about 5 months, about 4 months to about 6 months, about 4 months to about 7 months, about 4 months to about 8 months, about 4 months to about 9 months, about 4 months to about 10 months, about 4 months to about 11 months, about 4 months to about 12 months, about 4 months to about 13 months, about 4 months to about 14 months, about 4 months to about 15 months, about 4 months to about 16 months, about 4 months to about 17 months, about 4 months to about 18 months, about 5 months to about 6 months, about 5 months to about 7 months, about 5 months to about 8 months, about 5 months to about 9 months, about 5 months to about 10 months, about 5 months to about 11 months, about 5 months to about 12 months, about 5 months to about 13 months, about 5 months to about 14 months, about 5 months to about 15 months, about 5 months to about 16 months, about 5 months to about 17 months, about 5 months to about 18 months, about 6 months to about 7 months, about 6 months to about 8 months, about 6 months to about 9 months, about 6 months to about 10 months, about 6 months to about 11 months, about 6 months to about 12 months, about 6 months to about 13 months, about 6 months to about 14 months, about 6 months to about 15 months, about 6 months to about 16 months, about 6 months to about 17 months, about 6 months to about 18 months, about 7 months to about 8 months, about 7 months to about 9 months, about 7 months to about 10 months, about 7 months to about 11 months, about 7 months to about 12 months, about 7 months to about 13 months, about 7 months to about 14 months, about 7 months to about 15 months, about 7 months to about 16 months, about 7 months to about 17 months, about 7 months to about 18 months, about 8 months to about 9 months, about 8 months to about 10 months, about 8 months to about 11 months, about 8 months to about 12 months, about 8 months to about 13 months, about 8 months to about 14 months, about 8 months to about 15 months, about 8 months to about 16 months, about 8 months to about 17 months, about 8 months to about 18 months, about 9 months to about 10 months, about 9 months to about 11 months, about 9 months to about 12 months, about 9 months to about 13 months, about 9 months to about 14 months, about 9 months to about 15 months, about 9 months to about 16 months, about 9 months to about 17 months, about 9 months to about 18 months, about 10 months to about 11 months, about 10 months to about 12 months, about 10 months to about 13 months, about 10 months to about 14 months, about 10 months to about 15 months, about 10 months to about 16 months, about 10 months to about 17 months, about 10 months to about 18 months, about 11 months to about 12 months, about 11 months to about 13 months, about 11 months to about 14 months, about 11 months to about 15 months, about 11 months to about 16 months, about 11 months to about 17 months, about 1 months to about 18 months, about 12 months to about 13 months, about 12 months to about 14 months, about 12 months to about 15 months, about 12 months to about 16 months, about 12 months to about 17 months, about 12 months to about 18 months, about 13 months to about 14 months, about 13 months to about 15 months, about 13 months to about 16 months, about 13 months to about 17 months, about 13 months to about 18 months, about 14 months to about 15 months, about 14 months to about 16 months, about 14 months to about 17 months, about 14 months to about 18 months, about 15 months to about 16 months, about 15 months to about 17 months, about 15 months to about 18 months, about 16 months to about 17 months, about 16 months to about 18 months, or about 17 months to about 18 months.

In other aspects of this embodiment, a grip enhancing composition disclosed herein is stable for, e.g., about 1 year, about 2 years, about 3 years, about 4 years, or about 5 years. In yet other aspects of this embodiment, a grip enhancing composition disclosed herein is stable for, e.g., at least 1 year, at least 2 years, at least 3 years, at least 4 years, or at least 5 years. In still other aspects of this embodiment, a grip enhancing composition disclosed herein is stable for, e.g., at most 1 year, at most 2 years, at most 3 years, at most 4 years, or at most 5 years. In other aspects of this embodiment, a grip enhancing composition disclosed herein is stable for, e.g., about 1 year to about 2 years, about 1 year to about 3 years, about 1 year to about 4 years, about 1 year to about 5 years, about 2 years to about 3 years, about 2 years to about 4 years, about 2 years to about 5 years, about 3 years to about 4 years, about 3 years to about 5 years, or about 4 years to about 5 years.

A grip enhancing composition disclosed herein can be formulated into any form that enables topical application of the composition disclosed herein in a manner that achieves a desired beneficial effect. In one embodiment, a grip enhancing composition disclosed herein can be formulated into, e.g., a single-phase formulation, a biphasic formulation comprising a medium phase and a dispersed phase, or a multiphasic formulation. In another embodiment, a grip enhancing composition disclosed herein can be formulated into, e.g., a liquid composition, a colloidal composition, a semi-solid composition, or a solid composition. In another embodiment, a grip enhancing composition disclosed herein can be formulated into, e.g., a spray, a liquid aerosol, a wash, a lotion, a cream, a foam, an emulsion, a suspension, a gel, a sol, a wet powder, a pressed powder, or a solid sol.

A grip enhancing composition disclosed herein can be formulated to have a viscosity. The degree of viscosity of a grip enhancing composition disclosed herein will typically depend on the topical application of the composition. In some uses, a grip enhancing composition disclosed herein will have a lower viscosity while in others the composition will have a higher viscosity. For example, a spray will have a low viscosity while a gel will have a higher viscosity. The desired viscosity of a grip enhancing composition disclosed herein can be achieved by the type and amount of hydrophilic clays used, the type and amount of polysaccharides used, as well as by the final degree of moisture content present in a grip enhancing composition disclosed herein.

In an embodiment, a grip enhancing composition can be manufactured in a manner that reduces moisture content. In aspects of this embodiment, a manufacturing process may contain as an optional step a treatment that reduces the moisture content of a grip enhancing composition disclosed herein. For example, a grip enhancing composition disclosed herein can be treated by heating at a certain temperature for a specified amount of time in order to evaporate moisture from the grip composition. In aspects of this embodiment, a grip enhancing composition disclosed herein is heated to a temperature of about 150° C. to about 250° C. for about 10 minutes to 60 minutes.

In aspects of this embodiment, a grip enhancing composition disclosed herein is heated to a temperature of about 150° C. to about 250° C. for about 10 minutes to 60 minutes to obtain a final moisture content that can be, e.g., at least 10% less by weight, at least 20% less by weight, at least 30% less by weight, at least 40% less by weight, at least 50% less by weight, at least 60% less by weight, at least 70% less by weight, or at least 80% less by weight, of the moisture content of a grip enhancing composition disclosed herein without such treated. In other aspects of this embodiment, a grip enhancing composition disclosed herein is heated to a temperature of about 150° C. to about 250° C. for about 10 minutes to 60 minutes to obtain a final moisture content that can be, e.g., at most 10% less by weight, at most 20% less by weight, at most 30% less by weight, at most 40% less by weight, at most 50% less by weight, at most 60% less by weight, at most 70% less by weight, or at most 80% less by weight, of the moisture content of a grip enhancing composition disclosed herein without such treated. In yet other aspects of this embodiment, a grip enhancing composition disclosed herein is heated to a temperature of about 150° C. to about 250° C. for about 10 minutes to 60 minutes to obtain a final moisture content that can be, e.g., about 10% to about 20%, about 10% to about 30%, about 10% to about 40%, about 10% to about 50%, about 10% to about 60%, about 10% to about 70%, about 10% to about 80%, about 20% to about 30%, about 20% to about 40%, about 20% to about 50%, about 20% to about 60%, about 20% to about 70%, about 20% to about 80%, about 30% to about 40%, about 30% to about 50%, about 30% to about 60%, about 30% to about 70%, about 30% to about 80%, about 40% to about 50%, about 40% to about 60%, about 40% to about 70%, about 40% to about 80%, about 50% to about 60%, about 50% to about 70%, about 50% to about 80%, about 60% to about 70%, about 60% to about 80%, or about 70% to about 80%, less by weight than that of the moisture content of a grip enhancing composition disclosed herein without such treated.

In aspects of this embodiment, a grip enhancing composition disclosed herein has a viscosity of, e.g., about 100 mPa·s, about 250 mPa·s, about 500 mPa·s, about 750 mPa·s, about 1,000 mPa·s, about 1,250 mPa·s, about 1,500 mPa·s, about 1,750 mPa·s, about 2,000 mPa·s, about 2,250 mPa·s, about 2,500 mPa·s, about 2,750 mPa·s, about 3,000 mPa·s, about 3,250 mPa·s, about 3,500 mPa·s, about 3,750 mPa·s, about 4,000 mPa·s, about 4,250 mPa·s, about 4,500 mPa·s, about 4,750 mPa·s, about 5,000 mPa·s, about 5,250 mPa·s, about 5,500 mPa·s, about 5,750 mPa·s, about 6,000 mPa·s, about 6,250 mPa·s, about 6,500 mPa·s, about 6,750 mPa·s, about 7,000 mPa·s, about 7,250 mPa·s, about 7,500 mPa·s, about 7,750 mPa·s, about 8,000 mPa·s, about 8,250 mPa·s, about 8,500 mPa·s, about 8,750 mPa·s, about 9,000 mPa·s, about 9,250 mPa·s, about 9,500 mPa·s, about 9,750 mPa·s, about 10,000 mPa·s, about 10,250 mPa·s, about 10,500 mPa·s, about 10,750 mPa·s, about 11,000 mPa·s, about 11,250 mPa·s, about 11,500 mPa·s, about 11,750 mPa·s, about 12,000 mPa·s, about 12,250 mPa·s, about 12,500 mPa·s, about 12,750 mPa·s, about 13,000 mPa·s, about 13,250 mPa·s, about 13,500 mPa·s, about 13,750 mPa·s, about 14,000 mPa·s, about 14,250 mPa·s, about 14,500 mPa·s, about 14,750 mPa·s, about 15,000 mPa·s, about 15,500 mPa·s, about 16,000 mPa·s, about 16,500 mPa·s, about 17,000 mPa·s, about 17,500 mPa·s, about 18,000 mPa·s, about 18,500 mPa·s, about 19,000 mPa·s, about 19,500 mPa·s, or about 20,000 mPa·s. In other aspects of this embodiment, a grip enhancing composition disclosed herein has a viscosity of, e.g., at least 100 mPa·s, at least 500 mPa·s, at least 1,000 mPa·s, at least 2,000 mPa·s, at least 3,000 mPa·s, at least 4,000 mPa·s, at least 5,000 mPa·s, at least 6,000 mPa·s, at least 7,000 mPa·s, at least 8,000 mPa·s, at least 9,000 mPa·s, at least 10,000 mPa·s, at least 11,000 mPa·s, at least 12,000 mPa·s, at least 13,000 mPa·s, at least 14,000 mPa·s, at least 15,000 mPa·s, at least 16,000 mPa·s, at least 17,000 mPa·s, at least 18,000 mPa·s, at least 19,000 mPa·s, or at least 20,000 mPa·s. In yet other aspects of this embodiment, a grip enhancing composition disclosed herein has a viscosity of, e.g., at most 100 mPa·s, at most 500 mPa·s, at most 1,000 mPa·s, at most 2,000 mPa·s, at most 3,000 mPa·s, at most 4,000 mPa·s, at most 5,000 mPa·s, at most 6,000 mPa·s, at most 7,000 mPa·s, at most 8,000 mPa·s, at most 9,000 mPa·s, at most 10,000 mPa·s, at most 11,000 mPa·s, at most 12,000 mPa·s, at most 13,000 mPa·s, at most 14,000 mPa·s, at most 15,000 mPa·s, at most 16,000 mPa·s, at most 17,000 mPa·s, at most 18,000 mPa·s, at most 19,000 mPa·s, or at most 20,000 mPa·s.

In still other aspects of this embodiment, a grip enhancing composition disclosed herein has a viscosity of, e.g., about 100 mPa·s to about 500 mPa·s, about 100 mPa·s to about 1,000 mPa·s, about 100 mPa·s to about 2,000 mPa·s, about 100 mPa·s to about 3,000 mPa·s, about 100 mPa·s to about 4,000 mPa·s, about 100 mPa·s to about 5,000 mPa·s, about 100 mPa·s to about 6,000 mPa·s, about 100 mPa·s to about 7,000 mPa·s, about 100 mPa·s to about 8,000 mPa·s, about 100 mPa·s to about 9,000 mPa·s, about 100 mPa·s to about 10,000 mPa·s, about 100 mPa·s to about 11,000 mPa·s, about 100 mPa·s to about 12,000 mPa·s, about 100 mPa·s to about 13,000 mPa·s, about 100 mPa·s to about 14,000 mPa·s, about 100 mPa·s to about 15,000 mPa·s, about 100 mPa·s to about 16,000 mPa·s, about 100 mPa·s to about 17,000 mPa·s, about 100 mPa·s to about 18,000 mPa·s, about 100 mPa·s to about 19,000 mPa·s, or about 100 mPa·s to about 20,000 mPa·s, about 1,000 mPa·s to about 2,000 mPa·s, about 1,000 mPa·s to about 3,000 mPa·s, about 1,000 mPa·s to about 4,000 mPa·s, about 1,000 mPa·s to about 5,000 mPa·s, about 1,000 mPa·s to about 6,000 mPa·s, about 1,000 mPa·s to about 7,000 mPa·s, about 1,000 mPa·s to about 8,000 mPa·s, about 1,000 mPa·s to about 9,000 mPa·s, about 1,000 mPa·s to about 10,000 mPa·s, about 1,000 mPa·s to about 11,000 mPa·s, about 1,000 mPa·s to about 12,000 mPa·s, about 1,000 mPa·s to about 13,000 mPa·s, about 1,000 mPa·s to about 14,000 mPa·s, about 1,000 mPa·s to about 15,000 mPa·s, about 1,000 mPa·s to about 16,000 mPa·s, about 1,000 mPa·s to about 17,000 mPa·s, about 1,000 mPa·s to about 18,000 mPa·s, about 1,000 mPa·s to about 19,000 mPa·s, or about 1,000 mPa·s to about 20,000 mPa·s, about 2,000 mPa·s to about 3,000 mPa·s, about 2,000 mPa·s to about 4,000 mPa·s, about 2,000 mPa·s to about 5,000 mPa·s, about 2,000 mPa·s to about 6,000 mPa·s, about 2,000 mPa·s to about 7,000 mPa·s, about 2,000 mPa·s to about 8,000 mPa·s, about 2,000 mPa·s to about 9,000 mPa·s, about 2,000 mPa·s to about 10,000 mPa·s, about 2,000 mPa·s to about 11,000 mPa·s, about 2,000 mPa·s to about 12,000 mPa·s, about 2,000 mPa·s to about 13,000 mPa·s, about 2,000 mPa·s to about 14,000 mPa·s, about 2,000 mPa·s to about 15,000 mPa·s, about 2,000 mPa·s to about 16,000 mPa·s, about 2,000 mPa·s to about 17,000 mPa·s, about 2,000 mPa·s to about 18,000 mPa·s, about 2,000 mPa·s to about 19,000 mPa·s, or about 2,000 mPa·s to about 20,000 mPa·s, about 2,500 mPa·s to about 5,000 mPa·s, about 2,500 mPa·s to about 7,500 mPa·s, about 2,500 mPa·s to about 10,000 mPa·s, about 2,500 mPa·s to about 12,500 mPa·s, about 2,500 mPa·s to about 15,000 mPa·s, about 2,500 mPa·s to about 17,500 mPa·s, about 2,500 mPa·s to about 20,000 mPa·s, about 5,000 mPa·s to about 7,500 mPa·s, about 5,000 mPa·s to about 10,000 mPa·s, about 5,000 mPa·s to about 12,500 mPa·s, about 5,000 mPa·s to about 15,000 mPa·s, about 5,000 mPa·s to about 17,500 mPa·s, about 5,000 mPa·s to about 20,000 mPa·s, about 7,500 mPa·s to about 10,000 mPa·s, about 7,500 mPa·s to about 12,500 mPa·s, about 7,500 mPa·s to about 15,000 mPa·s, about 7,500 mPa·s to about 17,500 mPa·s, about 7,500 mPa·s to about 20,000 mPa·s, about 10,000 mPa·s to about 12,500 mPa·s, about 10,000 mPa·s to about 15,000 mPa·s, about 10,000 mPa·s to about 17,500 mPa·s, about 10,000 mPa·s to about 20,000 mPa·s, about 12,500 mPa·s to about 15,000 mPa·s, about 12,500 mPa·s to about 17,500 mPa·s, about 12,500 mPa·s to about 20,000 mPa·s, about 15,000 mPa·s to about 17,500 mPa·s, about 15,000 mPa·s to about 20,000 mPa·s, or about 17,500 mPa·s to about 20,000 mPa·s.

In one embodiment, a grip enhancing composition disclosed herein comprises one or more grip enhancers and one or more hydrophilic clays. In aspects of this embodiment, a grip enhancing composition disclosed herein comprises about 18% to about 28% of one or more grip enhancers and about 1.0% to about 2.5% one or more hydrophilic clays. In other aspects of this embodiment, a grip enhancing composition disclosed herein comprises about 20% to about 26% of one or more grip enhancers and about 1.5% to about 2.1% one or more hydrophilic clays. In yet other aspects of this embodiment, a grip enhancing composition disclosed herein comprises about 21% to about 25% of one or more grip enhancers and about 1.6% to about 2.0% one or more hydrophilic clays. In still other aspects of this embodiment, a grip enhancing composition disclosed herein comprises about 22% to about 24% of one or more grip enhancers and about 1.7% to about 1.9% one or more hydrophilic clays. In other aspects of this embodiment, a grip enhancing composition disclosed herein comprises about 23% of one or more grip enhancers and about 1.8% one or more hydrophilic clays. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, hypochlorous acid or free available chlorine, or any combination thereof.

In aspects of this embodiment, a grip enhancing composition disclosed herein comprises about 18% to about 28% of one or more chalks and about 1.0% to about 2.5% one or more silicate clays. In other aspects of this embodiment, a grip enhancing composition disclosed herein comprises about 20% to about 26% of one or more chalks and about 1.5% to about 2.1% one or more silicate clays. In yet other aspects of this embodiment, a grip enhancing composition disclosed herein comprises about 21% to about 25% of one or more chalks and about 1.6% to about 2.0% one or more silicate clays. In still other aspects of this embodiment, a grip enhancing composition disclosed herein comprises about 22% to about 24% of one or more chalks and about 1.7% to about 1.9% one or more silicate clays. In other aspects of this embodiment, a grip enhancing composition disclosed herein comprises about 23% of one or more chalks and about 1.8% one or more silicate clays. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, hypochlorous acid or free available chlorine, or any combination thereof.

In aspects of this embodiment, a grip enhancing composition disclosed herein comprises about 18% to about 28% magnesium carbonate and about 1.0% to about 2.5% sodium magnesium fluorosilicate or lithium magnesium sodium silicate. In other aspects of this embodiment, a grip enhancing composition disclosed herein comprises about 20% to about 26% magnesium carbonate and about 1.5% to about 2.1% sodium magnesium fluorosilicate or lithium magnesium sodium silicate. In yet other aspects of this embodiment, a grip enhancing composition disclosed herein comprises about 21% to about 25% magnesium carbonate and about 1.6% to about 2.0% sodium magnesium fluorosilicate or lithium magnesium sodium silicate. In still other aspects of this embodiment, a grip enhancing composition disclosed herein comprises about 22% to about 24% magnesium carbonate and about 1.7% to about 1.9% sodium magnesium fluorosilicate or lithium magnesium sodium silicate. In other aspects of this embodiment, a grip enhancing composition disclosed herein comprises about 23% magnesium carbonate and about 1.8% sodium magnesium fluorosilicate or lithium magnesium sodium silicate. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, hypochlorous acid or free available chlorine, or any combination thereof.

In one embodiment, a grip enhancing composition disclosed herein comprises one or more grip enhancers, one or more hydrophilic clays, and hypochlorous acid or free available chlorine. In aspects of this embodiment, a grip enhancing composition disclosed herein comprises about 18% to about 28% of one or more grip enhancers, about 1.0% to about 2.5% one or more hydrophilic clays and about 20 ppm to about 40 ppm hypochlorous acid or free available chlorine. In other aspects of this embodiment, a grip enhancing composition disclosed herein comprises about 20% to about 26% of one or more grip enhancers, about 1.5% to about 2.1% one or more hydrophilic clays and about 24 ppm to about 36 ppm hypochlorous acid or free available chlorine. In yet other aspects of this embodiment, a grip enhancing composition disclosed herein comprises about 21% to about 25% of one or more grip enhancers, about 1.6% to about 2.0% one or more hydrophilic clays and about 26 ppm to about 34 ppm hypochlorous acid or free available chlorine. In still other aspects of this embodiment, a grip enhancing composition disclosed herein comprises about 22% to about 24% of one or more grip enhancers, about 1.7% to about 1.9% one or more hydrophilic clays and about 28 ppm to about 32 ppm hypochlorous acid or free available chlorine. In other aspects of this embodiment, a grip enhancing composition disclosed herein comprises about 23% of one or more grip enhancers, about 1.8% one or more hydrophilic clays and about 30 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% of one or more chalks, about 1.0% to about 2.5% one or more silicate clays and about 20 ppm to about 40 ppm hypochlorous acid or free available chlorine. In other aspects of this embodiment, a grip enhancing composition disclosed herein comprises about 20% to about 26% of one or more chalks, about 1.5% to about 2.1% one or more silicate clays and about 24 ppm to about 36 ppm hypochlorous acid or free available chlorine. In yet other aspects of this embodiment, a grip enhancing composition disclosed herein comprises about 21% to about 25% of one or more chalks, about 1.6% to about 2.0% one or more silicate clays and about 26 ppm to about 34 ppm hypochlorous acid or free available chlorine. In still other aspects of this embodiment, a grip enhancing composition disclosed herein comprises about 22% to about 24% of one or more chalks, about 1.7% to about 1.9% one or more silicate clays and about 28 ppm to about 32 ppm hypochlorous acid or free available chlorine. In other aspects of this embodiment, a grip enhancing composition disclosed herein comprises about 23% of one or more chalks, about 1.8% one or more silicate clays and about 30 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% magnesium carbonate, about 1.0% to about 2.5% sodium magnesium fluorosilicate or lithium magnesium sodium silicate and about 20 ppm to about 40 ppm hypochlorous acid or free available chlorine. In other aspects of this embodiment, a grip enhancing composition disclosed herein comprises about 20% to about 26% magnesium carbonate, about 1.5% to about 2.1% sodium magnesium fluorosilicate or lithium magnesium sodium silicate and about 24 ppm to about 36 ppm hypochlorous acid or free available chlorine. In yet other aspects of this embodiment, a grip enhancing composition disclosed herein comprises about 21% to about 25% magnesium carbonate, about 1.6% to about 2.0% sodium magnesium fluorosilicate or lithium magnesium sodium silicate and about 26 ppm to about 34 ppm hypochlorous acid or free available chlorine. In still other aspects of this embodiment, a grip enhancing composition disclosed herein comprises about 22% to about 24% magnesium carbonate, about 1.7% to about 1.9% sodium magnesium fluorosilicate or lithium magnesium sodium silicate and about 28 ppm to about 32 ppm hypochlorous acid or free available chlorine. In other aspects of this embodiment, a grip enhancing composition disclosed herein comprises about 23% magnesium carbonate, about 1.8% sodium magnesium fluorosilicate or lithium magnesium sodium silicate and about 30 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% of one or more grip enhancers, about 1.0% to about 2.5% one or more hydrophilic clays and about 750 ppm to about 1,250 ppm hypochlorous acid or free available chlorine. In other aspects of this embodiment, a grip enhancing composition disclosed herein comprises about 20% to about 26% of one or more grip enhancers, about 1.5% to about 2.1% one or more hydrophilic clays and about 850 ppm to about 1,150 ppm hypochlorous acid or free available chlorine. In yet other aspects of this embodiment, a grip enhancing composition disclosed herein comprises about 21% to about 25% of one or more grip enhancers, about 1.6% to about 2.0% one or more hydrophilic clays and about 900 ppm to about 1,100 ppm hypochlorous acid or free available chlorine. In still other aspects of this embodiment, a grip enhancing composition disclosed herein comprises about 22% to about 24% of one or more grip enhancers, about 1.7% to about 1.9% one or more hydrophilic clays and about 950 ppm to about 1,050 ppm hypochlorous acid or free available chlorine. In other aspects of this embodiment, a grip enhancing composition disclosed herein comprises about 23% of one or more grip enhancers, about 1.8% one or more hydrophilic clays and about 1,000 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% of one or more chalks, about 1.0% to about 2.5% one or more silicate clays and about 750 ppm to about 1,250 ppm hypochlorous acid or free available chlorine. In other aspects of this embodiment, a grip enhancing composition disclosed herein comprises about 20% to about 26% of one or more chalks, about 1.5% to about 2.1% one or more silicate clays and about 850 ppm to about 1,150 ppm hypochlorous acid or free available chlorine. In yet other aspects of this embodiment, a grip enhancing composition disclosed herein comprises about 21% to about 25% of one or more chalks, about 1.6% to about 2.0% one or more silicate clays and about 900 ppm to about 1,100 ppm hypochlorous acid or free available chlorine. In still other aspects of this embodiment, a grip enhancing composition disclosed herein comprises about 22% to about 24% of one or more chalks, about 1.7% to about 1.9% one or more silicate clays and about 950 ppm to about 1,050 ppm hypochlorous acid or free available chlorine. In other aspects of this embodiment, a grip enhancing composition disclosed herein comprises about 23% of one or more chalks, about 1.8% one or more silicate clays and about 1,000 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% magnesium carbonate, about 1.0% to about 2.5% sodium magnesium fluorosilicate or lithium magnesium sodium silicate and about 750 ppm to about 1,250 ppm hypochlorous acid or free available chlorine. In other aspects of this embodiment, a grip enhancing composition disclosed herein comprises about 20% to about 26% magnesium carbonate, about 1.5% to about 2.1% sodium magnesium fluorosilicate or lithium magnesium sodium silicate and about 850 ppm to about 1,150 ppm hypochlorous acid or free available chlorine. In yet other aspects of this embodiment, a grip enhancing composition disclosed herein comprises about 21% to about 25% magnesium carbonate, about 1.6% to about 2.0% sodium magnesium fluorosilicate or lithium magnesium sodium silicate and about 900 ppm to about 1,100 ppm hypochlorous acid or free available chlorine. In still other aspects of this embodiment, a grip enhancing composition disclosed herein comprises about 22% to about 24% magnesium carbonate, about 1.7% to about 1.9% sodium magnesium fluorosilicate or lithium magnesium sodium silicate and about 950 ppm to about 1,050 ppm hypochlorous acid or free available chlorine. In other aspects of this embodiment, a grip enhancing composition disclosed herein comprises about 23% magnesium carbonate, about 1.8% sodium magnesium fluorosilicate or lithium magnesium sodium silicate and about 1,000 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% of one or more grip enhancers, about 1.0% to about 2.5% one or more hydrophilic clays and about 1,250 ppm to about 1,750 ppm hypochlorous acid or free available chlorine. In other aspects of this embodiment, a grip enhancing composition disclosed herein comprises about 20% to about 26% of one or more grip enhancers, about 1.5% to about 2.1% one or more hydrophilic clays and about 1,350 ppm to about 1,650 ppm hypochlorous acid or free available chlorine. In yet other aspects of this embodiment, a grip enhancing composition disclosed herein comprises about 21% to about 25% of one or more grip enhancers, about 1.6% to about 2.0% one or more hydrophilic clays and about 1,400 ppm to about 1,600 ppm hypochlorous acid or free available chlorine. In still other aspects of this embodiment, a grip enhancing composition disclosed herein comprises about 22% to about 24% of one or more grip enhancers, about 1.7% to about 1.9% one or more hydrophilic clays and about 1,450 ppm to about 1,550 ppm hypochlorous acid or free available chlorine. In other aspects of this embodiment, a grip enhancing composition disclosed herein comprises about 23% of one or more grip enhancers, about 1.8% one or more hydrophilic clays and about 1,500 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% of one or more chalks, about 1.0% to about 2.5% one or more silicate clays and about 1,250 ppm to about 1,750 ppm hypochlorous acid or free available chlorine. In other aspects of this embodiment, a grip enhancing composition disclosed herein comprises about 20% to about 26% of one or more chalks, about 1.5% to about 2.1% one or more silicate clays and about 1,350 ppm to about 1,650 ppm hypochlorous acid or free available chlorine. In yet other aspects of this embodiment, a grip enhancing composition disclosed herein comprises about 21% to about 25% of one or more chalks, about 1.6% to about 2.0% one or more silicate clays and about 1,400 ppm to about 1,600 ppm hypochlorous acid or free available chlorine. In still other aspects of this embodiment, a grip enhancing composition disclosed herein comprises about 22% to about 24% of one or more chalks, about 1.7% to about 1.9% one or more silicate clays and about 1,450 ppm to about 1,550 ppm hypochlorous acid or free available chlorine. In other aspects of this embodiment, a grip enhancing composition disclosed herein comprises about 23% of one or more chalks, about 1.8% one or more silicate clays and about 1,500 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% magnesium carbonate, about 1.0% to about 2.5% sodium magnesium fluorosilicate or lithium magnesium sodium silicate and about 1,250 ppm to about 1,750 ppm hypochlorous acid or free available chlorine. In other aspects of this embodiment, a grip enhancing composition disclosed herein comprises about 20% to about 26% magnesium carbonate, about 1.5% to about 2.1% sodium magnesium fluorosilicate or lithium magnesium sodium silicate and about 1,350 ppm to about 1,650 ppm hypochlorous acid or free available chlorine. In yet other aspects of this embodiment, a grip enhancing composition disclosed herein comprises about 21% to about 25% magnesium carbonate, about 1.6% to about 2.0% sodium magnesium fluorosilicate or lithium magnesium sodium silicate and about 1,400 ppm to about 1,600 ppm hypochlorous acid or free available chlorine. In still other aspects of this embodiment, a grip enhancing composition disclosed herein comprises about 22% to about 24% magnesium carbonate, about 1.7% to about 1.9% sodium magnesium fluorosilicate or lithium magnesium sodium silicate and about 1,450 ppm to about 1,550 ppm hypochlorous acid or free available chlorine. In other aspects of this embodiment, a grip enhancing composition disclosed herein comprises about 23% magnesium carbonate, about 1.8% sodium magnesium fluorosilicate or lithium magnesium sodium silicate and about 1,500 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% of one or more grip enhancers, about 1.0% to about 2.5% one or more hydrophilic clays and about 1,750 ppm to about 2,250 ppm hypochlorous acid or free available chlorine. In other aspects of this embodiment, a grip enhancing composition disclosed herein comprises about 20% to about 26% of one or more grip enhancers, about 1.5% to about 2.1% one or more hydrophilic clays and about 1,850 ppm to about 2,150 ppm hypochlorous acid or free available chlorine. In yet other aspects of this embodiment, a grip enhancing composition disclosed herein comprises about 21% to about 25% of one or more grip enhancers, about 1.6% to about 2.0% one or more hydrophilic clays and about 1,900 ppm to about 2,100 ppm hypochlorous acid or free available chlorine. In still other aspects of this embodiment, a grip enhancing composition disclosed herein comprises about 22% to about 24% of one or more grip enhancers, about 1.7% to about 1.9% one or more hydrophilic clays and about 1,950 ppm to about 2,050 ppm hypochlorous acid or free available chlorine. In other aspects of this embodiment, a grip enhancing composition disclosed herein comprises about 23% of one or more grip enhancers, about 1.8% one or more hydrophilic clays and about 2,000 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% of one or more chalks, about 1.0% to about 2.5% one or more silicate clays and about 1,750 ppm to about 2,250 ppm hypochlorous acid or free available chlorine. In other aspects of this embodiment, a grip enhancing composition disclosed herein comprises about 20% to about 26% of one or more chalks, about 1.5% to about 2.1% one or more silicate clays and about 1,850 ppm to about 2,150 ppm hypochlorous acid or free available chlorine. In yet other aspects of this embodiment, a grip enhancing composition disclosed herein comprises about 21% to about 25% of one or more chalks, about 1.6% to about 2.0% one or more silicate clays and about 1,900 ppm to about 2,100 ppm hypochlorous acid or free available chlorine. In still other aspects of this embodiment, a grip enhancing composition disclosed herein comprises about 22% to about 24% of one or more chalks, about 1.7% to about 1.9% one or more silicate clays and about 1,950 ppm to about 2,050 ppm hypochlorous acid or free available chlorine. In other aspects of this embodiment, a grip enhancing composition disclosed herein comprises about 23% of one or more chalks, about 1.8% one or more silicate clays and about 2,000 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% magnesium carbonate, about 1.0% to about 2.5% sodium magnesium fluorosilicate or lithium magnesium sodium silicate and about 1,750 ppm to about 2,250 ppm hypochlorous acid or free available chlorine. In other aspects of this embodiment, a grip enhancing composition disclosed herein comprises about 20% to about 26% magnesium carbonate, about 1.5% to about 2.1% sodium magnesium fluorosilicate or lithium magnesium sodium silicate and about 1,850 ppm to about 2,150 ppm hypochlorous acid or free available chlorine. In yet other aspects of this embodiment, a grip enhancing composition disclosed herein comprises about 21% to about 25% magnesium carbonate, about 1.6% to about 2.0% sodium magnesium fluorosilicate or lithium magnesium sodium silicate and about 1,900 ppm to about 2,100 ppm hypochlorous acid or free available chlorine. In still other aspects of this embodiment, a grip enhancing composition disclosed herein comprises about 22% to about 24% magnesium carbonate, about 1.7% to about 1.9% sodium magnesium fluorosilicate or lithium magnesium sodium silicate and about 1,950 ppm to about 2,050 ppm hypochlorous acid or free available chlorine. In other aspects of this embodiment, a grip enhancing composition disclosed herein comprises about 23% magnesium carbonate, about 1.8% sodium magnesium fluorosilicate or lithium magnesium sodium silicate and about 2,000 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In one embodiment, a grip enhancing composition disclosed herein comprises one or more grip enhancers, one or more hydrophilic clays, one or more polysaccharides, and hypochlorous acid or free available chlorine. In aspects of this embodiment, a grip enhancing composition disclosed herein comprises about 18% to about 28% of one or more grip enhancers, about 0.5% to about 1.5% of one or more hydrophilic clays, about 1.0% to about 2.5% of one or more polysaccharides, and about 750 ppm to about 1,250 ppm hypochlorous acid or free available chlorine. In other aspects of this embodiment, a grip enhancing composition disclosed herein comprises about 20% to about 26% of one or more grip enhancers, about 0.7% to about 1.3% of one or more hydrophilic clays, about 1.7% to about 2.3% of one or more polysaccharides, and about 850 ppm to about 1,150 ppm hypochlorous acid or free available chlorine. In yet other aspects of this embodiment, a grip enhancing composition disclosed herein comprises about 21% to about 25% of one or more grip enhancers, about 0.8% to about 1.2% of one or more hydrophilic clays, about 1.8% to about 2.2% of one or more polysaccharides, and about 900 ppm to about 1,100 ppm hypochlorous acid or free available chlorine. In still other aspects of this embodiment, a grip enhancing composition disclosed herein comprises about 22% to about 24% of one or more grip enhancers, about 0.9% to about 1.1% of one or more hydrophilic clays, about 1.9% to about 2.1% of one or more polysaccharides, and about 950 ppm to about 1,050 ppm hypochlorous acid or free available chlorine. In other aspects of this embodiment, a grip enhancing composition disclosed herein comprises about 23% of one or more grip enhancers, about 1.0% of one or more hydrophilic clays, about 2.0% of one or more polysaccharides, and about 1,000 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% of one or more chalks, about 0.5% to about 1.5% of one or more silicate clays, about 1.0% to about 2.5% of one or more cellulose ether derivatives, and about 750 ppm to about 1,250 ppm hypochlorous acid or free available chlorine. In other aspects of this embodiment, a grip enhancing composition disclosed herein comprises about 20% to about 26% of one or more chalks, about 0.7% to about 1.3% of one or more silicate clays, about 1.7% to about 2.3% of one or more cellulose ether derivatives, and about 850 ppm to about 1,150 ppm hypochlorous acid or free available chlorine. In yet other aspects of this embodiment, a grip enhancing composition disclosed herein comprises about 21% to about 25% of one or more chalks, about 0.8% to about 1.2% of one or more silicate clays, about 1.8% to about 2.2% of one or more cellulose ether derivatives, and about 900 ppm to about 1,100 ppm hypochlorous acid or free available chlorine. In still other aspects of this embodiment, a grip enhancing composition disclosed herein comprises about 22% to about 24% of one or more chalks, about 0.9% to about 1.1% of one or more silicate clays, about 1.9% to about 2.1% of one or more cellulose ether derivatives, and about 950 ppm to about 1,050 ppm hypochlorous acid or free available chlorine. In other aspects of this embodiment, a grip enhancing composition disclosed herein comprises about 23% of one or more chalks, about 1.0% of one or more silicate clays, about 2.0% of one or more cellulose ether derivatives, and about 1,000 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% magnesium carbonate, about 0.5% to about 1.5% sodium magnesium fluorosilicate or lithium magnesium sodium silicate, about 1.0% to about 2.5% hydroxyethyl cellulose, and about 750 ppm to about 1,250 ppm hypochlorous acid or free available chlorine. In other aspects of this embodiment, a grip enhancing composition disclosed herein comprises about 20% to about 26% magnesium carbonate, about 0.7% to about 1.3% sodium magnesium fluorosilicate or lithium magnesium sodium silicate, about 1.7% to about 2.3% hydroxyethyl cellulose, and about 850 ppm to about 1,150 ppm hypochlorous acid or free available chlorine. In yet other aspects of this embodiment, a grip enhancing composition disclosed herein comprises about 21% to about 25% magnesium carbonate, about 0.8% to about 1.2% sodium magnesium fluorosilicate or lithium magnesium sodium silicate, about 1.8% to about 2.2% hydroxyethyl cellulose, and about 900 ppm to about 1,100 ppm hypochlorous acid or free available chlorine. In still other aspects of this embodiment, a grip enhancing composition disclosed herein comprises about 22% to about 24% magnesium carbonate, about 0.9% to about 1.1% sodium magnesium fluorosilicate or lithium magnesium sodium silicate, about 1.9% to about 2.1% hydroxyethyl cellulose, and about 950 ppm to about 1,050 ppm hypochlorous acid or free available chlorine. In other aspects of this embodiment, a grip enhancing composition disclosed herein comprises about 23% magnesium carbonate, about 1.0% sodium magnesium fluorosilicate or lithium magnesium sodium silicate, about 2.0% hydroxyethyl cellulose, and about 1,000 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% of one or more grip enhancers, about 0.5% to about 1.5% of one or more hydrophilic clays, about 1.0% to about 2.5% of one or more polysaccharides, and about 1,250 ppm to about 1,750 ppm hypochlorous acid or free available chlorine. In other aspects of this embodiment, a grip enhancing composition disclosed herein comprises about 20% to about 26% of one or more grip enhancers, about 0.7% to about 1.3% of one or more hydrophilic clays, about 1.7% to about 2.3% of one or more polysaccharides, and about 1,350 ppm to about 1,650 ppm hypochlorous acid or free available chlorine. In yet other aspects of this embodiment, a grip enhancing composition disclosed herein comprises about 21% to about 25% of one or more grip enhancers, about 0.8% to about 1.2% of one or more hydrophilic clays, about 1.8% to about 2.2% of one or more polysaccharides, and about 1,400 ppm to about 1,600 ppm hypochlorous acid or free available chlorine. In still other aspects of this embodiment, a grip enhancing composition disclosed herein comprises about 22% to about 24% of one or more grip enhancers, about 0.9% to about 1.1% of one or more hydrophilic clays, about 1.9% to about 2.1% of one or more polysaccharides, and about 1,450 ppm to about 1,550 ppm hypochlorous acid or free available chlorine. In other aspects of this embodiment, a grip enhancing composition disclosed herein comprises about 23% of one or more grip enhancers, about 1.0% of one or more hydrophilic clays, about 2.0% of one or more polysaccharides, and about 1,500 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% of one or more chalks, about 0.5% to about 1.5% of one or more silicate clays, about 1.0% to about 2.5% of one or more cellulose ether derivatives, and about 1,250 ppm to about 1,750 ppm hypochlorous acid or free available chlorine. In other aspects of this embodiment, a grip enhancing composition disclosed herein comprises about 20% to about 26% of one or more chalks, about 0.7% to about 1.3% of one or more silicate clays, about 1.7% to about 2.3% of one or more cellulose ether derivatives, and about 1,350 ppm to about 1,650 ppm hypochlorous acid or free available chlorine. In yet other aspects of this embodiment, a grip enhancing composition disclosed herein comprises about 21% to about 25% of one or more chalks, about 0.8% to about 1.2% of one or more silicate clays, about 1.8% to about 2.2% of one or more cellulose ether derivatives, and about 1,400 ppm to about 1,600 ppm hypochlorous acid or free available chlorine. In still other aspects of this embodiment, a grip enhancing composition disclosed herein comprises about 22% to about 24% of one or more chalks, about 0.9% to about 1.1% of one or more silicate clays, about 1.9% to about 2.1% of one or more cellulose ether derivatives, and about 1,450 ppm to about 1,550 ppm hypochlorous acid or free available chlorine. In other aspects of this embodiment, a grip enhancing composition disclosed herein comprises about 23% of one or more chalks, about 1.0% of one or more silicate clays, about 2.0% of one or more cellulose ether derivatives, and about 1,500 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% magnesium carbonate, about 0.5% to about 1.5% sodium magnesium fluorosilicate or lithium magnesium sodium silicate, about 1.0% to about 2.5% hydroxyethyl cellulose, and about 1,250 ppm to about 1,750 ppm hypochlorous acid or free available chlorine. In other aspects of this embodiment, a grip enhancing composition disclosed herein comprises about 20% to about 26% magnesium carbonate, about 0.7% to about 1.3% sodium magnesium fluorosilicate or lithium magnesium sodium silicate, about 1.7% to about 2.3% hydroxyethyl cellulose, and about 1,350 ppm to about 1,650 ppm hypochlorous acid or free available chlorine. In yet other aspects of this embodiment, a grip enhancing composition disclosed herein comprises about 21% to about 25% magnesium carbonate, about 0.8% to about 1.2% sodium magnesium fluorosilicate or lithium magnesium sodium silicate, about 1.8% to about 2.2% hydroxyethyl cellulose, and about 1,400 ppm to about 1,600 ppm hypochlorous acid or free available chlorine. In still other aspects of this embodiment, a grip enhancing composition disclosed herein comprises about 22% to about 24% magnesium carbonate, about 0.9% to about 1.1% sodium magnesium fluorosilicate or lithium magnesium sodium silicate, about 1.9% to about 2.1% hydroxyethyl cellulose, and about 1,450 ppm to about 1,550 ppm hypochlorous acid or free available chlorine. In other aspects of this embodiment, a grip enhancing composition disclosed herein comprises about 23% magnesium carbonate, about 1.0% sodium magnesium fluorosilicate or lithium magnesium sodium silicate, about 2.0% hydroxyethyl cellulose, and about 1,500 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% of one or more grip enhancers, about 0.5% to about 1.5% of one or more hydrophilic clays, about 1.0% to about 2.5% of one or more polysaccharides, and about 1,750 ppm to about 2,250 ppm hypochlorous acid or free available chlorine. In other aspects of this embodiment, a grip enhancing composition disclosed herein comprises about 20% to about 26% of one or more grip enhancers, about 0.7% to about 1.3% of one or more hydrophilic clays, about 1.7% to about 2.3% of one or more polysaccharides, and about 1,850 ppm to about 2,150 ppm hypochlorous acid or free available chlorine. In yet other aspects of this embodiment, a grip enhancing composition disclosed herein comprises about 21% to about 25% of one or more grip enhancers, about 0.8% to about 1.2% of one or more hydrophilic clays, about 1.8% to about 2.2% of one or more polysaccharides, and about 1,900 ppm to about 2,100 ppm hypochlorous acid or free available chlorine. In still other aspects of this embodiment, a grip enhancing composition disclosed herein comprises about 22% to about 24% of one or more grip enhancers, about 0.9% to about 1.1% of one or more hydrophilic clays, about 1.9% to about 2.1% of one or more polysaccharides, and about 1,950 ppm to about 2,050 ppm hypochlorous acid or free available chlorine. In other aspects of this embodiment, a grip enhancing composition disclosed herein comprises about 23% of one or more grip enhancers, about 1.0% of one or more hydrophilic clays, about 2.0% of one or more polysaccharides, and about 2,000 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% of one or more chalks, about 0.5% to about 1.5% of one or more silicate clays, about 1.0% to about 2.5% of one or more cellulose ether derivatives, and about 1,750 ppm to about 2,250 ppm hypochlorous acid or free available chlorine. In other aspects of this embodiment, a grip enhancing composition disclosed herein comprises about 20% to about 26% of one or more chalks, about 0.7% to about 1.3% of one or more silicate clays, about 1.7% to about 2.3% of one or more cellulose ether derivatives, and about 1,850 ppm to about 2,150 ppm hypochlorous acid or free available chlorine. In yet other aspects of this embodiment, a grip enhancing composition disclosed herein comprises about 21% to about 25% of one or more chalks, about 0.8% to about 1.2% of one or more silicate clays, about 1.8% to about 2.2% of one or more cellulose ether derivatives, and about 1,900 ppm to about 2,100 ppm hypochlorous acid or free available chlorine. In still other aspects of this embodiment, a grip enhancing composition disclosed herein comprises about 22% to about 24% of one or more chalks, about 0.9% to about 1.1% of one or more silicate clays, about 1.9% to about 2.1% of one or more cellulose ether derivatives, and about 1,950 ppm to about 2,050 ppm hypochlorous acid or free available chlorine. In other aspects of this embodiment, a grip enhancing composition disclosed herein comprises about 23% of one or more chalks, about 1.0% of one or more silicate clays, about 2.0% of one or more cellulose ether derivatives, and about 2,000 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% magnesium carbonate, about 0.5% to about 1.5% sodium magnesium fluorosilicate or lithium magnesium sodium silicate, about 1.0% to about 2.5% hydroxyethyl cellulose, and about 1,750 ppm to about 2,250 ppm hypochlorous acid or free available chlorine. In other aspects of this embodiment, a grip enhancing composition disclosed herein comprises about 20% to about 26% magnesium carbonate, about 0.7% to about 1.3% sodium magnesium fluorosilicate or lithium magnesium sodium silicate, about 1.7% to about 2.3% hydroxyethyl cellulose, and about 1,850 ppm to about 2,150 ppm hypochlorous acid or free available chlorine. In yet other aspects of this embodiment, a grip enhancing composition disclosed herein comprises about 21% to about 25% magnesium carbonate, about 0.8% to about 1.2% sodium magnesium fluorosilicate or lithium magnesium sodium silicate, about 1.8% to about 2.2% hydroxyethyl cellulose, and about 1,900 ppm to about 2,100 ppm hypochlorous acid or free available chlorine. In still other aspects of this embodiment, a grip enhancing composition disclosed herein comprises about 22% to about 24% magnesium carbonate, about 0.9% to about 1.1% sodium magnesium fluorosilicate or lithium magnesium sodium silicate, about 1.9% to about 2.1% hydroxyethyl cellulose, and about 1,950 ppm to about 2,050 ppm hypochlorous acid or free available chlorine. In other aspects of this embodiment, a grip enhancing composition disclosed herein comprises about 23% magnesium carbonate, about 1.0% sodium magnesium fluorosilicate or lithium magnesium sodium silicate, about 2.0% hydroxyethyl cellulose, and about 2.000 ppm hypochlorous acid or free available chlorine. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In one embodiment, a grip enhancing composition disclosed herein comprises one or more grip enhancers, one or more hydrophilic clays, one or more polysaccharides, and electrolytic alkaline water. In aspects of this embodiment, a grip enhancing composition disclosed herein comprises about 18% to about 28% of one or more grip enhancers, about 0.5% to about 1.5% of one or more hydrophilic clays, about 1.0% to about 2.5% of one or more polysaccharides, and electrolytic alkaline water. In other aspects of this embodiment, a grip enhancing composition disclosed herein comprises about 20% to about 26% of one or more grip enhancers, about 0.7% to about 1.3% of one or more hydrophilic clays, about 1.7% to about 2.3% of one or more polysaccharides, and electrolytic alkaline water. In yet other aspects of this embodiment, a grip enhancing composition disclosed herein comprises about 21% to about 25% of one or more grip enhancers, about 0.8% to about 1.2% of one or more hydrophilic clays, about 1.8% to about 2.2% of one or more polysaccharides, and electrolytic alkaline water. In still other aspects of this embodiment, a grip enhancing composition disclosed herein comprises about 22% to about 24% of one or more grip enhancers, about 0.9% to about 1.1% of one or more hydrophilic clays, about 1.9% to about 2.1% of one or more polysaccharides, and electrolytic alkaline water. In other aspects of this embodiment, a grip enhancing composition disclosed herein comprises about 23% of one or more grip enhancers, about 1.0% of one or more hydrophilic clays, about 2.0% of one or more polysaccharides, and electrolytic alkaline water. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% of one or more chalks, about 0.5% to about 1.5% of one or more silicate clays, about 1.0% to about 2.5% of one or more cellulose ether derivatives, and electrolytic alkaline water. In other aspects of this embodiment, a grip enhancing composition disclosed herein comprises about 20% to about 26% of one or more chalks, about 0.7% to about 1.3% of one or more silicate clays, about 1.7% to about 2.3% of one or more cellulose ether derivatives, and electrolytic alkaline water. In yet other aspects of this embodiment, a grip enhancing composition disclosed herein comprises about 21% to about 25% of one or more chalks, about 0.8% to about 1.2% of one or more silicate clays, about 1.8% to about 2.2% of one or more cellulose ether derivatives, and electrolytic alkaline water. In still other aspects of this embodiment, a grip enhancing composition disclosed herein comprises about 22% to about 24% of one or more chalks, about 0.9% to about 1.1% of one or more silicate clays, about 1.9% to about 2.1% of one or more cellulose ether derivatives, and electrolytic alkaline water. In other aspects of this embodiment, a grip enhancing composition disclosed herein comprises about 23% of one or more chalks, about 1.0% of one or more silicate clays, about 2.0% of one or more cellulose ether derivatives, and electrolytic alkaline water. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

In aspects of this embodiment, a grip enhancing composition disclosed herein comprises about 18% to about 28% magnesium carbonate, about 0.5% to about 1.5% sodium magnesium fluorosilicate or lithium magnesium sodium silicate, about 1.0% to about 2.5% hydroxyethyl cellulose, and electrolytic alkaline water. In other aspects of this embodiment, a grip enhancing composition disclosed herein comprises about 20% to about 26% magnesium carbonate, about 0.7% to about 1.3% sodium magnesium fluorosilicate or lithium magnesium sodium silicate, about 1.7% to about 2.3% hydroxyethyl cellulose, and electrolytic alkaline water. In yet other aspects of this embodiment, a grip enhancing composition disclosed herein comprises about 21% to about 25% magnesium carbonate, about 0.8% to about 1.2% sodium magnesium fluorosilicate or lithium magnesium sodium silicate, about 1.8% to about 2.2% hydroxyethyl cellulose, and electrolytic alkaline water. In still other aspects of this embodiment, a grip enhancing composition disclosed herein comprises about 22% to about 24% magnesium carbonate, about 0.9% to about 1.1% sodium magnesium fluorosilicate or lithium magnesium sodium silicate, about 1.9% to about 2.1% hydroxyethyl cellulose, and electrolytic alkaline water. In other aspects of this embodiment, a grip enhancing composition disclosed herein comprises about 23% magnesium carbonate, about 1.0% sodium magnesium fluorosilicate or lithium magnesium sodium silicate, about 2.0% hydroxyethyl cellulose, and electrolytic alkaline water. In yet other aspects of this embodiment, a grip enhancing composition disclosed herein does not include an alcohol. In still other aspects of this embodiment, a grip enhancing composition disclosed herein can further include one or more carriers, one or more fragrances, one or more antimicrobials, one or more silicones, or any combination thereof.

Aspects of the present specification disclose a kit. In one embodiment, the kit can comprise a container including a grip enhancing composition disclosed herein. In another embodiment, a kit can comprise one or more containers, each container including an individual component or more than one individual component disclosed herein in combination. For example, a kit can comprise one container including a composition comprising one or more grip enhancers and one or more hydrophilic clays and a second container including ascorbic acid composition. As another example, a kit can comprise one container including a composition comprising one or more grip enhancers and one or more hydrophilic clays and a second container including a composition comprising hypochlorous acid or free available chlorine. As yet another example, a kit can comprise one container including a composition comprising one or more grip enhancers and one or more hydrophilic clays, a second container including ascorbic acid composition, and a third container including a composition comprising hypochlorous acid or free available chlorine. The remainder of the components of a grip enhancing composition disclosed herein may be included in either the first or second container, or may be separately including in at least a third container. Packaging of individual components into separate container can assist in prolonging the stability of the individual components, and thus shelf life of the product.

A kit disclosed herein can comprise a delivery or application system. The delivery or application system of the kit are useful for applying a grip enhancing composition disclosed herein, and/or individual components disclosed herein to a site of interest, such as, e.g., a surface of a device disclosed herein. A delivery or application system disclosed herein, includes, without limitation, one or more of an applicator brush, porous foam swab or pad, hollow tube, dipstick, or a combination thereof. In an embodiment, a kit comprises a single delivery or application system. In another embodiment, a kit comprises a plurality delivery or application systems. For example, in a 30-day supply kit, there can be 30 delivery or application systems, such that there is one delivery or application system per day for 30 days. Alternately, there can be 2, 10, 20, 30, 40, 50, 60, 90, 120, etc. delivery or application systems per kit. Within the kit, the delivery or application system may be packaged individually, or in sets of 2 or more. The delivery or application system can be packaged such that it remains sterile until use. In certain embodiments, a delivery or application system disclosed herein can be packaged in plastic sheaths. Further, to prevent contamination, delivery or application system disclosed herein is preferably single-use, disposable delivery or application system.

The kit can also comprise a set of instructions. The instructions may include information useful to the end user such as how to use a delivery or application system to apply a grip enhancing composition and/or individual components disclosed herein, and/or how often to apply a grip enhancing composition and/or individual components disclosed herein. In addition, such instructions may include information regarding how to mix the individual components disclosed herein to form a grip enhancing composition disclosed herein. Such instructions can indicate that mixing should be done at a certain time before application, such as, e.g., just prior to use. Instructions disclosed herein may also include information regarding how to apply the individual components disclosed herein directly to a site of interest, such as, e.g., a surface of a device disclosed herein, and in what order the individual components should be applied to such sites of interest.

The contents of the kit, including the container including a grip enhancing composition or component disclosed herein, the delivery or application system, and instructions, are enclosed in an outer casing. The outer casing can be a box, a sealed bag, a foil pouch, etc. In certain embodiments, the delivery system, container and instructions are enclosed in a box. In other embodiments of the kit, the container and instructions are contained in a first box, the delivery system is contained in a second box, and the first and second box are contained together in a third box.

Aspects of the present specification disclose a method to improve the grip capacity of an individual. Other aspects of the present specification disclose a grip enhancing composition disclosed herein for use to improve the grip capacity of an individual. Yet other aspects of the present specification disclose a use of a grip enhancing composition disclosed herein to improve the grip capacity of an individual. Still other aspects of the present specification disclose a use of a grip enhancing composition disclosed herein in the manufacture of a medicament to improve the grip capacity of an individual.

In one embodiment, the disclosed method or use comprises applying a grip enhancing composition disclosed herein to a skin region of an individual. A skin region includes, without limitation, the face, forehead, lips, scalp, neck, shoulder, arms, elbows, wrists, hands, palms of hands, fingers, thighs, legs, knees, feet, soles of feet, chest, breast, back, groin, buttocks, and the like. In an aspect of this embodiment, the disclosed method or use comprises applying a grip enhancing composition disclosed herein to a first palm surface of a first hand of an individual, a second palm surface of a second hand of an individual, or a combination thereof. Such application to the skin region or palm surface of the individual restores, maintains and/or improves the grip capacity of the individual, allowing the individual to more securely grip an object.

Aspects of the present specification disclose a method of providing protection to a skin region of an individual. Other aspects of the present specification disclose a grip enhancing composition disclosed herein for use in providing protection to a skin region of an individual. Yet other aspects of the present specification disclose a use of a grip enhancing composition disclosed herein to provide protection to a skin region of an individual. Still other aspects of the present specification disclose a use of a grip enhancing composition disclosed herein in the manufacture of a medicament to provide protection to a skin region of an individual.

In another aspect of this embodiment, the disclosed method or use comprises applying a grip enhancing composition disclosed herein to a first foot surface of a first foot of an individual, a second foot surface of a second foot of an individual, or a combination thereof. Such application to the foot surface of the individual protects the foot surface of the individual against friction, allowing the individual to more comfortably wear shoes.

Aspects of the present specification disclose a method to improve the grip capacity of a surface of an object. Other aspects of the present specification disclose a grip enhancing composition disclosed herein for use to improve the grip capacity of a surface of an object. Yet other aspects of the present specification disclose a use of a grip enhancing composition disclosed herein to improve the grip capacity of a surface of an object. Still other aspects of the present specification disclose a use of a grip enhancing composition disclosed herein in the manufacture of a medicament to improve the grip capacity of a surface of an object.

In one embodiment, the disclosed method or use comprises applying a grip enhancing composition disclosed herein to a surface of an object. In an aspect of this embodiment, the disclosed method or use comprises applying a grip enhancing composition disclosed herein to a surface of a gripping portion of an object. Such application to the surface restores, maintains and/or improves the grip capacity of the individual. An object includes, without limitation, any man-made or natural object, such as, e.g., a piece of equipment, apparatus, implement, a rock, a boulder, a tree, a branch, or other natural formation, or other object comprising a gripping portion that an individual desire to grip or contact securely in order to competently, proficiently and/or safely execute an activity being performed using the object. A surface disclosed herein is typically a griping portion of an object disclosed herein. Non-limiting examples of an object include a bar, a barbell, a dumbbell, a kettlebell, an uneven bar, a ring, a balance beam, a pommel horse, a vault, a mat, a floor, a pole, a wrist strap, an inside glove surface, an outside glove surface, a baseball bat, a softball bat, a cricket bat, a golf club, an ice hockey stick, a field hockey stick, a tennis racket, a racquetball racket, a shoe, a javelin, a spear, a gun, a tool, a rope, a disc, a frisbee, a saddle, an oar, a piece of equipment, a cable, a rock and a tree branch.

A method or use disclosed herein applies a composition disclosed herein to an individual. An individual refers to any mammal including a human, a horse, a cow, a sheep, a dog and a cat. As such, a method or use disclosed herein is for human use as well as veterinarian use.

A grip enhancing composition disclosed herein can be applied according to a method or use disclosed herein to a skin region. Application of a grip enhancing compositions disclosed herein can be by rubbing, spreading, pouring, sprinkling, or spraying on, or otherwise applied to a skin region of an individual in accordance with the US FD&C Act, § 201(i). A grip enhancing composition disclosed herein can be applied by introducing the composition into or onto a solid support such as, e.g., a wipe, a patch, a strip, a towelette, a towel, a mitt, a glove, or a mask and then applying the composition to a skin or hair region. Application of a grip enhancing compositions disclosed herein can be by rubbing, spreading, pouring, sprinkling, or spraying on, or otherwise applied to a surface of an object. A grip enhancing composition disclosed herein can be applied by using a delivery device, such as, e.g., an aerosol dispenser, a pump spray, a trigger spray or a squeeze bottle, to apply the composition to a skin or hair region.

A grip enhancing composition disclosed herein can be applied according to a method or use disclosed herein as often as needed and/or desired. A grip enhancing composition disclosed herein can be applied daily, every other day, every third of day, once a week, multiple times per week, once a month, multiple times per month, once a year or multiple times per year, as desired. A grip enhancing composition disclosed herein can be applied multiple times per day, e.g., twice a day, three times a day, four time a day, five times a day, six times a day or as often as desired.

Aspects of the present specification disclose a method to restore, maintain, and/or improve a grip capacity. Grip capacity is the ability to grasp, grab, clutch, clasp or otherwise hold an object. In one embodiment, an improved grip capacity is assessed by the ability of an individual to competently, proficiently and/or safely execute an activity being performed using an object disclosed herein. Grip capacity can be assessed by measuring grip strength, grip force, grip pressure, grip friction, grip slippage, and/or slippage speed, using an appropriate instrument, such as, e.g., a sphygmomanometer, a Jamar dynamometer, a tribometer, a potentiometric force sensor, or a force sensing resistor (FSR) sensor, while utilizing standardized measurement procedures.

In aspects of this embodiment, a grip capacity of an individual as measured by grip pressure can be, e.g., about 100 mmHg, about 125 mmHg, about 150 mmHg, about 175 mmHg, about 200 mmHg, about 225 mmHg, about 250 mmHg, about 275 mmHg, or about 300 mmHg. In other aspects of this embodiment, a grip capacity of an individual as measured by grip pressure can be, e.g., at least 100 mmHg, at least 125 mmHg, at least 150 mmHg, at least 175 mmHg, at least 200 mmHg, at least 225 mmHg, at least 250 mmHg, at least 275 mmHg, or at least 300 mmHg. In yet other aspects of this embodiment, a grip capacity of an individual as measured by grip pressure can be, e.g., at most 100 mmHg, at most 125 mmHg, at most 150 mmHg, at most 175 mmHg, at most 200 mmHg, at most 225 mmHg, at most 250 mmHg, at most 275 mmHg, or at most 300 mmHg. In still other aspects of this embodiment, a grip capacity of an individual as measured by grip pressure can be from, e.g., about 100 mmHg to about 125 mmHg, about 100 mmHg to about 150 mmHg, about 100 mmHg to about 175 mmHg, about 100 mmHg to about 200 mmHg, about 100 mmHg to about 225 mmHg, about 100 mmHg to about 250 mmHg, about 100 mmHg to about 275 mmHg, about 100 mmHg to about 300 mmHg, about 125 mmHg to about 150 mmHg, about 125 mmHg to about 175 mmHg, about 125 mmHg to about 200 mmHg, about 125 mmHg to about 225 mmHg, about 125 mmHg to about 250 mmHg, about 125 mmHg to about 275 mmHg, about 125 mmHg to about 300 mmHg, about 150 mmHg to about 175 mmHg, about 150 mmHg to about 200 mmHg, about 150 mmHg to about 225 mmHg, about 150 mmHg to about 250 mmHg, about 150 mmHg to about 275 mmHg, about 150 mmHg to about 300 mmHg, about 175 mmHg to about 200 mmHg, about 175 mmHg to about 225 mmHg, about 175 mmHg to about 250 mmHg, about 175 mmHg to about 275 mmHg, about 175 mmHg to about 300 mmHg, about 200 mmHg to about 225 mmHg, about 200 mmHg to about 250 mmHg, about 200 mmHg to about 275 mmHg, about 200 mmHg to about 300 mmHg, about 225 mmHg to about 250 mmHg, about 225 mmHg to about 275 mmHg, about 225 mmHg to about 300 mmHg, about 250 mmHg to about 275 mmHg, about 250 mmHg to about 300 mmHg, or about 275 mmHg to about 300 mmHg.

In aspects of this embodiment, a grip capacity of an individual as measured by grip pressure can be improved by, e.g., about 10 mmHg, about 20 mmHg, about 30 mmHg, about 40 mmHg, about 50 mmHg, about 60 mmHg, about 70 mmHg, about 80 mmHg, about 90 mmHg, or about 100 mmHg. In other aspects of this embodiment, a grip capacity of an individual can be improved by, e.g., at least 10 mmHg, at least 20 mmHg, at least 30 mmHg, at least 40 mmHg, at least 50 mmHg, at least 60 mmHg, at least 70 mmHg, at least 80 mmHg, at least 90 mmHg, or at least 100 mmHg. In yet other aspects of this embodiment, a grip capacity of an individual as measured by grip pressure can be improved by, e.g., at most 10 mmHg, at most 20 mmHg, at most 30 mmHg, at most 40 mmHg, at most 50 mmHg, at most 60 mmHg, at most 70 mmHg, at most 80 mmHg, at most 90 mmHg, or at most 100 mmHg. In still other aspects of this embodiment, a grip capacity of an individual as measured by grip pressure can be improved by, e.g., about 10 mmHg to about 20 mmHg, about 10 mmHg to about 30 mmHg, about 10 mmHg to about 40 mmHg, about 10 mmHg to about 50 mmHg, about 10 mmHg to about 60 mmHg, about 10 mmHg to about 70 mmHg, about 10 mmHg to about 80 mmHg, about 10 mmHg to about 90 mmHg, about 10 mmHg to about 100 mmHg, about 20 mmHg to about 30 mmHg, about 20 mmHg to about 40 mmHg, about 20 mmHg to about 50 mmHg, about 20 mmHg to about 60 mmHg, about 20 mmHg to about 70 mmHg, about 20 mmHg to about 80 mmHg, about 20 mmHg to about 90 mmHg, about 20 mmHg to about 100 mmHg, about 30 mmHg to about 40 mmHg, about 30 mmHg to about 50 mmHg, about 30 mmHg to about 60 mmHg, about 30 mmHg to about 70 mmHg, about 30 mmHg to about 80 mmHg, about 30 mmHg to about 90 mmHg, about 30 mmHg to about 100 mmHg, about 40 mmHg to about 50 mmHg, about 40 mmHg to about 60 mmHg, about 40 mmHg to about 70 mmHg, about 40 mmHg to about 80 mmHg, about 40 mmHg to about 90 mmHg, about 40 mmHg to about 100 mmHg, about 50 mmHg to about 60 mmHg, about 50 mmHg to about 70 mmHg, about 50 mmHg to about 80 mmHg, about 50 mmHg to about 90 mmHg, about 50 mmHg to about 100 mmHg, about 60 mmHg to about 70 mmHg, about 60 mmHg to about 80 mmHg, about 60 mmHg to about 90 mmHg, about 60 mmHg to about 100 mmHg, about 70 mmHg to about 80 mmHg, about 70 mmHg to about 90 mmHg, about 70 mmHg to about 100 mmHg, about 80 mmHg to about 90 mmHg, about 80 mmHg to about 100 mmHg, or about 90 mmHg to about 100 mmHg.

In aspects of this embodiment, a grip capacity of an individual as measured by grip force can be, e.g., about 30 psi, about 35 psi, about 40 psi, about 45 psi, about 50 psi, about 55 psi, about 60 psi, about 65 psi, about 70 psi, about 75 psi, about 80 psi, about 85 psi, about 90 psi, about 95 psi, or about 100 psi. In other aspects of this embodiment, a grip capacity of an individual as measured by grip force can be, e.g., at least 30 psi, at least 35 psi, at least 40 psi, at least 45 psi, at least 50 psi, at least 55 psi, at least 60 psi, at least 65 psi, at least 70 psi, at least 75 psi, at least 80 psi, at least 85 psi, at least 90 psi, at least 95 psi, or at least 100 psi. In yet other aspects of this embodiment, a grip capacity of an individual as measured by grip force can be, e.g., at most 30 psi, at most 35 psi, at most 40 psi, at most 45 psi, at most 50 psi, at most 55 psi, at most 60 psi, at most 65 psi, at most 70 psi, at most 75 psi, at most 80 psi, at most 85 psi, at most 90 psi, at most 95 psi, or at most 100 psi. In still other aspects of this embodiment, a grip capacity of an individual as measured by grip force can be from, e.g., about 30 psi to about 40 psi, about 30 psi to about 50 psi, about 30 psi to about 60 psi, about 30 psi to about 70 psi, about 30 psi to about 80 psi, about 30 psi to about 90 psi, about 30 psi to about 100 psi, about 40 psi to about 50 psi, about 40 psi to about 60 psi, about 40 psi to about 70 psi, about 40 psi to about 80 psi, about 40 psi to about 90 psi, about 40 psi to about 100 psi, about 50 psi to about 60 psi, about 50 psi to about 70 psi, about 50 psi to about 80 psi, about 50 psi to about 90 psi, about 50 psi to about 100 psi, about 60 psi to about 70 psi, about 60 psi to about 80 psi, about 60 psi to about 90 psi, about 60 psi to about 100 psi, about 70 psi to about 80 psi, about 70 psi to about 90 psi, about 70 psi to about 100 psi, about 80 psi to about 90 psi, about 80 psi to about 100 psi, or about 90 psi to about 100 psi.

In aspects of this embodiment, a grip capacity of an individual as measured by grip force can be improved by, e.g., about 5 psi, about 10 psi, about 15 psi, about 20 psi, about 25 psi, about 30 psi, about 35 psi, about 40 psi, about 45 psi, or about 50 psi. In other aspects of this embodiment, a grip capacity of an individual as measured by grip force can be improved by, e.g., at least 5 psi, at least 10 psi, at least 15 psi, at least 20 psi, at least 25 psi, at least 30 psi, at least 35 psi, at least 40 psi, at least 45 psi, or at least 50 psi. In yet other aspects of this embodiment, a grip capacity of an individual as measured by grip force can be improved by, e.g., at most 5 psi, at most 10 psi, at most 15 psi, at most 20 psi, at most 25 psi, at most 30 psi, at most 35 psi, at most 40 psi, at most 45 psi, or at most 50 psi. In still other aspects of this embodiment, a grip capacity of an individual as measured by grip force can be improved by, e.g., about 5 psi to about 10 psi, about 5 psi to about 20 psi, about 5 psi to about 30 psi, about 5 psi to about 40 psi, about 5 psi to about 50 psi, about 10 psi to about 20 psi, about 10 psi to about 30 psi, about 10 psi to about 40 psi, about 10 psi to about 50 psi, about 20 psi to about 30 psi, about 20 psi to about 40 psi, about 20 psi to about 50 psi, about 30 psi to about 40 psi, about 30 psi to about 50 psi, or about 40 psi to about 50 psi.

In aspects of this embodiment, a grip capacity of an individual as measured by normal force slippage can be, e.g., about 100 mN, about 125 mN, about 150 mN, about 175 mN, about 200 mN, about 225 mN, about 250 mN, about 275 mN, about 300 mN, about 325 mN, about 350 mN, about 375 mN, about 400 mN, about 425 mN, about 450 mN, about 475 mN, about 500 mN. In other aspects of this embodiment, a grip capacity of an individual as measured by normal force slippage can be, e.g., at least 100 mN, at least 125 mN, at least 150 mN, at least 175 mN, at least 200 mN, at least 225 mN, at least 250 mN, at least 275 mN, at least 300 mN, at least 325 mN, at least 350 mN, at least 375 mN, at least 400 mN, at least 425 mN, at least 450 mN, at least 475 mN, at least 500 mN. In yet other aspects of this embodiment, a grip capacity of an individual as measured by normal force slippage can be, e.g., at most 100 mN, at most 125 mN, at most 150 mN, at most 175 mN, at most 200 mN, at most 225 mN, at most 250 mN, at most 225 mN, at most 250 mN, at most 275 mN, at most 300 mN, at most 325 mN, at most 350 mN, at most 375 mN, at most 400 mN, at most 425 mN, at most 450 mN, at most 475 mN, at most 500 mN. In still other aspects of this embodiment, a grip capacity of an individual as measured by normal force slippage can be from, e.g., about 100 mN to about 125 mN, about 100 mN to about 150 mN, about 100 mN to about 175 mN, about 100 mN to about 200 mN, about 100 mN to about 225 mN, about 100 mN to about 250 mN, about 100 mN to about 275 mN, about 100 mN to about 300 mN, about 100 mN to about 325 mN, about 100 mN to about 350 mN, about 100 mN to about 375 mN, about 100 mN to about 400 mN, about 100 mN to about 425 mN, about 100 mN to about 450 mN, about 100 mN to about 475 mN, about 100 mN to about 500 mN, about 125 mN to about 150 mN, about 125 mN to about 175 mN, about 125 mN to about 200 mN, about 125 mN to about 225 mN, about 125 mN to about 250 mN, about 125 mN to about 275 mN, about 125 mN to about 300 mN, about 125 mN to about 325 mN, about 125 mN to about 350 mN, about 125 mN to about 375 mN, about 125 mN to about 400 mN, about 125 mN to about 425 mN, about 125 mN to about 450 mN, about 125 mN to about 475 mN, about 125 mN to about 500 mN, about 150 mN to about 175 mN, about 150 mN to about 200 mN, about 150 mN to about 225 mN, about 150 mN to about 250 mN, about 150 mN to about 275 mN, about 150 mN to about 300 mN, about 150 mN to about 325 mN, about 150 mN to about 350 mN, about 150 mN to about 375 mN, about 150 mN to about 400 mN, about 150 mN to about 425 mN, about 150 mN to about 450 mN, about 150 mN to about 475 mN, about 150 mN to about 500 mN, about 175 mN to about 200 mN, about 175 mN to about 225 mN, about 175 mN to about 250 mN, about 175 mN to about 275 mN, about 175 mN to about 300 mN, about 175 mN to about 325 mN, about 175 mN to about 350 mN, about 175 mN to about 375 mN, about 175 mN to about 400 mN, about 175 mN to about 425 mN, about 175 mN to about 450 mN, about 175 mN to about 475 mN, about 175 mN to about 500 mN, about 200 mN to about 225 mN, about 200 mN to about 250 mN, about 200 mN to about 275 mN, about 200 mN to about 300 mN, about 200 mN to about 325 mN, about 200 mN to about 350 mN, about 200 mN to about 375 mN, about 200 mN to about 400 mN, about 200 mN to about 425 mN, about 200 mN to about 450 mN, about 200 mN to about 475 mN, about 200 mN to about 500 mN, about 225 mN to about 250 mN, about 225 mN to about 275 mN, about 225 mN to about 300 mN, about 225 mN to about 325 mN, about 225 mN to about 350 mN, about 225 mN to about 375 mN, about 225 mN to about 400 mN, about 225 mN to about 425 mN, about 225 mN to about 450 mN, about 225 mN to about 475 mN, about 225 mN to about 500 mN, about 250 mN to about 275 mN, about 250 mN to about 300 mN, about 250 mN to about 325 mN, about 250 mN to about 350 mN, about 250 mN to about 375 mN, about 250 mN to about 400 mN, about 250 mN to about 425 mN, about 250 mN to about 450 mN, about 250 mN to about 475 mN, about 250 mN to about 500 mN, about 275 mN to about 300 mN, about 275 mN to about 325 mN, about 275 mN to about 350 mN, about 275 mN to about 375 mN, about 275 mN to about 400 mN, about 275 mN to about 425 mN, about 275 mN to about 450 mN, about 275 mN to about 475 mN, about 275 mN to about 500 mN, about 300 mN to about 325 mN, about 300 mN to about 350 mN, about 300 mN to about 375 mN, about 300 mN to about 400 mN, about 300 mN to about 425 mN, about 300 mN to about 450 mN, about 300 mN to about 475 mN, or about 300 mN to about 500 mN.

In aspects of this embodiment, a grip capacity of an individual as measured by normal force slippage can be improved by, e.g., about 10 mN, about 20 mN, about 30 mN, about 40 mN, about 50 mN, about 60 mN, about 70 mN, about 80 mN, about 90 mN, or about 100 mN. In other aspects of this embodiment, a grip capacity of an individual as measured by normal force slippage can be improved by, e.g., at least 10 mN, at least 20 mN, at least 30 mN, at least 40 mN, at least 50 mN, at least 60 mN, at least 70 mN, at least 80 mN, at least 90 mN, or at least 100 mN. In yet other aspects of this embodiment, a grip capacity of an individual as measured by normal force slippage can be improved by, e.g., at most 10 mN, at most 20 mN, at most 30 mN, at most 40 mN, at most 50 mN, at most 60 mN, at most 70 mN, at most 80 mN, at most 90 mN, or at most 100 mN. In still other aspects of this embodiment, a grip capacity of an individual as measured by normal force slippage can be improved by, e.g., about 10 mN to about 20 mN, about 10 mN to about 30 mN, about 10 mN to about 40 mN, about 10 mN to about 50 mN, about 10 mN to about 60 mN, about 10 mN to about 70 mN, about 10 mN to about 80 mN, about 10 mN to about 90 mN, about 10 mN to about 100 mN, about 20 mN to about 30 mN, about 20 mN to about 40 mN, about 20 mN to about 50 mN, about 20 mN to about 60 mN, about 20 mN to about 70 mN, about 20 mN to about 80 mN, about 20 mN to about 90 mN, about 20 mN to about 100 mN, about 30 mN to about 40 mN, about 30 mN to about 50 mN, about 30 mN to about 60 mN, about 30 mN to about 70 mN, about 30 mN to about 80 mN, about 30 mN to about 90 mN, about 30 mN to about 100 mN, about 40 mN to about 50 mN, about 40 mN to about 60 mN, about 40 mN to about 70 mN, about 40 mN to about 80 mN, about 40 mN to about 90 mN, about 40 mN to about 100 mN, about 50 mN to about 60 mN, about 50 mN to about 70 mN, about 50 mN to about 80 mN, about 50 mN to about 90 mN, about 50 mN to about 100 mN, about 60 mN to about 70 mN, about 60 mN to about 80 mN, about 60 mN to about 90 mN, about 60 mN to about 100 mN, about 70 mN to about 80 mN, about 70 mN to about 90 mN, about 70 mN to about 100 mN, about 80 mN to about 90 mN, about 80 mN to about 100 mN, or about 90 mN to about 100 mN.

In aspects of this embodiment, a grip capacity of an individual as measured by the pulling force slippage can be, e.g., about 0.1 N, about 0.5 N, about 1 N, about 1.5 N, about 2 N, about 2.5 N, about 3 N, about 3.5 N, about 4 N, about 4.5 N, or about 5 N. In other aspects of this embodiment, a grip capacity of an individual as measured by the pulling force slippage can be, e.g., at least 0.1 N, at least 0.5 N, at least 1 N, at least 1.5 N, at least 2 mN, at least 2.5 N, at least 3 N, at least 3.5 N, at least 4 N, at least 4.5 N, or at least 5 N. In yet other aspects of this embodiment, a grip capacity of an individual as measured by the pulling force slippage can be, e.g., at most 0.1 N, at most 0.5 N, at most 1 N, at most 1.5 N, at most 2 N, at most 2.5 N, at most 3 N, at most 3.5 N, at most 4 N, at most 4.5 N, or at most 5 N. In still other aspects of this embodiment, a grip capacity of an individual as measured by the pulling force slippage can be from, e.g., about 0.1 N to about 1 N, about 0.1 N to about 2 N, about 0.1 N to about 3 N, about 0.1 N to about 4 N, about 0.1 N to about 5 N, about 0.1 N to about 6 N, about 0.1 N to about 7 N, about 0.1 N to about 8 N, about 0.1 N to about 9 N, about 0.1 N to about 10 N, about 0.5 N to about 1 N, about 0.5 N to about 2 N, about 0.5 N to about 3 N, about 0.5 N to about 4 N, about 0.5 N to about 5 N, about 0.5 N to about 6 N, about 0.5 N to about 7 N, about 0.5 N to about 8 N, about 0.5 N to about 9 N, about 0.5 N to about 10 N, about 1 N to about 2 N, about 1 N to about 3 N, about 1 N to about 4 N, about 1 N to about 5 N, about 1 N to about 6 N, about 1 N to about 7 N, about 1 N to about 8 N, about 1 N to about 9 N, about 1 N to about 10 N, about 2 N to about 3 N, about 2 N to about 4 N, about 2 N to about 5 N, about 2 N to about 6 N, about 2 N to about 7 N, about 2 N to about 8 N, about 2 N to about 9 N, about 2 N to about 10 N, about 3 N to about 4 N, about 3 N to about 5 N, about 3 N to about 6 N, about 3 N to about 7 N, about 3 N to about 8 N, about 3 N to about 9 N, about 3 N to about 10 N, about 4 N to about 5 N, about 4 N to about 6 N, about 4 N to about 7 N, about 4 N to about 8 N, about 4 N to about 9 N, about 4 N to about 10 N, about 5 N to about 6 N, about 5 N to about 7 N, about 5 N to about 8 N, about 5 N to about 9 N, about 5 N to about 10 N, about 6 N to about 7 N, about 6 N to about 8 N, about 6 N to about 9 N, about 6 N to about 10 N, about 7 N to about 8 N, about 7 N to about 9 N, about 7 N to about 10 N, about 8 N to about 9 N, about 8 N to about 10 N, or about 9 N to about 10 N.

In aspects of this embodiment, a grip capacity of an individual as measured by the pulling force slippage can be improved by, e.g., about 0.01 N, about 0.02 N, about 0.03 N, about 0.04 N, about 0.05 N, about 0.06 N, about 0.07 N, about 0.08 N, about 0.09 N, about 0.1 N, about 0.2 N, about 0.3 N, about 0.4 N, about 0.5 N, about 0.6 N, about 0.7 N, about 0.8 N, about 0.9 N, about 1 N, about 2 N, about 3 N, about 4 N, or about 5 N. In other aspects of this embodiment, a grip capacity of an individual as measured by the pulling force slippage can be improved by, e.g., at least 0.01 N, at least 0.02 N, at least 0.03 N, at least 0.04 N, at least 0.05 N, at least 0.06 N, at least 0.07 N, at least 0.08 N, at least 0.09 N, at least 0.1 N, at least 0.2 N, at least 0.3 N, at least 0.4 N, at least 0.5 N, at least 0.6 N, at least 0.7 N, at least 0.8 N, at least 0.9 N, at least 1 N, at least 2 N, at least 3 N, at least 4 N, or at least 5 mN. In yet other aspects of this embodiment, a grip capacity of an individual as measured by the pulling force slippage can be improved by, e.g., at most 0.01 N, at most 0.02 N, at most 0.03 N, at most 0.04 N, at most 0.05 N, at most 0.06 N, at most 0.07 N, at most 0.08 N, at most 0.09 N, at most 0.1 N, at most 0.2 N, at most 0.3 N, at most 0.4 N, at most 0.5 N, at most 0.6 N, at most 0.7 N, at most 0.8 N, at most 0.9 N, at most 1 N, at most 2 N, at most 3 N, at most 4 N, or at most 5 N. In still other aspects of this embodiment, a grip capacity of an individual as measured by the pulling force slippage can be improved by, e.g., about 0.01 N to about 0.05 N, about 0.01 N to about 0.1 N, about 0.01 N to about 0.5 N, about 0.01 N to about 1 N, about 0.01 N to about 2 N, about 0.01 N to about 3 N, about 0.01 N to about 4 N, about 0.01 N to about 5 N, about 0.05 N to about 0.1 N, about 0.05 N to about 0.5 N, about 0.05 N to about 1 N, about 0.05 N to about 2 N, about 0.05 N to about 3 N, about 0.05 N to about 4 N, about 0.05 N to about 5 N, about 0.1 N to about 0.5 N, about 0.1 N to about 1 N, about 0.1 N to about 2 N, about 0.1 N to about 3 N, about 0.1 N to about 4 N, about 0.1 N to about 5 N, about 0.5 N to about 1 N, about 0.5 N to about 2 N, about 0.5 N to about 3 N, about 0.5 N to about 4 N, about 0.5 N to about 5 N, about 1 N to about 2 N, about 1 N to about 3 N, about 1 N to about 4 N, about 1 N to about 5 N, about 2 N to about 3 N, about 2 N to about 4 N, about 2 N to about 5 N, about 3 N to about 4 N, about 3 N to about 5 N, or about 4 N to about 5 N.

In aspects of this embodiment, a grip capacity of an individual as measured by slippage speed can be, e.g., about 1 mm/s, about 5 mm/s, about 10 mm/s, about 15 mm/s, about 20 mm/s, about 25 mm/s, about 30 mm/s, about 35 mm/s, about 40 mm/s, about 45 mm/s, about 50 mm/s, about 55 mm/s, about 60 mm/s, about 65 mm/s, about 70 mm/s, about 75 mm/s, about 80 mm/s, about 85 mm/s, about 90 mm/s, about 95 mm/s, or about 100 mm/s. In other aspects of this embodiment, a grip capacity of an individual as measured by slippage speed can be, e.g., at least 1 mm/s, at least 5 mm/s, at least 10 mm/s, at least 15 mm/s, at least 20 mm/s, at least 25 mm/s, at least 30 mm/s, at least 35 mm/s, at least 40 mm/s, at least 45 mm/s, at least 50 mm/s, at least 55 mm/s, at least 60 mm/s, at least 65 mm/s, at least 70 mm/s, at least 75 mm/s, at least 80 mm/s, at least 85 mm/s, at least 90 mm/s, at least 95 mm/s, or at least 100 mm/s. In yet other aspects of this embodiment, a grip capacity of an individual as measured by slippage speed can be, e.g., at most 1 mm/s, at most 5 mm/s, at most 10 mm/s, at most 15 mm/s, at most 20 mm/s, at most 25 mm/s, at most 30 mm/s, at most 35 mm/s, at most 40 mm/s, at most 45 mm/s, at most 50 mm/s, at most 55 mm/s, at most 60 mm/s, at most 65 mm/s, at most 70 mm/s, at most 75 mm/s, at most 80 mm/s, at most 85 mm/s, at most 90 mm/s, at most 95 mm/s, or at most 100 mm/s. In still other aspects of this embodiment, a grip capacity of an individual as measured by slippage speed can be from, e.g., about 10 mm/s to about 20 mm/s, about 10 mm/s to about 30 mm/s, about 10 mm/s to about 40 mm/s, about 10 mm/s to about 50 mm/s, about 10 mm/s to about 60 mm/s, about 10 mm/s to about 70 mm/s, about 10 mm/s to about 80 mm/s, about 10 mm/s to about 90 mm/s, about 10 mm/s to about 100 mm/s, about 20 mm/s to about 30 mm/s, about 20 mm/s to about 40 mm/s, about 20 mm/s to about 50 mm/s, about 20 mm/s to about 60 mm/s, about 20 mm/s to about 70 mm/s, about 20 mm/s to about 80 mm/s, about 20 mm/s to about 90 mm/s, about 20 mm/s to about 100 mm/s, about 30 mm/s to about 40 mm/s, about 30 mm/s to about 50 mm/s, about 30 mm/s to about 60 mm/s, about 30 mm/s to about 70 mm/s, about 30 mm/s to about 80 mm/s, about 30 mm/s to about 90 mm/s, about 30 mm/s to about 100 mm/s, about 40 mm/s to about 50 mm/s, about 40 mm/s to about 60 mm/s, about 40 mm/s to about 70 mm/s, about 40 mm/s to about 80 mm/s, about 40 mm/s to about 90 mm/s, about 40 mm/s to about 100 mm/s, about 50 mm/s to about 60 mm/s, about 50 mm/s to about 70 mm/s, about 50 mm/s to about 80 mm/s, about 50 mm/s to about 90 mm/s, about 50 mm/s to about 100 mm/s, about 60 mm/s to about 70 mm/s, about 60 mm/s to about 80 mm/s, about 60 mm/s to about 90 mm/s, about 60 mm/s to about 100 mm/s, about 70 mm/s to about 80 mm/s, about 70 mm/s to about 90 mm/s, about 70 mm/s to about 100 mm/s, about 80 mm/s to about 90 mm/s, about 80 mm/s to about 100 mm/s, or about 90 mm/s to about 100 mm/s.

In aspects of this embodiment, a grip capacity of an individual as measured by grip friction speed can be improved by, e.g., about 1 mm/s, about 5 mm/s, about 10 mm/s, about 15 mm/s, about 20 mm/s, about 25 mm/s, about 30 mm/s, about 35 mm/s, about 40 mm/s, about 45 mm/s, or about 50 mm/s. In other aspects of this embodiment, a grip capacity of an individual as measured by grip friction speed can be improved by, e.g., at least 1 mm/s, at least 5 mm/s, at least 10 mm/s, at least 15 mm/s, at least 20 mm/s, at least 25 mm/s, at least 30 mm/s, at least 35 mm/s, at least 40 mm/s, at least 45 mm/s, or at least 50 mm/s. In yet other aspects of this embodiment, a grip capacity of an individual as measured by grip friction speed can be improved by, e.g., at most 1 mm/s, at most 5 mm/s, at most 10 mm/s, at most 15 mm/s, at most 20 mm/s, at most 25 mm/s, at most 30 mm/s, at most 35 mm/s, at most 40 mm/s, at most 45 mm/s, or at most 50 mm/s. In still other aspects of this embodiment, a grip capacity of an individual as measured by grip friction speed can be improved by, e.g., about 1 mm/s to about 5 mm/s, about 1 mm/s to about 10 mm/s, about 1 mm/s to about 20 mm/s, about 1 mm/s to about 30 mm/s, about 1 mm/s to about 40 mm/s, about 1 mm/s to about 50 mm/s, about 5 mm/s to about 10 mm/s, about 5 mm/s to about 20 mm/s, about 5 mm/s to about 30 mm/s, about 5 mm/s to about 40 mm/s, about 5 mm/s to about 50 mm/s, about 10 mm/s to about 20 mm/s, about 10 mm/s to about 30 mm/s, about 10 mm/s to about 40 mm/s, about 10 mm/s to about 50 mm/s, about 20 mm/s to about 30 mm/s, about 20 mm/s to about 40 mm/s, about 20 mm/s to about 50 mm/s, about 30 mm/s to about 40 mm/s, about 30 mm/s to about 50 mm/s, or about 40 mm/s to about 50 mm/s.

Aspects of the present specification disclose a method or use to protect a skin region of an individual. Protecting a skin region is the ability to reducing, ameliorating, eliminating or preventing an irritation, a pain or other symptom that causes discomfort or displeasure to an individual. In one embodiment, protecting a skin region is assessed by the ability of an individual to competently, proficiently and/or safely execute an activity being performed. The actual symptoms associated with an irritation, a pain or other symptom that causes discomfort or displeasure to an individual are well known and can be determined by a person of ordinary skill in the art by taking into account factors, including, without limitation, the location of the irritation, pain or other symptom that causes discomfort or displeasure to an individual, the cause of the irritation, pain or other symptom that causes discomfort or displeasure to an individual, the severity of the irritation, pain or other symptom that causes discomfort or displeasure to an individual, and/or the skin region affected by the irritation, pain or other symptom that causes discomfort or displeasure to an individual. Those of skill in the art will know the appropriate symptoms or indicators associated with a specific type of irritation, pain or other symptom that causes discomfort or displeasure to an individual and will know how to determine if an individual is a candidate for treatment as disclosed herein.

A grip enhancing composition, method or use disclosed herein reduced unwanted side effects associated with block or powdered chalk and alcohol-based liquid chalk compositions. In one embodiment, a grip enhancing composition, method or use disclosed herein reduces the amount of grip enhancer that becomes airborne as dust particles due to application or use. In another embodiment, a grip enhancing composition, method or use disclosed herein reduces eye irritation, respiratory discomfort, or both. In yet another embodiment, a grip enhancing composition, method or use disclosed herein reduces chapped skin, dry skin, cracked skin, irritated skin, itchiness, skin tightness, rough skin, red plaques, discomfort, pain, or any combination thereof. In another embodiment, a grip enhancing composition, method or use disclosed herein reduces the flammability of the product. In another embodiment, a grip enhancing composition, method or use disclosed herein is not flammable. In another embodiment, a grip enhancing composition, method or use disclosed herein reduces unwanted deposition of grip enhancer on fabric, hair, or skin on areas that the product is not directly applied.

Aspects of the specification can also be described as follows:
1. A grip enhancing composition comprising, consisting essentially of, or consisting of one or more grip enhancers and one or more hydrophilic clays, wherein the grip enhancing composition does not include an alcohol.
2. The grip enhancing composition according to embodiment 1, wherein the one or more hydrophilic clays has thixotrophic properties.
3. The grip enhancing composition according to embodiment 1 or embodiment 2, wherein the one or more hydrophilic clays comprises, consists essentially of, or consists of one or more bentonite clays, one or more hectorite clays, one or more kaolinite clays, one or more silicate clays, or any combination thereof.
4. The grip enhancing composition according to embodiment 3, wherein the one or more bentonite clays include a potassium bentonite clay, a sodium bentonite clay, a calcium bentonite clay, an aluminum bentonite clay, or any combination thereof.
5. The grip enhancing composition according to embodiment 3, wherein the one or more silicate clays include a lithium magnesium sodium silicate clay, a sodium magnesium fluorosilicate clay, or any combination thereof.
6. The grip enhancing composition according to any one of embodiments 1-5, wherein the one or more hydrophilic clays in an amount of 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%. 9%, or 10% by weight of the composition; at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, or at least 10% by weight of the composition; at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, or at most 10% by weight of the composition, or any combination thereof; or about 0.01% to about 0.05%, 0.01% to about 0.075%, about 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10% or about 9% to about 10% by weight of the composition.
7. The grip enhancing composition according to any one of embodiments 1-6, wherein the grip enhancing composition does not comprise, consist essentially of, or consist of one or more polysaccharides.
8. The grip enhancing composition according to any one of embodiments 1-6, further comprising, consisting essentially of, or consisting of one or more polysaccharides.
9. A grip enhancing composition comprising, consisting essentially of, or consisting of one or more grip enhancers and one or more polysaccharides, wherein the grip enhancing composition does not include an alcohol.
10. The grip enhancing composition according to embodiment 8 or embodiment 9, wherein the one or more polysaccharides comprise, consist essentially of, or consist of cellulose, one or more cellulose ether derivatives, one or more cellulose ester derivatives, any salt thereof, or any combination thereof.
11. The grip enhancing composition according to embodiment 10, wherein the one or more cellulose ether derivatives include ethyl cellulose, methyl cellulose, ethylmethyl cellulose, hydroxyethyl cellulose, hydroxypropyl cellulose, hydroxyethylmethyl cellulose, hydroxypropylmethyl cellulose, ethyl hydroxyethyl cellulose, carboxymethyl cellulose, or any salt thereof.
12. The grip enhancing composition according to embodiment 10, wherein the one or more cellulose ester derivatives include cellulose acetate, cellulose acetate phthalate, cellulose acetate butyrate, cellulose acetate trimelitate, hydroxupropylmethyl cellulose phthalate, or any salt thereof.
13. The grip enhancing composition according to any one of embodiments 8-12, wherein the one or more polysaccharides is in an amount of 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%. 9%, or 10% by weight of the composition; at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, or at least 10% by weight of the composition; at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, or at most 10% by weight of the composition, or any combination thereof; or about 0.01% to about 0.05%, 0.01% to about 0.075%, about 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10% or about 9% to about 10% by weight of the composition.
14. The grip enhancing composition according to any one of embodiments 8-13, further comprising, consisting essentially of, or consisting of one or more hydrophilic clays.
15. The grip enhancing composition according to any one of embodiments 8-13, wherein the grip enhancing composition does not comprise, consist essentially of, or consist of one or more hydrophilic clays.
16. The grip enhancing composition according to any one of embodiments 1-15, wherein the one or more grip enhancers comprise, consist essentially of, or consist of one or more chalks, one or more talc, one or more resins, one or more rosins, or any combination thereof.
17. The grip enhancing composition according to embodiment 16, wherein the one or more chalks include magnesium carbonate, calcium carbonate, or any combination thereof.
18. The grip enhancing composition according to embodiment 16 or embodiment 17, wherein the one or more talcs include magnesium silicate, calcium silicate, or any combination thereof.
19. The grip enhancing composition according to any one of embodiments 16-18, wherein the one or more grip enhancers in an amount of 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%. 9%, 10%, 15%, 20%, 25%, 30%, 35%, 40%, 45%, 50%, 55%, 60%, 65%, 70%, 75%, 80%, 85%, 90%, or 95% by weight of the composition; at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% by weight of the composition; at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, at most 10%, at most 15%, at most 20%, at most 25%, at most 30%, at most 35%, at most 40%, 40%, at most 45%, at most 50%, at most 55%, at most 60%, at most 65%, at most 70%, at most 75%, at most 80%, at most 85%, at most 90%, or at most 95% by weight of the composition, or any combination thereof; about 0.01% to about 0.05%, about 0.01% to about 0.075%, about 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 15%, about 0.1% to about 20%, about 0.1% to about 25%, about 0.1% to about 30%, about 0.1% to about 35%, about 0.1% to about 40%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.25% to about 15%, about 0.25% to about 20%, about 0.25% to about 25%, about 0.25% to about 30%, about 0.25% to about 35%, about 0.25% to about 40%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.5% to about 15%, about 0.5% to about 20%, about 0.5% to about 25%, about 0.5% to about 30%, about 0.5% to about 35%, about 0.5% to about 40%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 0.75% to about 15%, about 0.75% to about 20%, about 0.75% to about 25%, about 0.75% to about 30%, about 0.75% to about 35%, about 0.75% to about 40%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 15%, about 1% to about 20%, about 1% to about 25%, about 1% to about 30%, about 1% to about 35%, about 1% to about 40%, about 2% to about 3%, about 2% to about 4%, about 2% to about 5%, about 2% to about 6%, about 2% to about 7%, about 2% to about 8%, about 2% to about 9%, about 2% to about 10%, about 2% to about 15%, about 2% to about 20%, about 2% to about 25%, about 2% to about 30%, about 2% to about 35%, about 2% to about 40%, about 3% to about 4%, about 3% to about 5%, about 3% to about 6%, about 3% to about 7%, about 3% to about 8%, about 3% to about 9%, about 3% to about 10%, about 3% to about 15%, about 3% to about 20%, about 3% to about 25%, about 3% to about 30%, about 3% to about 35%, about 3% to about 40%, about 4% to about 5%, about 4% to about 6%, about 4% to about 7%, about 4% to about 8%, about 4% to about 9%, about 4% to about 10%, about 4% to about 15%, about 4% to about 20%, about 4% to about 25%, about 4% to about 30%, about 4% to about 35%, about 4% to about 40%, about 5% to about 6%, about 5% to about 7%, about 5% to about 8%, about 5% to about 9%, about 5% to about 10%, about 5% to about 15%, about 5% to about 20%, about 5% to about 25%, about 5% to about 30%, about 5% to about 35%, about 5% to about 40%, about 6% to about 7%, about 6% to about 8%, about 6% to about 9%, about 6% to about 10%, about 6% to about 15%, about 6% to about 20%, about 6% to about 25%, about 6% to about 30%, about 6% to about 35%, about 6% to about 40%, about 7% to about 8%, about 7% to about 9%, about 7% to about 10%, about 7% to about 15%, about 7% to about 20%, about 7% to about 25%, about 7% to about 30%, about 7% to about 35%, about 7% to about 40%, about 8% to about 9%, about 8% to about 10%, about 8% to about 15%, about 8% to about 20%, about 8% to about 25%, about 8% to about 30%, about 8% to about 35%, about 8% to about 40%, about 9% to about 10%, about 9% to about 15%, about 9% to about 20%, about 9% to about 25%, about 9% to about 30%, about 9% to about 35%, about 9% to about 40%, about 10% to about 15%, about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, or about 35% to about 40% by weight of the composition; or about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 20% to about 30%, about 20% to about 40%, about 20% to about 50%, about 20% to about 60%, about 20% to about 70%, about 20% to about 80%, about 20% to about 90%, about 20% to about 95%, about 30% to about 40%, about 30% to about 50%, about 30% to about 60%, about 30% to about 70%, about 30% to about 80%, about 30% to about 90%, about 30% to about 95%, about 40% to about 50%, about 40% to about 60%, about 40% to about 70%, about 40% to about 80%, about 40% to about 90%, about 40% to about 95%, about 50% to about 60%, about 50% to about 70%, about 50% to about 80%, about 50% to about 90%, about 50% to about 95%, about 60% to about 70%, about 60% to about 80%, about 60% to about 90%, about 60% to about 95%, about 70% to about 80%, about 70% to about 90%, about 70% to about 95%, about 80% to about 90%, about 80% to about 95%, or about 90% to about 95% by weight of the composition.
20. The grip enhancing composition according to any one of embodiments 1-19, further comprising, consisting essentially of, or consisting of a silica.
21. The grip enhancing composition according to embodiment 20, wherein the silica is in an amount of 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%. 9%, 10%, 15%, 20%, 25%, 30%, 35%, or 40% by weight of the composition or at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, least 10%, least 15%, least 20%, least 25%, least 30%, least 35%, or least 40% by weight of the composition or at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, most 10%, most 15%, most 20%, most 25%, most 30%, most 35%, or most 40% by weight of the composition, or any combination thereof; or 0.01% to about 0.05%, 0.01% to about 0.075%, 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 15%, about 0.1% to about 20%, about 0.1% to about 25%, about 0.1% to about 30%, about 0.1% to about 35%, about 0.1% to about 40%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.25% to about 15%, about 0.25% to about 20%, about 0.25% to about 25%, about 0.25% to about 30%, about 0.25% to about 35%, about 0.25% to about 40%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.5% to about 15%, about 0.5% to about 20%, about 0.5% to about 25%, about 0.5% to about 30%, about 0.5% to about 35%, about 0.5% to about 40%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 0.75% to about 15%, about 0.75% to about 20%, about 0.75% to about 25%, about 0.75% to about 30%, about 0.75% to about 35%, about 0.75% to about 40%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 15%, about 1% to about 20%, about 1% to about 25%, about 1% to about 30%, about 1% to about 35%, about 1% to about 40%, about 2% to about 3%, about 2% to about 4%, about 2% to about 5%, about 2% to about 6%, about 2% to about 7%, about 2% to about 8%, about 2% to about 9%, about 2% to about 10%, about 2% to about 15%, about 2% to about 20%, about 2% to about 25%, about 2% to about 30%, about 2% to about 35%, about 2% to about 40%, about 3% to about 4%, about 3% to about 5%, about 3% to about 6%, about 3% to about 7%, about 3% to about 8%, about 3% to about 9%, about 3% to about 10%, about 3% to about 15%, about 3% to about 20%, about 3% to about 25%, about 3% to about 30%, about 3% to about 35%, about 3% to about 40%, about 4% to about 5%, about 4% to about 6%, about 4% to about 7%, about 4% to about 8%, about 4% to about 9%, about 4% to about 10%, about 4% to about 15%, about 4% to about 20%, about 4% to about 25%, about 4% to about 30%, about 4% to about 35%, about 4% to about 40%, about 5% to about 6%, about 5% to about 7%, about 5% to about 8%, about 5% to about 9%, about 5% to about 10%, about 5% to about 15%, about 5% to about 20%, about 5% to about 25%, about 5% to about 30%, about 5% to about 35%, about 5% to about 40%, about 6% to about 7%, about 6% to about 8%, about 6% to about 9%, about 6% to about 10%, about 6% to about 15%, about 6% to about 20%, about 6% to about 25%, about 6% to about 30%, about 6% to about 35%, about 6% to about 40%, about 7% to about 8%, about 7% to about 9%, about 7% to about 10%, about 7% to about 15%, about 7% to about 20%, about 7% to about 25%, about 7% to about 30%, about 7% to about 35%, about 7% to about 40%, about 8% to about 9%, about 8% to about 10%, about 8% to about 15%, about 8% to about 20%, about 8% to about 25%, about 8% to about 30%, about 8% to about 35%, about 8% to about 40%, about 9% to about 10%, about 9% to about 15%, about 9% to about 20%, about 9% to about 25%, about 9% to about 30%, about 9% to about 35%, about 9% to about 40%, about 10% to about 15%, about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, or about 35% to about 40% by weight of the composition.
22. The grip enhancing composition according to any one of embodiments 1-21, further comprising, consisting essentially of, or consisting of hypochlorous acid or free available chlorine.
23. The grip enhancing composition according to embodiment 22, wherein the hypochlorous acid or free available chlorine is in an amount of 0.00005%, 0.00006%, 0.00007%, 0.000075%, 0.00008%, 0.00009%, 0.0001%, 0.0005%, 0.001%, 0.0015%, 0.002%, 0.0025%, 0.003%, 0.0035%, 0.004%, 0.0045%, 0.005%, 0.0055%, 0.006%, 0.007%, 0.0075%, 0.008%, 0.009%, 0.01%, 0.02%, 0.03%, 0.04% or 0.05% by weight of the composition or at least 0.00005%, at least 0.00006%, at least 0.00007%, at least 0.000075%, at least 0.00008%, at least 0.00009%, at least 0.0001%, at least 0.0005%, at least 0.001%, at least 0.0015%, at least 0.002%, at least 0.0025%, at least 0.003%, at least 0.0035%, at least 0.004%, at least 0.0045%, at least 0.005%, at least 0.0055%, at least 0.006%, at least 0.007%, at least 0.0075%, at least 0.008%, at least 0.009%, at least 0.01%, at least 0.02%, at least 0.03%, at least 0.04% or at least 0.05% by weight of the composition or at most 0.00005%, at most 0.00006%, at most 0.00007%, at most 0.000075%, at most 0.00008%, at most 0.00009%, at most 0.0001%, at most 0.0005%, at most 0.001%, at most 0.0015%, at most 0.002%, at most 0.0025%, at most 0.003%, at most 0.0035%, at most 0.004%, at most 0.0045%, at most 0.005%, at most 0.0055%, at most 0.006%, at most 0.007%, at most 0.0075%, at most 0.008%, at most 0.009%, at most 0.01%, at most 0.02%, at most 0.03%, at most 0.04% or at most 0.05% by weight of the composition, or any combination thereof; or about 0.00075% to about 0.002%, about 0.00075% to about 0.003%, about 0.00075% to about 0.004%, about 0.00075% to about 0.005%, about 0.00075% to about 0.006%, about 0.00075% to about 0.007%, about 0.00075% to about 0.008%, about 0.00075% to about 0.009%, about 0.00075% to about 0.01%, about 0.001% to about 0.002%, about 0.001% to about 0.003%, about 0.001% to about 0.004%, about 0.001% to about 0.005%, about 0.001% to about 0.006%, about 0.001% to about 0.007%, about 0.001% to about 0.008%, about 0.001% to about 0.009%, about 0.001% to about 0.01%, about 0.002% to about 0.003%, about 0.002% to about 0.004%, about 0.002% to about 0.005%, about 0.002% to about 0.006%, about 0.002% to about 0.007%, about 0.002% to about 0.008%, about 0.002% to about 0.009%, about 0.002% to about 0.01% by weight of the composition
24. The grip enhancing composition according to embodiment 22 or 23, wherein the hypochlorous acid or free available chlorine is in an amount of 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm or 400 ppm or at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm or at least 400 ppm or at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm or at most 400 ppm, or any combination thereof; or about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm or about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.
25. The grip enhancing composition according to any one of embodiments 1-22, further comprising, consisting essentially of, or consisting of an ascorbic acid.
26. The grip enhancing composition according to embodiment 25, wherein the ascorbic acid is in an amount of 0.00005%, 0.00006%, 0.00007%, 0.000075%, 0.00008%, 0.00009%, 0.0001%, 0.0005%, 0.001%, 0.0015%, 0.002%, 0.0025%, 0.003%, 0.0035%, 0.004%, 0.0045%, 0.005%, 0.0055%, 0.006%, 0.007%, 0.0075%, 0.008%, 0.009%, 0.01%, 0.02%, 0.03%, 0.04% or 0.05% by weight of the composition or at least 0.00005%, at least 0.00006%, at least 0.00007%, at least 0.000075%, at least 0.00008%, at least 0.00009%, at least 0.0001%, at least 0.0005%, at least 0.001%, at least 0.0015%, at least 0.002%, at least 0.0025%, at least 0.003%, at least 0.0035%, at least 0.004%, at least 0.0045%, at least 0.005%, at least 0.0055%, at least 0.006%, at least 0.007%, at least 0.0075%, at least 0.008%, at least 0.009%, at least 0.01%, at least 0.02%, at least 0.03%, at least 0.04% or at least 0.05% by weight of the composition or at most 0.00005%, at most 0.00006%, at most 0.00007%, at most 0.000075%, at most 0.00008%, at most 0.00009%, at most 0.0001%, at most 0.0005%, at most 0.001%, at most 0.0015%, at most 0.002%, at most 0.0025%, at most 0.003%, at most 0.0035%, at most 0.004%, at most 0.0045%, at most 0.005%, at most 0.0055%, at most 0.006%, at most 0.007%, at most 0.0075%, at most 0.008%, at most 0.009%, at most 0.01%, at most 0.02%, at most 0.03%, at most 0.04% or at most 0.05% by weight of the composition, or any combination thereof; or about 0.00075% to about 0.002%, about 0.00075% to about 0.003%, about 0.00075% to about 0.004%, about 0.00075% to about 0.005%, about 0.00075% to about 0.006%, about 0.00075% to about 0.007%, about 0.00075% to about 0.008%, about 0.00075% to about 0.009%, about 0.00075% to about 0.01%, about 0.001% to about 0.002%, about 0.001% to about 0.003%, about 0.001% to about 0.004%, about 0.001% to about 0.005%, about 0.001% to about 0.006%, about 0.001% to about 0.007%, about 0.001% to about 0.008%, about 0.001% to about 0.009%, about 0.001% to about 0.01%, about 0.002% to about 0.003%, about 0.002% to about 0.004%, about 0.002% to about 0.005%, about 0.002% to about 0.006%, about 0.002% to about 0.007%, about 0.002% to about 0.008%, about 0.002% to about 0.009%, about 0.002% to about 0.01% by weight of the composition
27. The grip enhancing composition according to embodiment 25 or 26, wherein the ascorbic acid is in an amount of 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm or 400 ppm or at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm or at least 400 ppm or at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm or at most 400 ppm, or any combination thereof; or about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm or about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.
28. The grip enhancing composition according to any one of embodiments 1-27, further comprising, consisting essentially of, or consisting of one or more antimicrobial agents.
29. The grip enhancing composition according to embodiment 28, wherein the one or more antimicrobial agents comprise, consist essentially of, or consist of one or more antibacterial agents, one or more antifungal agents, one or more antiviral agents, one or more antiparasitic agents, or any combination thereof.
30. The grip enhancing composition according to embodiment 28 or embodiment 29, wherein the one or more antimicrobial agents is a microbicidal agent or a biostatic agent.
31. The grip enhancing composition according to any one of embodiments 28-30, wherein the one or more antimicrobial agents is a disinfectant, an antiseptic agent or an antibiotic agent.
32. The grip enhancing composition according to any one of embodiments 1-31, further comprising, consisting essentially of, or consisting of one or more fragrances.
33. The grip enhancing composition according to embodiment 32, wherein the one or more fragrances comprise, consist essentially of, or consist of an aldehyde-based fragrance, an amine-based fragrance, an aromatic-based fragrance, an ester-based fragrance, a ketone-based fragrance, a lactone-based fragrance, a thiol-based fragrance, a terpene-based fragrance, or any combination thereof.
34. The grip enhancing composition according to any one of embodiments 1-33, further comprising, consisting essentially of, or consisting of one or more carriers.
35. The grip enhancing composition according to embodiment 34, wherein the one or more carriers include an aqueous carrier, a semi-solid carrier, a solid carrier, or any combination thereof.
36. The grip enhancing composition according to embodiment 34 or 35, wherein the one or more carriers include water, a vegetable oil, a mineral oil, an ester oil, an ether, an alcohol, a fatty alcohol, an isoparaffin, a hydrocarbon oil, a polyol, a wax, or any combination thereof.
37. The grip enhancing composition according to any one of embodiments 34-36, wherein the one or more carriers are in an amount of at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% or at least 99% by weight of the composition or at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 96%, at most 97%, at most 98% or at most 99% by weight of the composition or about 25% to about 50%, about 25% to about 75%, about 25% to about 90%, about 25% to about 95%, about 25% to about 96%, about 25% to about 97%, about 25% to about 98%, about 25% to about 99%, about 50% to about 75%, about 50% to about 90%, about 50% to about 95%, about 50% to about 96%, about 50% to about 97%, about 50% to about 98%, about 50% to about 99%, about 75% to about 80%, about 75% to about 85%, about 75% to about 90%, about 75% to about 95%, about 75% to about 96%, about 75% to about 97%, about 75% to about 98%, about 75% to about 99%, about 80% to about 85%, about 80% to about 90%, about 80% to about 95%, about 80% to about 96%, about 80% to about 97%, about 80% to about 98%, about 80% to about 99%, about 85% to about 90%, about 85% to about 95%, about 85% to about 96%, about 85% to about 97%, about 85% to about 98%, about 85% to about 99%, about 90% to about 95%, about 90% to about 96%, about 90% to about 97%, about 90% to about 98%, about 90% to about 99%, or about 95% to about 99%, by weight of the composition.
38. The grip enhancing composition according to any one of embodiments 1-37, further comprising, consisting essentially of, or consisting of one or more additional ingredients.
39. The grip enhancing composition according to embodiment 38, wherein the one or more additional ingredients include a humectant, a preservative, an emulsifier, a chelating agent, an astringent, a shine control agent, a colorant agent, a soothing agent, a calming agent, a supplement, an herbal agent, a naturopathic agent, or any combination thereof.
40. The grip enhancing composition according to any one of embodiments 1-39, having a pH of at least pH 5.0, at least pH 5.5, at least pH 6.0, at least pH 6.5, at least pH 7.0, at least pH 7.5, at least pH 8.0, at least pH 8.5, at least pH 9.0, at least pH 9.5, at least pH 10.0, at least pH 10.5, at least pH 11.0, at least pH 11.5, or at least pH 12.0; at most pH 5.0, at most pH 5.5, at most pH 6.0, at most pH 6.5, at most pH 7.0, at most pH 7.5, at most pH 8.0, at most pH 8.5, at most pH 9.0, at most pH 9.5, at most pH 10.0, at most pH 10.5, at most pH 11.0, at most pH 11.5, or at most pH 12.0; or about pH 5.0 to about pH 8.0, about pH 5.0 to about pH 8.5, about pH 5.0 to about pH 9.0, about pH 5.0 to about pH 9.5, about pH 5.0 to about pH 10.0, about pH 5.0 to about pH 10.5, about pH 6.0 to about pH 8.0, about pH 6.0 to about pH 8.5, about pH 6.0 to about pH 9.0, about pH 6.0 to about pH 9.5, about pH 6.0 to about pH 10.0, about pH 6.0 to about pH 10.5, about pH 7.0 to about pH 8.0, about pH 7.0 to about pH 8.5, about pH 7.0 to about pH 9.0, about pH 7.0 to about pH 9.5, about pH 7.0 to about pH 10.0, about pH 7.0 to about pH 10.5, about pH 8.0 to about pH 9.0, about pH 8.0 to about pH 9.5, about pH 8.0 to about pH 10.0, about pH 8.0 to about pH 10.5, about pH 9.0 to about pH 9.5, about pH 9.0 to about pH 10.0, about pH 9.0 to about pH 10.5, about pH 9.0 to about pH 11, about pH 9.0 to about pH 11.5, about pH 9.0 to about pH 12, about pH 9.5 to about pH 10.0, about pH 9.5 to about pH 10.5, about pH 9.5 to about pH 11, about pH 9.5 to about pH 11.5, about pH 9.5 to about pH 12, about pH 10.0 to about pH 10.5, about pH 10.0 to about pH 11, about pH 10.0 to about pH 11.5, about pH 10.0 to about pH 12, about pH 10.5 to about pH 11, about pH 10.5 to about pH 11.5, about pH 10.5 to about pH 12, about pH 11.0 to about pH 11.5, about pH 11.0 to about pH 12, or pH 11.5 to about pH 12.
41. The grip enhancing composition according to any one of embodiments 1-40, further comprising electrolytic alkaline water.
42. The grip enhancing composition according to any one of embodiments 1-41, having a stability of about 1 week, about 2 weeks, 3 weeks, 4 weeks, 5 weeks, 6, weeks, 7 weeks, 8 weeks, 9 weeks, 10 weeks, 11 weeks, 12 weeks, 13 weeks, 14 weeks or 15 weeks or at least 1 week, at least 2 weeks, at least 3 weeks, at least 4 weeks, at least 5 weeks, at least 6, weeks, at least 7 weeks, at least 8 weeks, at least 9 weeks, at least 10 weeks, at least 11 weeks, at least 12 weeks, at least 13 weeks, at least 14 weeks or at least 15 weeks or at most 1 week, at most 2 weeks, at most 3 weeks, at most 4 weeks, at most 5 weeks, at most 6, weeks, at most 7 weeks, at most 8 weeks, at most 9 weeks, at most 10 weeks, at most 11 weeks, at most 12 weeks, at most 13 weeks, at most 14 weeks or at most 15 weeks or about 1 week to about 2 weeks, about 1 week to about 3 weeks, about 1 week to about 4 weeks, about 1 week to about 5 weeks, about 1 week to about 6 weeks, about 1 week to about 7 weeks, about 1 week to about 8 weeks, about 1 week to about 9 weeks, about 1 week to about 10 weeks, about 1 week to about 11 weeks, about 1 week to about 12 weeks, about 1 week to about 13 weeks, about 1 week to about 14 weeks, about 1 week to about 15 weeks, about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 2 weeks to about 11 weeks, about 2 weeks to about 12 weeks, about 2 weeks to about 13 weeks, about 2 weeks to about 14 weeks, about 2 weeks to about 15 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 3 weeks to about 11 weeks, about 3 weeks to about 12 weeks, about 3 weeks to about 13 weeks, about 3 weeks to about 14 weeks, about 3 weeks to about 15 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 4 weeks to about 11 weeks, about 4 weeks to about 12 weeks, about 4 weeks to about 13 weeks, about 4 weeks to about 14 weeks, about 4 weeks to about 15 weeks, about 5 weeks to about 6 weeks, about 5 weeks to about 7 weeks, about 5 weeks to about 8 weeks, about 5 weeks to about 9 weeks, about 5 weeks to about 10 weeks, about 5 weeks to about 11 weeks, about 5 weeks to about 12 weeks, about 5 weeks to about 13 weeks, about 5 weeks to about 14 weeks, about 5 weeks to about 15 weeks, about 6 weeks to about 7 weeks, about 6 weeks to about 8 weeks, about 6 weeks to about 9 weeks, about 6 weeks to about 10 weeks, about 6 weeks to about 11 weeks, about 6 weeks to about 12 weeks, about 6 weeks to about 13 weeks, about 6 weeks to about 14 weeks, about 6 weeks to about 15 weeks, about 7 weeks to about 8 weeks, about 7 weeks to about 9 weeks, about 7 weeks to about 10 weeks, about 7 weeks to about 11 weeks, about 7 weeks to about 12 weeks, about 7 weeks to about 13 weeks, about 7 weeks to about 14 weeks, about 7 weeks to about 15 weeks, about 8 weeks to about 9 weeks, about 8 weeks to about 10 weeks, about 8 weeks to about 11 weeks, about 8 weeks to about 12 weeks, about 8 weeks to about 13 weeks, about 8 weeks to about 14 weeks, about 8 weeks to about 15 weeks, about 9 weeks to about 10 weeks, about 9 weeks to about 11 weeks, about 9 weeks to about 12 weeks, about 9 weeks to about 13 weeks, about 9 weeks to about 14 weeks, about 9 weeks to about 15 weeks, about 10 weeks to about 11 weeks, about 10 weeks to about 12 weeks, about 10 weeks to about 13 weeks, about 10 weeks to about 14 weeks, about 10 weeks to about 15 weeks, about 11 weeks to about 12 weeks, about 11 weeks to about 13 weeks, about 11 weeks to about 14 weeks, about 11 weeks to about 15 weeks, about 12 weeks to about 13 weeks, about 12 weeks to about 14 weeks, about 12 weeks to about 15 weeks, about 13 weeks to about 14 weeks, about 13 weeks to about 15 weeks, or about 14 weeks to about 15 weeks.
43. The grip enhancing composition according to any one of embodiments 1-41, having a stability of about 1 month to about 2 months, about 1 month to about 3 months, about 1 month to about 4 months, about 1 month to about 5 months, about 1 month to about 6 months, about 1 month to about 7 months, about 1 month to about 8 months, about 1 month to about 9 months, about 1 month to about 10 months, about 1 month to about 11 months, about 1 month to about 12 months, about 1 month to about 13 months, about 1 month to about 14 months, about 1 month to about 15 months, about 1 month to about 16 months, about 1 month to about 17 months, about 1 month to about 18 months, about 2 months to about 3 months, about 2 months to about 4 months, about 2 months to about 5 months, about 2 months to about 6 months, about 2 months to about 7 months, about 2 months to about 8 months, about 2 months to about 9 months, about 2 months to about 10 months, about 2 months to about 11 months, about 2 months to about 12 months, about 2 months to about 13 months, about 2 months to about 14 months, about 2 months to about 15 months, about 2 months to about 16 months, about 2 months to about 17 months, about 2 months to about 18 months, about 3 months to about 4 months, about 3 months to about 5 months, about 3 months to about 6 months, about 3 months to about 7 months, about 3 months to about 8 months, about 3 months to about 9 months, about 3 months to about 10 months, about 3 months to about 11 months, about 3 months to about 12 months, about 3 months to about 13 months, about 3 months to about 14 months, about 3 months to about 15 months, about 3 months to about 16 months, about 3 months to about 17 months, about 3 months to about 18 months, about 4 months to about 5 months, about 4 months to about 6 months, about 4 months to about 7 months, about 4 months to about 8 months, about 4 months to about 9 months, about 4 months to about 10 months, about 4 months to about 11 months, about 4 months to about 12 months, about 4 months to about 13 months, about 4 months to about 14 months, about 4 months to about 15 months, about 4 months to about 16 months, about 4 months to about 17 months, about 4 months to about 18 months, about 5 months to about 6 months, about 5 months to about 7 months, about 5 months to about 8 months, about 5 months to about 9 months, about 5 months to about 10 months, about 5 months to about 11 months, about 5 months to about 12 months, about 5 months to about 13 months, about 5 months to about 14 months, about 5 months to about 15 months, about 5 months to about 16 months, about 5 months to about 17 months, about 5 months to about 18 months, about 6 months to about 7 months, about 6 months to about 8 months, about 6 months to about 9 months, about 6 months to about 10 months, about 6 months to about 11 months, about 6 months to about 12 months, about 6 months to about 13 months, about 6 months to about 14 months, about 6 months to about 15 months, about 6 months to about 16 months, about 6 months to about 17 months, about 6 months to about 18 months, about 7 months to about 8 months, about 7 months to about 9 months, about 7 months to about 10 months, about 7 months to about 11 months, about 7 months to about 12 months, about 7 months to about 13 months, about 7 months to about 14 months, about 7 months to about 15 months, about 7 months to about 16 months, about 7 months to about 17 months, about 7 months to about 18 months, about 8 months to about 9 months, about 8 months to about 10 months, about 8 months to about 11 months, about 8 months to about 12 months, about 8 months to about 13 months, about 8 months to about 14 months, about 8 months to about 15 months, about 8 months to about 16 months, about 8 months to about 17 months, about 8 months to about 18 months, about 9 months to about 10 months, about 9 months to about 11 months, about 9 months to about 12 months, about 9 months to about 13 months, about 9 months to about 14 months, about 9 months to about 15 months, about 9 months to about 16 months, about 9 months to about 17 months, about 9 months to about 18 months, about 10 months to about 11 months, about 10 months to about 12 months, about 10 months to about 13 months, about 10 months to about 14 months, about 10 months to about 15 months, about 10 months to about 16 months, about 10 months to about 17 months, about 10 months to about 18 months, about 11 months to about 12 months, about 11 months to about 13 months, about 11 months to about 14 months, about 11 months to about 15 months, about 11 months to about 16 months, about 11 months to about 17 months, about 1 months to about 18 months, about 12 months to about 13 months, about 12 months to about 14 months, about 12 months to about 15 months, about 12 months to about 16 months, about 12 months to about 17 months, about 12 months to about 18 months, about 13 months to about 14 months, about 13 months to about 15 months, about 13 months to about 16 months, about 13 months to about 17 months, about 13 months to about 18 months, about 14 months to about 15 months, about 14 months to about 16 months, about 14 months to about 17 months, about 14 months to about 18 months, about 15 months to about 16 months, about 15 months to about 17 months, about 15 months to about 18 months, about 16 months to about 17 months, about 16 months to about 18 months, or about 17 months to about 18 months.
44. The grip enhancing composition according to any one of embodiments 1-41, having a stability of about 1 year, about 2 years, about 3 years, about 4 years, or about 5 years or at least 1 year, at least 2 years, at least 3 years, at least 4 years, or at least 5 years or at most 1 year, at most 2 years, at most 3 years, at most 4 years, or at most 5 years or about 1 year to about 2 years, about 1 year to about 3 years, about 1 year to about 4 years, about 1 year to about 5 years, about 2 years to about 3 years, about 2 years to about 4 years, about 2 years to about 5 years, about 3 years to about 4 years, about 3 years to about 5 years, or about 4 years to about 5 years.
45. The grip enhancing composition according to any one of embodiments 1-44, formulated as a single-phase formulation, a biphasic formulation, or a multiphasic formulation.
46. The grip enhancing composition according to any one of embodiments 1-44, formulated as a liquid composition, a colloidal composition, a semi-solid composition, or a solid composition.
47. The grip enhancing composition according to any one of embodiments 1-44, formulated as a liquid aerosol, a foam, an emulsion, a gel, a sol, or a solid sol.
48. The grip enhancing composition according to any one of embodiments 1-44, formulated as a spray, a liquid aerosol, a wash, a lotion, a cream, a foam, an emulsion, a suspension, a gel, a sol, a wet powder, a pressed powder, or a solid sol.
49. The grip enhancing composition according to any one of embodiments 1-48, wherein the grip enhancing composition does not comprise, consist essentially of, or consist of ozone.
50. The grip enhancing composition according to any one of embodiments 1-49, wherein the grip enhancing composition does not comprise, consist essentially of, or consist of hydrogen peroxide.
51. The grip enhancing composition according to any one of embodiments 1-50, wherein the grip enhancing composition does not comprise, consist essentially of, or consist of a dissolved inorganic carbon.
52. The grip enhancing composition according to any one of embodiments 1-51, wherein the grip enhancing composition does not comprise, consist essentially of, or consist of sodium dihydrogen phosphate.
53. The grip enhancing composition according to any one of embodiments 1-52, wherein the grip enhancing composition does not comprise, consist essentially of, or consist of an alcohol.
54. A kit comprising, consisting essentially of, or consisting of a composition as defined in any one of embodiments 1-53.
55. The kit according to embodiment 54, further comprising, consisting essentially of, or consisting of one or more delivery or application systems, and/or instructions, and/or a container.
56. A kit comprising a first component including a composition comprising, consisting essentially of, or consisting of one or more grip enhancing component and one more hydrophilic clays.
57. A kit comprising a first component including a composition comprising, consisting essentially of, or consisting of one or more grip enhancing component and one more polysaccharides.
58. A kit according to embodiment 56 or embodiment 57, further comprising, consisting essentially of, or consisting of a second component including a composition comprising silica.
59. A kit according to any one of embodiments 56-58, further comprising, consisting essentially of, or consisting of a third component including a composition comprising hypochlorous acid or free available chlorine.
60. A kit according to any one of embodiments 56-58, further comprising, consisting essentially of, or consisting of a third component including an ascorbic acid composition.
61. A kit according to any one of embodiments 56-58, further comprising, consisting essentially of, or consisting of a third component including a composition comprising, consisting essentially of, or consisting of hypochlorous acid or free available chlorine and a fourth component including an ascorbic acid composition.
62. The kit according to any one of Embodiments 56-61, further comprising, consisting essentially of, or consisting of one or more antimicrobial agents, one or more fragrances, one or more delivery or application systems, and/or instructions, and/or a container.
63. The kit according to any one of embodiments 56-62, wherein the one or more grip enhancers comprise, consist essentially of, or consist of one or more chalks, one or more talc, one or more resins, one or more rosins, or any combination thereof.
64. The kit according to embodiment 63, wherein the one or more chalks include magnesium carbonate, calcium carbonate, or any combination thereof.
65. The kit according to embodiment 63 or embodiment 64, wherein the one or more talcs include magnesium silicate, calcium silicate, or any combination thereof.
66. The kit according to any one of embodiments 56-65, wherein the one or more grip enhancers in an amount of 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%. 9%, 10%, 15%, 20%, 25%, 30%, 35%, 40%, 45%, 50%, 55%, 60%, 65%, 70%, 75%, 80%, 85%, 90%, or 95% by weight of the composition; at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% by weight of the composition; at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, at most 10%, at most 15%, at most 20%, at most 25%, at most 30%, at most 35%, at most 40%, 40%, at most 45%, at most 50%, at most 55%, at most 60%, at most 65%, at most 70%, at most 75%, at most 80%, at most 85%, at most 90%, or at most 95% by weight of the composition, or any combination thereof; about 0.01% to about 0.05%, about 0.01% to about 0.075%, about 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 15%, about 0.1% to about 20%, about 0.1% to about 25%, about 0.1% to about 30%, about 0.1% to about 35%, about 0.1% to about 40%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.25% to about 15%, about 0.25% to about 20%, about 0.25% to about 25%, about 0.25% to about 30%, about 0.25% to about 35%, about 0.25% to about 40%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.5% to about 15%, about 0.5% to about 20%, about 0.5% to about 25%, about 0.5% to about 30%, about 0.5% to about 35%, about 0.5% to about 40%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 0.75% to about 15%, about 0.75% to about 20%, about 0.75% to about 25%, about 0.75% to about 30%, about 0.75% to about 35%, about 0.75% to about 40%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 15%, about 1% to about 20%, about 1% to about 25%, about 1% to about 30%, about 1% to about 35%, about 1% to about 40%, about 2% to about 3%, about 2% to about 4%, about 2% to about 5%, about 2% to about 6%, about 2% to about 7%, about 2% to about 8%, about 2% to about 9%, about 2% to about 10%, about 2% to about 15%, about 2% to about 20%, about 2% to about 25%, about 2% to about 30%, about 2% to about 35%, about 2% to about 40%, about 3% to about 4%, about 3% to about 5%, about 3% to about 6%, about 3% to about 7%, about 3% to about 8%, about 3% to about 9%, about 3% to about 10%, about 3% to about 15%, about 3% to about 20%, about 3% to about 25%, about 3% to about 30%, about 3% to about 35%, about 3% to about 40%, about 4% to about 5%, about 4% to about 6%, about 4% to about 7%, about 4% to about 8%, about 4% to about 9%, about 4% to about 10%, about 4% to about 15%, about 4% to about 20%, about 4% to about 25%, about 4% to about 30%, about 4% to about 35%, about 4% to about 40%, about 5% to about 6%, about 5% to about 7%, about 5% to about 8%, about 5% to about 9%, about 5% to about 10%, about 5% to about 15%, about 5% to about 20%, about 5% to about 25%, about 5% to about 30%, about 5% to about 35%, about 5% to about 40%, about 6% to about 7%, about 6% to about 8%, about 6% to about 9%, about 6% to about 10%, about 6% to about 15%, about 6% to about 20%, about 6% to about 25%, about 6% to about 30%, about 6% to about 35%, about 6% to about 40%, about 7% to about 8%, about 7% to about 9%, about 7% to about 10%, about 7% to about 15%, about 7% to about 20%, about 7% to about 25%, about 7% to about 30%, about 7% to about 35%, about 7% to about 40%, about 8% to about 9%, about 8% to about 10%, about 8% to about 15%, about 8% to about 20%, about 8% to about 25%, about 8% to about 30%, about 8% to about 35%, about 8% to about 40%, about 9% to about 10%, about 9% to about 15%, about 9% to about 20%, about 9% to about 25%, about 9% to about 30%, about 9% to about 35%, about 9% to about 40%, about 10% to about 15%, about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, or about 35% to about 40% by weight of the composition; or about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 20% to about 30%, about 20% to about 40%, about 20% to about 50%, about 20% to about 60%, about 20% to about 70%, about 20% to about 80%, about 20% to about 90%, about 20% to about 95%, about 30% to about 40%, about 30% to about 50%, about 30% to about 60%, about 30% to about 70%, about 30% to about 80%, about 30% to about 90%, about 30% to about 95%, about 40% to about 50%, about 40% to about 60%, about 40% to about 70%, about 40% to about 80%, about 40% to about 90%, about 40% to about 95%, about 50% to about 60%, about 50% to about 70%, about 50% to about 80%, about 50% to about 90%, about 50% to about 95%, about 60% to about 70%, about 60% to about 80%, about 60% to about 90%, about 60% to about 95%, about 70% to about 80%, about 70% to about 90%, about 70% to about 95%, about 80% to about 90%, about 80% to about 95%, or about 90% to about 95% by weight of the composition.
67. The kit according to any one of embodiments 56 or 58-66, wherein the one or more hydrophilic clays has thixotrophic properties.
68. The kit according to any one of embodiments 56-67, wherein the one or more hydrophilic clays comprise, consist essentially of, or consist of one or more bentonite clays, one or more hectorite clays, one or more kaolinite clays, one or more silicate clays, or any combination thereof.
69. The kit according to embodiment 68, wherein the one or more bentonite clays include a potassium bentonite clay, a sodium bentonite clay, a calcium bentonite clay, an aluminum bentonite clay, or any combination thereof.
70. The kit according to embodiment 68, wherein the one or more silicate clays include a lithium magnesium sodium silicate clay, a sodium magnesium fluorosilicate clay, or any combination thereof.
71. The kit according to any one of embodiments 56-70, wherein the one or more hydrophilic clays in an amount of 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%. 9%, or 10% by weight of the composition; at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, or at least 10% by weight of the composition; at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, or at most 10% by weight of the composition, or any combination thereof; or about 0.01% to about 0.05%, 0.01% to about 0.075%, about 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10% or about 9% to about 10% by weight of the composition.
72. The grip kit according to any one of embodiments 57-71, wherein the one or more polysaccharides comprise, consist essentially of, or consist of cellulose, one or more cellulose ether derivatives, one or more cellulose ester derivatives, any salt thereof, or any combination thereof.
73. The kit according to embodiment 72, wherein the one or more cellulose ether derivatives include ethyl cellulose, methyl cellulose, ethylmethyl cellulose, hydroxyethyl cellulose, hydroxypropyl cellulose, hydroxyethylmethyl cellulose, hydroxypropylmethyl cellulose, ethyl hydroxyethyl cellulose, carboxymethyl cellulose, or any salt thereof.
74. The kit according to embodiment 73, wherein the one or more cellulose ester derivatives include cellulose acetate, cellulose acetate phthalate, cellulose acetate butyrate, cellulose acetate trimelitate, hydroxupropylmethyl cellulose phthalate, or any salt thereof.
75. The kit according to any one of embodiments 56-74, wherein the one or more polysaccharides is in an amount of 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%. 9%, or 10% by weight of the composition; at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, or at least 10% by weight of the composition; at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, or at most 10% by weight of the composition, or any combination thereof; or about 0.01% to about 0.05%, 0.01% to about 0.075%, about 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10% or about 9% to about 10% by weight of the composition.
76. The kit according to any one of embodiments 58-75, wherein the silica is in an amount of 0.01%, 0.025%, 0.05%, 0.075%, 0.1%, 0.2%, 0.25%, 0.3%, 0.4%, 0.5%, 0.6%, 0.7%, 0.75%, 0.8%, 0.9%, 1%, 2%, 3%, 4%, 5%, 6%, 7%, 8%. 9%, 10%, 15%, 20%, 25%, 30%, 35%, or 40% by weight of the composition or at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, least 10%, least 15%, least 20%, least 25%, least 30%, least 35%, or least 40% by weight of the composition or at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, most 10%, most 15%, most 20%, most 25%, most 30%, most 35%, or most 40% by weight of the composition, or any combination thereof; or 0.01% to about 0.05%, 0.01% to about 0.075%, 0.01% to about 0.1%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 15%, about 0.1% to about 20%, about 0.1% to about 25%, about 0.1% to about 30%, about 0.1% to about 35%, about 0.1% to about 40%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 8%, about 0.25% to about 9%, about 0.25% to about 10%, about 0.25% to about 15%, about 0.25% to about 20%, about 0.25% to about 25%, about 0.25% to about 30%, about 0.25% to about 35%, about 0.25% to about 40%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.5% to about 15%, about 0.5% to about 20%, about 0.5% to about 25%, about 0.5% to about 30%, about 0.5% to about 35%, about 0.5% to about 40%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 8%, about 0.75% to about 9%, about 0.75% to about 10%, about 0.75% to about 15%, about 0.75% to about 20%, about 0.75% to about 25%, about 0.75% to about 30%, about 0.75% to about 35%, about 0.75% to about 40%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 15%, about 1% to about 20%, about 1% to about 25%, about 1% to about 30%, about 1% to about 35%, about 1% to about 40%, about 2% to about 3%, about 2% to about 4%, about 2% to about 5%, about 2% to about 6%, about 2% to about 7%, about 2% to about 8%, about 2% to about 9%, about 2% to about 10%, about 2% to about 15%, about 2% to about 20%, about 2% to about 25%, about 2% to about 30%, about 2% to about 35%, about 2% to about 40%, about 3% to about 4%, about 3% to about 5%, about 3% to about 6%, about 3% to about 7%, about 3% to about 8%, about 3% to about 9%, about 3% to about 10%, about 3% to about 15%, about 3% to about 20%, about 3% to about 25%, about 3% to about 30%, about 3% to about 35%, about 3% to about 40%, about 4% to about 5%, about 4% to about 6%, about 4% to about 7%, about 4% to about 8%, about 4% to about 9%, about 4% to about 10%, about 4% to about 15%, about 4% to about 20%, about 4% to about 25%, about 4% to about 30%, about 4% to about 35%, about 4% to about 40%, about 5% to about 6%, about 5% to about 7%, about 5% to about 8%, about 5% to about 9%, about 5% to about 10%, about 5% to about 15%, about 5% to about 20%, about 5% to about 25%, about 5% to about 30%, about 5% to about 35%, about 5% to about 40%, about 6% to about 7%, about 6% to about 8%, about 6% to about 9%, about 6% to about 10%, about 6% to about 15%, about 6% to about 20%, about 6% to about 25%, about 6% to about 30%, about 6% to about 35%, about 6% to about 40%, about 7% to about 8%, about 7% to about 9%, about 7% to about 10%, about 7% to about 15%, about 7% to about 20%, about 7% to about 25%, about 7% to about 30%, about 7% to about 35%, about 7% to about 40%, about 8% to about 9%, about 8% to about 10%, about 8% to about 15%, about 8% to about 20%, about 8% to about 25%, about 8% to about 30%, about 8% to about 35%, about 8% to about 40%, about 9% to about 10%, about 9% to about 15%, about 9% to about 20%, about 9% to about 25%, about 9% to about 30%, about 9% to about 35%, about 9% to about 40%, about 10% to about 15%, about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, or about 35% to about 40% by weight of the composition.
77. The kit according to any one of embodiments 59 or 61-76, wherein the hypochlorous acid or free available chlorine is in an amount of 0.00005%, 0.00006%, 0.00007%, 0.000075%, 0.00008%, 0.00009%, 0.0001%, 0.0005%, 0.001%, 0.0015%, 0.002%, 0.0025%, 0.003%, 0.0035%, 0.004%, 0.0045%, 0.005%, 0.0055%, 0.006%, 0.007%, 0.0075%, 0.008%, 0.009%, 0.01%, 0.02%, 0.03%, 0.04% or 0.05% by weight of the composition or at least 0.00005%, at least 0.00006%, at least 0.00007%, at least 0.000075%, at least 0.00008%, at least 0.00009%, at least 0.0001%, at least 0.0005%, at least 0.001%, at least 0.0015%, at least 0.002%, at least 0.0025%, at least 0.003%, at least 0.0035%, at least 0.004%, at least 0.0045%, at least 0.005%, at least 0.0055%, at least 0.006%, at least 0.007%, at least 0.0075%, at least 0.008%, at least 0.009%, at least 0.01%, at least 0.02%, at least 0.03%, at least 0.04% or at least 0.05% by weight of the composition or at most 0.00005%, at most 0.00006%, at most 0.00007%, at most 0.000075%, at most 0.00008%, at most 0.00009%, at most 0.0001%, at most 0.0005%, at most 0.001%, at most 0.0015%, at most 0.002%, at most 0.0025%, at most 0.003%, at most 0.0035%, at most 0.004%, at most 0.0045%, at most 0.005%, at most 0.0055%, at most 0.006%, at most 0.007%, at most 0.0075%, at most 0.008%, at most 0.009%, at most 0.01%, at most 0.02%, at most 0.03%, at most 0.04% or at most 0.05% by weight of the composition, or any combination thereof; or about 0.00075% to about 0.002%, about 0.00075% to about 0.003%, about 0.00075% to about 0.004%, about 0.00075% to about 0.005%, about 0.00075% to about 0.006%, about 0.00075% to about 0.007%, about 0.00075% to about 0.008%, about 0.00075% to about 0.009%, about 0.00075% to about 0.01%, about 0.001% to about 0.002%, about 0.001% to about 0.003%, about 0.001% to about 0.004%, about 0.001% to about 0.005%, about 0.001% to about 0.006%, about 0.001% to about 0.007%, about 0.001% to about 0.008%, about 0.001% to about 0.009%, about 0.001% to about 0.01%, about 0.002% to about 0.003%, about 0.002% to about 0.004%, about 0.002% to about 0.005%, about 0.002% to about 0.006%, about 0.002% to about 0.007%, about 0.002% to about 0.008%, about 0.002% to about 0.009%, about 0.002% to about 0.01% by weight of the composition
78. The kit according to any one of embodiments 59 or 61-76, wherein the hypochlorous acid or free available chlorine is in an amount of 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm or 400 ppm or at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm or at least 400 ppm or at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm or at most 400 ppm, or any combination thereof; or about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm or about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.
79. The kit according to any one of embodiments 60-78, wherein the ascorbic acid is in an amount of 0.00005%, 0.00006%, 0.00007%, 0.000075%, 0.00008%, 0.00009%, 0.0001%, 0.0005%, 0.001%, 0.0015%, 0.002%, 0.0025%, 0.003%, 0.0035%, 0.004%, 0.0045%, 0.005%, 0.0055%, 0.006%, 0.007%, 0.0075%, 0.008%, 0.009%, 0.01%, 0.02%, 0.03%, 0.04% or 0.05% by weight of the composition or at least 0.00005%, at least 0.00006%, at least 0.00007%, at least 0.000075%, at least 0.00008%, at least 0.00009%, at least 0.0001%, at least 0.0005%, at least 0.001%, at least 0.0015%, at least 0.002%, at least 0.0025%, at least 0.003%, at least 0.0035%, at least 0.004%, at least 0.0045%, at least 0.005%, at least 0.0055%, at least 0.006%, at least 0.007%, at least 0.0075%, at least 0.008%, at least 0.009%, at least 0.01%, at least 0.02%, at least 0.03%, at least 0.04% or at least 0.05% by weight of the composition or at most 0.00005%, at most 0.00006%, at most 0.00007%, at most 0.000075%, at most 0.00008%, at most 0.00009%, at most 0.0001%, at most 0.0005%, at most 0.001%, at most 0.0015%, at most 0.002%, at most 0.0025%, at most 0.003%, at most 0.0035%, at most 0.004%, at most 0.0045%, at most 0.005%, at most 0.0055%, at most 0.006%, at most 0.007%, at most 0.0075%, at most 0.008%, at most 0.009%, at most 0.01%, at most 0.02%, at most 0.03%, at most 0.04% or at most 0.05% by weight of the composition, or any combination thereof; or about 0.00075% to about 0.002%, about 0.00075% to about 0.003%, about 0.00075% to about 0.004%, about 0.00075% to about 0.005%, about 0.00075% to about 0.006%, about 0.00075% to about 0.007%, about 0.00075% to about 0.008%, about 0.00075% to about 0.009%, about 0.00075% to about 0.01%, about 0.001% to about 0.002%, about 0.001% to about 0.003%, about 0.001% to about 0.004%, about 0.001% to about 0.005%, about 0.001% to about 0.006%, about 0.001% to about 0.007%, about 0.001% to about 0.008%, about 0.001% to about 0.009%, about 0.001% to about 0.01%, about 0.002% to about 0.003%, about 0.002% to about 0.004%, about 0.002% to about 0.005%, about 0.002% to about 0.006%, about 0.002% to about 0.007%, about 0.002% to about 0.008%, about 0.002% to about 0.009%, about 0.002% to about 0.01% by weight of the composition
80. The kit according to any one of embodiments 60-78, wherein the ascorbic acid is in an amount of 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm or 400 ppm or at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm or at least 400 ppm or at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm or at most 400 ppm, or any combination thereof; or about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm or about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.
81. A method to improve the grip capacity of an individual, the method comprising, consisting essentially of, or consisting of the step of applying a grip enhancing composition as defined in any one of embodiments 1-53, or applying the components as defined in any one of embodiments 54-80, to a skin region of an individual, wherein application to the skin region of the individual restores, maintains and/or improves the grip capacity of the individual, allowing the individual to more securely grip an object.
82. A method of providing protection to a skin region of an individual, the method comprising, consisting essentially of, or consisting of the step of applying a grip enhancing composition as defined in any one of embodiments 1-53, or applying the components as defined in any one of embodiments 54-80, to a skin region of an individual, wherein application to the skin region of the individual reduces, ameliorates, eliminates or prevents an irritation, a pain or other symptom that causes discomfort or displeasure to the individual, thereby providing protection to the skin region of the individual.
83. The method according to embodiment 81 or embodiment 82, wherein the skin region comprises, consists essentially of, or consists of a face, a forehead, a lip, a scalp, a neck, a shoulder, an arm, a hand, a palm of hand, a thigh, a leg, a knee, a foot, a sole of a foot, a chest, a breast, a back, a groin, a buttock, or any combination thereof.
84. A method to improve the grip capacity of an individual, the method comprising, consisting essentially of, or consisting of the step of applying a grip enhancing composition as defined in any one of embodiments 1-53, or applying the components as defined in any one of embodiments 54-80, to a first palm surface and/or first finger surface of a first hand of an individual, a second palm surface and/or second finger surface of a second hand of an individual, or a combination thereof, wherein application to the palm and finger surfaces of the individual restores, maintains and/or improves the grip capacity of the individual, allowing the individual to more securely grip an object.
85. A method to improve the grip capacity of an individual, the method comprising, consisting essentially of, or consisting of the step of applying a grip enhancing composition as defined in any one of embodiments 1-53, or applying the components as defined in any one of embodiments 54-80, to a first foot surface and/or first toe surface of a first foot of an individual, a second foot surface and/or second toe surface of a second foot of an individual, or a combination thereof, wherein application to the foot and toe surfaces of the individual reduces, ameliorates, eliminates or prevents an irritation, a pain or other symptom that causes discomfort or displeasure, thereby providing protection to the foot and toe surfaces.
86. A grip enhancing composition as defined in any one of embodiments 1-53, or components as defined in any one of embodiments 54-80, for use to improve the grip capacity of an individual.
87. A grip enhancing composition as defined in any one of embodiments 1-53, or components as defined in any one of embodiments 54-80, for use to protect a skin region of an individual.
88. Use of a grip enhancing composition as defined in any one of embodiments 1-53, or the components as defined in any one of embodiments 54-80, to improve the grip capacity of an individual.
89. Use of a grip enhancing composition as defined in any one of embodiments 1-53, or the components as defined in any one of embodiments 54-80, to protect a skin region of an individual.
90. Use of a grip enhancing composition as defined in any one of embodiments 1-53, or the components as defined in any one of embodiments 54-80, in the manufacture of a medicament to improve the grip capacity of an individual.
91. Use of a grip enhancing composition as defined in any one of embodiments 1-53, or the components as defined in any one of embodiments 54-80, in the manufacture of a medicament to protect a skin region of an individual.
92. A method to improve the grip capacity of an individual, the method comprising, consisting essentially of, or consisting of the step of applying a grip enhancing composition as defined in any one of embodiments 1-53, or applying the components as defined in any one of embodiments 54-80, to a surface of an object.
93. A grip enhancing composition as defined in any one of embodiments 1-53, or the components as defined in any one of embodiments 54-80, for use to improve the grip capacity of a surface of an object.
94. Use of a grip enhancing composition as defined in any one of embodiments 1-53, or the components as defined in any one of embodiments 54-80, to improve the grip capacity of a surface of an object.
95. Use of a grip enhancing composition as defined in any one of embodiments 1-53, or the components as defined in any one of embodiments 54-80, in the manufacture of a medicament to improve the grip capacity of a surface of an object.
96. The method of embodiments 81, 84, or 92 or the use according to any one of embodiments 93-95, wherein the object includes a piece of equipment, apparatus, implement, or other object comprising, consisting essentially of, or consisting of a gripping portion that an individual desire to grip securely to competently, proficiently and/or safely execute an activity being performed using the object.
97. The method of embodiments 81, 84 or 92 or the use according to any one of embodiments 93-95, wherein the surface is a griping or contact portion of the object.
98. The method of embodiments 81, 84, or 92, 96 or 97 or the use according to any one of embodiments 93-97, wherein the object includes a bar, a barbell, a dumbbell, a kettlebell, an uneven bar, a ring, a balance beam, a pommel horse, a vault, a mat, a floor, a pole, a wrist strap, an inside glove surface, an outside glove surface, a baseball bat, a softball bat, a cricket bat, a golf club, an ice hockey stick, a field hockey stick, a tennis racket, a racquetball racket, a shoe, a javelin, a spear, a gun, a tool, a rope, a disc, a frisbee, a saddle, an oar, a piece of equipment, a cable, a rock and a tree branch.
99. The method according to any one of embodiments 81-85, 92, or 96-98 or the use according to any one of embodiments 86-91 or 93-98, wherein the grip enhancing composition is applied by rubbing, pouring, sprinkling, or spraying.
100. The method according to any one of embodiments 81-85, 92, or 96-99 or the use according to any one of embodiments 86-91 or 93-99, wherein the grip enhancing composition is applied using a solid support.
101. The method according to embodiment 100, wherein the solid support is a wipe, a patch, a strip, a towelette, a towel, an inside and/or outside surface of a mitt, an inside and/or outside surface of a glove or a mask.
102. The method according to any one of embodiments 81-85, 92, or 96-99 or the use according to any one of embodiments 86-91 or 93-99, wherein the grip enhancing composition is applied using a delivery device.
103. The method according to embodiment 102, wherein the delivery device is an aerosol dispenser, a pump spray, a trigger spray or a squeeze bottle.
104. The method according to any one of embodiments 81-85, 92, or 96-103 or the use according to any one of embodiments 86-91 or 93-103, wherein the grip enhancing composition is applied multiple times per day.
105. The method according to embodiment 104, wherein the multiple times per day is twice a day, three times a day, four time a day, five times a day, six times a day or as often as desired.
106. The method according to any one of embodiments 81-85, 92, or 96-105 or the use according to any one of embodiments 86-91 or 93-105, wherein the grip capacity of the individual as measured by grip pressure is improved by about 10 mmHg, about 20 mmHg, about 30 mmHg, about 40 mmHg, about 50 mmHg, about 60 mmHg, about 70 mmHg, about 80 mmHg, about 90 mmHg, or about 100 mmHg; or by at least 10 mmHg, at least 20 mmHg, at least 30 mmHg, at least 40 mmHg, at least 50 mmHg, at least 60 mmHg, at least 70 mmHg, at least 80 mmHg, at least 90 mmHg, or by at least 100 mmHg; or at most 10 mmHg, at most 20 mmHg, at most 30 mmHg, at most 40 mmHg, at most 50 mmHg, at most 60 mmHg, at most 70 mmHg, at most 80 mmHg, at most 90 mmHg, or at most 100 mmHg, or any combination thereof; or by about 10 mmHg to about 20 mmHg, about 10 mmHg to about 30 mmHg, about 10 mmHg to about 40 mmHg, about 10 mmHg to about 50 mmHg, about 10 mmHg to about 60 mmHg, about 10 mmHg to about 70 mmHg, about 10 mmHg to about 80 mmHg, about 10 mmHg to about 90 mmHg, about 10 mmHg to about 100 mmHg, about 20 mmHg to about 30 mmHg, about 20 mmHg to about 40 mmHg, about 20 mmHg to about 50 mmHg, about 20 mmHg to about 60 mmHg, about 20 mmHg to about 70 mmHg, about 20 mmHg to about 80 mmHg, about 20 mmHg to about 90 mmHg, about 20 mmHg to about 100 mmHg, about 30 mmHg to about 40 mmHg, about 30 mmHg to about 50 mmHg, about 30 mmHg to about 60 mmHg, about 30 mmHg to about 70 mmHg, about 30 mmHg to about 80 mmHg, about 30 mmHg to about 90 mmHg, about 30 mmHg to about 100 mmHg, about 40 mmHg to about 50 mmHg, about 40 mmHg to about 60 mmHg, about 40 mmHg to about 70 mmHg, about 40 mmHg to about 80 mmHg, about 40 mmHg to about 90 mmHg, about 40 mmHg to about 100 mmHg, about 50 mmHg to about 60 mmHg, about 50 mmHg to about 70 mmHg, about 50 mmHg to about 80 mmHg, about 50 mmHg to about 90 mmHg, about 50 mmHg to about 100 mmHg, about 60 mmHg to about 70 mmHg, about 60 mmHg to about 80 mmHg, about 60 mmHg to about 90 mmHg, about 60 mmHg to about 100 mmHg, about 70 mmHg to about 80 mmHg, about 70 mmHg to about 90 mmHg, about 70 mmHg to about 100 mmHg, about 80 mmHg to about 90 mmHg, about 80 mmHg to about 100 mmHg, or about 90 mmHg to about 100 mmHg.
107. The method according to any one of embodiments 81-85, 92, or 96-106 or the use according to any one of embodiments 86-91 or 93-106, wherein the grip capacity of the individual as measured by grip force is improved by about 5 psi, about 10 psi, about 15 psi, about 20 psi, about 25 psi, about 30 psi, about 35 psi, about 40 psi, about 45 psi, or about 50 psi; or by at least 5 psi, at least 10 psi, at least 15 psi, at least 20 psi, at least 25 psi, at least 30 psi, at least 35 psi, at least 40 psi, at least 45 psi, or at least 50 psi; or by at most 5 psi, at most 10 psi, at most 15 psi, at most 20 psi, at most 25 psi, at most 30 psi, at most 35 psi, at most 40 psi, at most 45 psi, or at most 50 psi, or any combination thereof; or by about 5 psi to about 10 psi, about 5 psi to about 20 psi, about 5 psi to about 30 psi, about 5 psi to about 40 psi, about 5 psi to about 50 psi, about 10 psi to about 20 psi, about 10 psi to about 30 psi, about 10 psi to about 40 psi, about 10 psi to about 50 psi, about 20 psi to about 30 psi, about 20 psi to about 40 psi, about 20 psi to about 50 psi, about 30 psi to about 40 psi, about 30 psi to about 50 psi, or about 40 psi to about 50 psi.
108. The method according to any one of embodiments 81-85, 92, or 96-107 or the use according to any one of embodiments 86-91 or 93-107, wherein the grip capacity of the individual as measured by normal force slippage is improved by about 10 mN, about 20 mN, about 30 mN, about 40 mN, about 50 mN, about 60 mN, about 70 mN, about 80 mN, about 90 mN, or about 100 mN; or by at least 10 mN, at least 20 mN, at least 30 mN, at least 40 mN, at least 50 mN, at least 60 mN, at least 70 mN, at least 80 mN, at least 90 mN, or at least 100 mN; or by at most 10 mN, at most 20 mN, at most 30 mN, at most 40 mN, at most 50 mN, at most 60 mN, at most 70 mN, at most 80 mN, at most 90 mN, or at most 100 mN, or any combination thereof; or by about 10 mN to about 20 mN, about 10 mN to about 30 mN, about 10 mN to about 40 mN, about 10 mN to about 50 mN, about 10 mN to about 60 mN, about 10 mN to about 70 mN, about 10 mN to about 80 mN, about 10 mN to about 90 mN, about 10 mN to about 100 mN, about 20 mN to about 30 mN, about 20 mN to about 40 mN, about 20 mN to about 50 mN, about 20 mN to about 60 mN, about 20 mN to about 70 mN, about 20 mN to about 80 mN, about 20 mN to about 90 mN, about 20 mN to about 100 mN, about 30 mN to about 40 mN, about 30 mN to about 50 mN, about 30 mN to about 60 mN, about 30 mN to about 70 mN, about 30 mN to about 80 mN, about 30 mN to about 90 mN, about 30 mN to about 100 mN, about 40 mN to about 50 mN, about 40 mN to about 60 mN, about 40 mN to about 70 mN, about 40 mN to about 80 mN, about 40 mN to about 90 mN, about 40 mN to about 100 mN, about 50 mN to about 60 mN, about 50 mN to about 70 mN, about 50 mN to about 80 mN, about 50 mN to about 90 mN, about 50 mN to about 100 mN, about 60 mN to about 70 mN, about 60 mN to about 80 mN, about 60 mN to about 90 mN, about 60 mN to about 100 mN, about 70 mN to about 80 mN, about 70 mN to about 90 mN, about 70 mN to about 100 mN, about 80 mN to about 90 mN, about 80 mN to about 100 mN, or about 90 mN to about 100 mN.
109. The method according to any one of embodiments 81-85, 92, or 96-108 or the use according to any one of embodiments 86-91 or 93-108, wherein the grip capacity of the individual as measured by the pulling force slippage is improved by about 0.01 N, about 0.02 N, about 0.03 N, about 0.04 N, about 0.05 N, about 0.06 N, about 0.07 N, about 0.08 N, about 0.09 N, about 0.1 N, about 0.2 N, about 0.3 N, about 0.4 N, about 0.5 N, about 0.6 N, about 0.7 N, about 0.8 N, about 0.9 N, about 1 N, about 2 N, about 3 N, about 4 N, or about 5 N; or by at least 0.01 N, at least 0.02 N, at least 0.03 N, at least 0.04 N, at least 0.05 N, at least 0.06 N, at least 0.07 N, at least 0.08 N, at least 0.09 N, at least 0.1 N, at least 0.2 N, at least 0.3 N, at least 0.4 N, at least 0.5 N, at least 0.6 N, at least 0.7 N, at least 0.8 N, at least 0.9 N, at least 1 N, at least 2 N, at least 3 N, at least 4 N, or at least 5 mN; or by at most 0.01 N, at most 0.02 N, at most 0.03 N, at most 0.04 N, at most 0.05 N, at most 0.06 N, at most 0.07 N, at most 0.08 N, at most 0.09 N, at most 0.1 N, at most 0.2 N, at most 0.3 N, at most 0.4 N, at most 0.5 N, at most 0.6 N, at most 0.7 N, at most 0.8 N, at most 0.9 N, at most 1 N, at most 2 N, at most 3 N, at most 4 N, or at most 5 N, or any combination thereof; or by about 0.01 N to about 0.05 N, about 0.01 N to about 0.1 N, about 0.01 N to about 0.5 N, about 0.01 N to about 1 N, about 0.01 N to about 2 N, about 0.01 N to about 3 N, about 0.01 N to about 4 N, about 0.01 N to about 5 N, about 0.05 N to about 0.1 N, about 0.05 N to about 0.5 N, about 0.05 N to about 1 N, about 0.05 N to about 2 N, about 0.05 N to about 3 N, about 0.05 N to about 4 N, about 0.05 N to about 5 N, about 0.1 N to about 0.5 N, about 0.1 N to about 1 N, about 0.1 N to about 2 N, about 0.1 N to about 3 N, about 0.1 N to about 4 N, about 0.1 N to about 5 N, about 0.5 N to about 1 N, about 0.5 N to about 2 N, about 0.5 N to about 3 N, about 0.5 N to about 4 N, about 0.5 N to about 5 N, about 1 N to about 2 N, about 1 N to about 3 N, about 1 N to about 4 N, about 1 N to about 5 N, about 2 N to about 3 N, about 2 N to about 4 N, about 2 N to about 5 N, about 3 N to about 4 N, about 3 N to about 5 N, or about 4 N to about 5 N.
110. The method according to any one of embodiments 81-85, 92, or 96-109 or the use according to any one of embodiments 86-91 or 93-109, wherein the grip capacity of the individual as measured by grip friction speed is improved by about 1 mm/s, about 5 mm/s, about 10 mm/s, about 15 mm/s, about 20 mm/s, about 25 mm/s, about 30 mm/s, about 35 mm/s, about 40 mm/s, about 45 mm/s, or about 50 mm/s; or by at least 1 mm/s, at least 5 mm/s, at least 10 mm/s, at least 15 mm/s, at least 20 mm/s, at least 25 mm/s, at least 30 mm/s, at least 35 mm/s, at least 40 mm/s, at least 45 mm/s, or at least 50 mm/s; or by at most 1 mm/s, at most 5 mm/s, at most 10 mm/s, at most 15 mm/s, at most 20 mm/s, at most 25 mm/s, at most 30 mm/s, at most 35 mm/s, at most 40 mm/s, at most 45 mm/s, or at most 50 mm/s, or any combination thereof; or by about 1 mm/s to about 5 mm/s, about 1 mm/s to about 10 mm/s, about 1 mm/s to about 20 mm/s, about 1 mm/s to about 30 mm/s, about 1 mm/s to about 40 mm/s, about 1 mm/s to about 50 mm/s, about 5 mm/s to about 10 mm/s, about 5 mm/s to about 20 mm/s, about 5 mm/s to about 30 mm/s, about 5 mm/s to about 40 mm/s, about 5 mm/s to about 50 mm/s, about 10 mm/s to about 20 mm/s, about 10 mm/s to about 30 mm/s, about 10 mm/s to about 40 mm/s, about 10 mm/s to about 50 mm/s, about 20 mm/s to about 30 mm/s, about 20 mm/s to about 40 mm/s, about 20 mm/s to about 50 mm/s, about 30 mm/s to about 40 mm/s, about 30 mm/s to about 50 mm/s, or about 40 mm/s to about 50 mm/s.
111. The method according to any one of embodiments 81-85, 92, or 96-110 or the use according to any one of embodiments 86-91 or 93-110, wherein the method or use reduces unwanted side effects associated with block or powdered chalk and alcohol-based liquid chalk compositions.
112. The method according to any one of embodiments 81-85, 92, or 96-111 or the use according to any one of embodiments 86-91 or 93-111, wherein the method or use reduces the amount of grip enhancer that becomes airborne as dust particles due to application or use.
113. The method according to any one of embodiments 81-85, 92, or 96-112 or the use according to any one of embodiments 86-91 or 93-112, wherein the method or use reduces eye irritation, respiratory discomfort, or both.
114. The method according to any one of embodiments 81-85, 92, or 96-113 or the use according to any one of embodiments 86-91 or 93-113, wherein the method or use reduces skin chapped skin, dry skin, cracked skin, irritated skin, itchiness, skin tightness, rough skin, red plaques, discomfort, pain, or any combination thereof.
115. The grip enhancing composition according to any one of embodiments 1-53, or applying the components as defined in any one of embodiments 54-80, the method according to any one of embodiments 81-85, 92, or 96-114 or the use according to any one of embodiments 86-91 or 93-114, wherein the grip enhancing composition has reduced flammability or is not flammable.
116. The grip enhancing composition according to any one of embodiments 1-53, or the components as defined in any one of embodiments 54-80, the method according to any one of embodiments 81-85, 92, or 96-115 or the use according to any one of embodiments 86-91 or 93-115, wherein the grip enhancing composition reduces unwanted deposition of the one or more grip enhancers on fabric, hair, or skin on areas that the grip enhancing composition is not directly applied.

### EXAMPLES

The following non-limiting examples are provided for illustrative purposes only to facilitate a more complete understanding of representative embodiments now contemplated. These examples should not be construed to limit any of the embodiments described in the present specification, including those pertaining to the compositions, methods and uses disclosed herein.

### Example 1

### Grip Enhancing Compositions

This example illustrates how to formulate a grip enhancing composition. The components listed below were mixed at ambient temperature to ensure a uniform mixture. The pH of the mixture was adjusted to 7.5. The viscosity is about 500 Pa to about 1,500 mPa.

Formulations for Grip Enhancing Composition

| Components | F1 | F2 | F3 | F4 | F5 | F6 | F7 | F8 |
|---|---|---|---|---|---|---|---|---|
| Chalk¹ | 15% | 18% | 20% | 23% | 25% | 28% | 32% | 35% |
| Silicate clay² | 1.8% | 1.8% | 1.8% | 1.8% | 1.8% | 1.8% | 1.8% | 1.8% |
| Water | q.s. | q.s. | q.s. | q.s. | q.s. | q.s. | q.s. | q.s. |

¹Magnesium carbonate.
²LAPONITE ® XL21.

This example illustrates how to formulate a grip enhancing composition. The components listed below were mixed at ambient temperature to ensure a uniform mixture. The pH of the mixture was adjusted to 7.5. The viscosity is about 500 Pa to about 1,500 mPa.

Formulations for Grip Enhancing Composition

| Components | F9 | F10 | F11 | F12 | F13 | F14 | F15 | F16 |
|---|---|---|---|---|---|---|---|---|
| Chalk¹ | 15% | 18% | 20% | 23% | 25% | 28% | 32% | 35% |
| Silicate clay² | 1.8% | 1.8% | 1.8% | 1.8% | 1.8% | 1.8% | 1.8% | 1.8% |
| Phosphate | 0.04% | 0.04% | 0.04% | 0.04% | 0.04% | 0.04% | 0.04% | 0.04% |
| Water | q.s. | q.s. | q.s. | q.s. | q.s. | q.s. | q.s. | q.s. |

¹Magnesium carbonate.
²LAPONITE ® XL21.

This example illustrates how to formulate a grip enhancing composition. The components listed below were mixed at ambient temperature to ensure a uniform mixture. The pH of the mixture was adjusted to 7.5. The viscosity is about 500 Pa to about 1,500 mPa.

Formulations for Grip Enhancing Composition

| Components | F17 | F18 | F19 | F20 | F21 | F22 | F23 | F24 |
|---|---|---|---|---|---|---|---|---|
| Chalk¹ | 15% | 18% | 20% | 23% | 25% | 28% | 32% | 35% |
| Silicate clay² | 1.8% | 1.8% | 1.8% | 1.8% | 1.8% | 1.8% | 1.8% | 1.8% |
| FAC³ | 30 ppm | 30 ppm | 30 ppm | 30 ppm | 30 ppm | 30 ppm | 30 ppm | 30 ppm |
| Phosphate | 0.04% | 0.04% | 0.04% | 0.04% | 0.04% | 0.04% | 0.04% | 0.04% |
| Water | q.s. | q.s. | q.s. | q.s. | q.s. | q.s. | q.s. | q.s. |

¹Magnesium carbonate.
²LAPONITE ® XL21.
³Free available chlorine.

This example illustrates how to formulate a grip enhancing composition. The components listed below were mixed at ambient temperature to ensure a uniform mixture. The viscosity is about 500 Pa to about 1,500 mPa.

Formulations for Grip Enhancing Composition

| Components | F25 | F26 | F27 | F28 | F29 | F30 | F41 | F32 |
|---|---|---|---|---|---|---|---|---|
| Chalk¹ | 15% | 18% | 20% | 23% | 25% | 28% | 32% | 35% |
| Silicate clay² | 1.8% | 1.8% | 1.8% | 1.8% | 1.8% | 1.8% | 1.8% | 1.8% |
| Hypochlorous | 1000 | 1000 | 1000 | 1000 | 1000 | 1000 | 1000 | 1000 |
| acid | ppm | ppm | ppm | ppm | ppm | ppm | ppm | ppm |
| Phosphate | 0.04% | 0.04% | 0.04% | 0.04% | 0.04% | 0.04% | 0.04% | 0.04% |
| Water | q.s. | q.s. | q.s. | q.s. | q.s. | q.s. | q.s. | q.s. |

¹Magnesium carbonate.
²LAPONITE ® XL21.

This example illustrates how to formulate a grip enhancing composition. The components listed below were mixed at ambient temperature to ensure a uniform mixture. The viscosity is about 500 Pa to about 1,500 mPa.

Formulations for Grip Enhancing Composition

| Components | F33 | F34 | F35 | F36 | F37 | F38 | F39 | F40 |
|---|---|---|---|---|---|---|---|---|
| Chalk¹ | 15% | 18% | 20% | 23% | 25% | 28% | 32% | 35% |
| Silicate clay² | 1.8% | 1.8% | 1.8% | 1.8% | 1.8% | 1.8% | 1.8% | 1.8% |
| Hypochlorous | 1500 | 1500 | 1500 | 1500 | 1500 | 1500 | 1500 | 1500 |
| acid | ppm | ppm | ppm | ppm | ppm | ppm | ppm | ppm |
| Phosphate | 0.04% | 0.04% | 0.04% | 0.04% | 0.04% | 0.04% | 0.04% | 0.04% |
| Water | q.s. | q.s. | q.s. | q.s. | q.s. | q.s. | q.s. | q.s. |

¹Magnesium carbonate.
²LAPONITE ® XL21.

This example illustrates how to formulate a grip enhancing composition. The components listed below were mixed at ambient temperature to ensure a uniform mixture. The viscosity is about 500 Pa to about 1,500 mPa.

Formulations for Grip Enhancing Composition

| Components | F41 | F42 | F43 | F44 | F45 | F46 | F47 | F48 |
|---|---|---|---|---|---|---|---|---|
| Chalk¹ | 15% | 18% | 20% | 23% | 25% | 28% | 32% | 35% |
| Silicate clay² | 1.8% | 1.8% | 1.8% | 1.8% | 1.8% | 1.8% | 1.8% | 1.8% |
| Hypochlorous | 2000 | 2000 | 2000 | 2000 | 2000 | 2000 | 2000 | 2000 |
| acid | ppm | ppm | ppm | ppm | ppm | ppm | ppm | ppm |
| Phosphate | 0.04% | 0.04% | 0.04% | 0.04% | 0.04% | 0.04% | 0.04% | 0.04% |
| Water | q.s. | q.s. | q.s. | q.s. | q.s. | q.s. | q.s. | q.s. |

¹Magnesium carbonate.
²LAPONITE ® XL21.

This example illustrates how to formulate a grip enhancing composition. The components listed below were mixed at ambient temperature to ensure a uniform mixture. The viscosity is about 500 Pa to about 1,500 mPa.

Formulations for Grip Enhancing Composition

| Components | F49 | F50 | F51 | F52 | F53 | F54 | F55 | F56 |
|---|---|---|---|---|---|---|---|---|
| Chalk¹ | 15% | 18% | 20% | 23% | 25% | 28% | 32% | 35% |
| Silicate clay² | 1.0% | 1.0% | 1.0% | 1.0% | 1.0% | 1.0% | 1.0% | 1.0% |
| Cellulose ether | 2.0% | 2.0% | 2.0% | 2.0% | 2.0% | 2.0% | 2.0% | 2.0% |
| derivitive³ | | | | | | | | |
| Hypochlorous | 1000 | 1000 | 1000 | 1000 | 1000 | 1000 | 1000 | 1000 |
| acid | ppm | ppm | ppm | ppm | ppm | ppm | ppm | ppm |
| Phosphate | 0.04% | 0.04% | 0.04% | 0.04% | 0.04% | 0.04% | 0.04% | 0.04% |
| Water | q.s. | q.s. | q.s. | q.s. | q.s. | q.s. | q.s. | q.s. |

¹Magnesium carbonate.
²LAPONITE ® XL21.
³Hydroxyethyl cellulose.

This example illustrates how to formulate a grip enhancing composition. The components listed below were mixed at ambient temperature to ensure a uniform mixture. The viscosity is about 500 Pa to about 1,500 mPa.

Formulations for Grip Enhancing Composition

| Components | F57 | F58 | F59 | F60 | F61 | F62 | F63 | F64 |
|---|---|---|---|---|---|---|---|---|
| Chalk¹ | 15% | 18% | 20% | 23% | 25% | 28% | 32% | 35% |
| Silicate clay² | 1.0% | 1.0% | 1.0% | 1.0% | 1.0% | 1.0% | 1.0% | 1.0% |
| Cellulose ether | 2.0% | 2.0% | 2.0% | 2.0% | 2.0% | 2.0% | 2.0% | 2.0% |
| derivitive³ | | | | | | | | |
| Hypochlorous | 1500 | 1500 | 1500 | 1500 | 1500 | 1500 | 1500 | 1500 |
| acid | ppm | ppm | ppm | ppm | ppm | ppm | ppm | ppm |
| Phosphate | 0.04% | 0.04% | 0.04% | 0.04% | 0.04% | 0.04% | 0.04% | 0.04% |
| Water | q.s. | q.s. | q.s. | q.s. | q.s. | q.s. | q.s. | q.s. |

¹Magnesium carbonate.
²LAPONITE ® XL21.
³Hydroxyethyl cellulose.

This example illustrates how to formulate a grip enhancing composition. The components listed below were mixed at ambient temperature to ensure a uniform mixture. The viscosity is about 500 Pa to about 1,500 mPa.

Formulations for Grip Enhancing Composition

| Components | F65 | F66 | F67 | F68 | F69 | F70 | F71 | F72 |
|---|---|---|---|---|---|---|---|---|
| Chalk¹ | 15% | 18% | 20% | 23% | 25% | 28% | 32% | 35% |
| Silicate clay² | 1.0% | 1.0% | 1.0% | 1.0% | 1.0% | 1.0% | 1.0% | 1.0% |
| Cellulose ether | 2.0% | 2.0% | 2.0% | 2.0% | 2.0% | 2.0% | 2.0% | 2.0% |
| derivitive³ | | | | | | | | |
| Hypochlorous | 2000 | 2000 | 2000 | 2000 | 2000 | 2000 | 2000 | 2000 |
| acid | ppm | ppm | ppm | ppm | ppm | ppm | ppm | ppm |
| Phosphate | 0.04% | 0.04% | 0.04% | 0.04% | 0.04% | 0.04% | 0.04% | 0.04% |
| Water | q.s. | q.s. | q.s. | q.s. | q.s. | q.s. | q.s. | q.s. |

¹Magnesium carbonate.
²LAPONITE ® XL21.
3Hydroxyethyl cellulose.

This example illustrates how to formulate a grip enhancing composition. The components listed below were mixed at ambient temperature to ensure a uniform mixture. The pH of the mixture was adjusted to 11.0 to 12.0. The viscosity is about 500 Pa to about 1,500 mPa.

Formulations for Grip Enhancing Composition

| Components | F65 | F66 | F67 | F68 | F69 | F70 | F71 | F72 |
|---|---|---|---|---|---|---|---|---|
| Chalk¹ | 15% | 18% | 20% | 23% | 25% | 28% | 32% | 35% |
| Silicate clay² | 1.0% | 1.0% | 1.0% | 1.0% | 1.0% | 1.0% | 1.0% | 1.0% |
| Cellulose ether | 2.0% | 2.0% | 2.0% | 2.0% | 2.0% | 2.0% | 2.0% | 2.0% |
| derivitive³ | | | | | | | | |
| Phosphate | 0.04% | 0.04% | 0.04% | 0.04% | 0.04% | 0.04% | 0.04% | 0.04% |
| Electrolytic | q.s. | q.s. | q.s. | q.s. | q.s. | q.s. | q.s. | q.s. |
| Alkaline | | | | | | | | |
| Water | | | | | | | | |

¹Magnesium carbonate.
²LAPONITE ® XL21.
³Hydroxyethyl cellulose.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

1. A grip enhancing composition comprising one or more grip enhancers and one or more hydrophilic clays, wherein the grip enhancing composition does not include an alcohol.

2. The grip enhancing composition according to claim 1, wherein the one or more hydrophilic clays comprise one or more bentonite clays, one or more hectorite clays, one or more kaolinite clays, one or more silicate clays, or any combination thereof.

3. The grip enhancing composition according to claim 2, wherein the one or more bentonite clays include a potassium bentonite clay, a sodium bentonite clay, a calcium bentonite clay, an aluminum bentonite clay, or any combination thereof.

4. The grip enhancing composition according to claim 2, wherein the one or more silicate clays include a lithium magnesium sodium silicate clay, a sodium magnesium fluorosilicate clay, or any combination thereof.

5. The grip enhancing composition according to any one of claims 1-5, further comprising one or more polysaccharides.

6. The grip enhancing composition according to claim 5, wherein the one or more polysaccharides comprise cellulose, one or more cellulose ether derivatives, one or more cellulose ester derivatives, any salt thereof, or any combination thereof.

7. The grip enhancing composition according to claim 6, wherein the one or more cellulose ether derivatives include ethyl cellulose, methyl cellulose, ethylmethyl cellulose, hydroxyethyl cellulose, hydroxypropyl cellulose, hydroxyethylmethyl cellulose, hydroxypropylmethyl cellulose, ethyl hydroxyethyl cellulose, carboxymethyl cellulose, or any salt thereof.

8. The grip enhancing composition according to claim 1, wherein the one or more grip enhancers comprise one or more chalks, one or more talc, one or more resins, one or more rosins, or any combination thereof.

9. The grip enhancing composition according to claim 8, wherein the one or more chalks include magnesium carbonate, calcium carbonate, or any combination thereof.

10. The grip enhancing composition according to claim 1, further comprising a silica.

11. The grip enhancing composition according to claim 1, further comprising hypochlorous acid or free available chlorine.

12. The grip enhancing composition according to claim 1, further comprising one or more antimicrobial agents.

13. The grip enhancing composition according to claim 1, further comprising one or more fragrances.

14. The grip enhancing composition according to claim 1, further comprising one or more carriers.

15. The grip enhancing composition according to claim 1, further comprising one or more additional ingredients.

16. The grip enhancing composition according to claim 15, wherein the one or more additional ingredients include a humectant, a preservative, an emulsifier, a chelating agent, an astringent, a shine control agent, a colorant agent, a soothing agent, a calming agent, a supplement, an herbal agent, a naturopathic agent, or any combination thereof.

17. The grip enhancing composition according to claim 1, further comprising electrolytic alkaline water.

18. A kit comprising a composition as defined in claim 1.

19. The kit according to claim 18, further comprising one or more delivery or application systems, and/or instructions, and/or a container.

20. A method to improve the grip capacity of an individual, the method comprising the step of applying a grip enhancing composition as defined in claim 1 to a skin region of an individual, wherein application to the skin region of the individual restores, maintains and/or improves the grip capacity of the individual, allowing the individual to more securely grip an object.
